(19) United States
(12) Patent Application Publication
(10) Pub. No.: US 2011/0086889 A1
(43) Pub. Date: Apr. 14, 2011

(54) TETRAZOLE COMPOUNDS AS OREXIN RECEPTOR ANTAGONISTS

(76) Inventors: Hamed Aissaoui, Pulversheim (FR); Christoph Boss, Allschwil (CH); Christine Brotschi, Allschwil (CH); John Gatfield, Basel (CH); Ralf Koberstein, Lorrach (DE); Romain Siegrist, Allschwil (CH); Thierry Sifferlen, Wentzwiller (FR); Jodi Williams, Basel (CH)

(21) Appl. No.: 12/996,840

(22) PCT Filed: Jun. 10, 2009
(86) PCT No.: PCT/IB2009/052459

§ 371 Date: Dec. 8, 2010

(51) Int. Cl. A61K 31/4439; C07D 403/12; A61K 31/41; C07D 401/14; A61P 3/00; A61P 25/00

(52) U.S. Cl. 514/341; 548/253; 514/381; 546/275.4

(57) ABSTRACT

The invention relates to tetrazole compounds of formula (I) wherein X, Y, Z, R¹, R² and R³ are as described in the description; to pharmaceutically acceptable salts thereof, and to the use of such compounds use as medicaments, especially as orexin receptor antagonists.

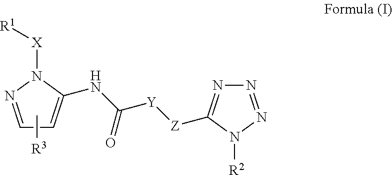

The present invention relates to tetrazole compounds of formula (I) and their use as pharmaceuticals. The invention also concerns related aspects including processes for the preparation of the compounds, pharmaceutical compositions containing one or more compounds of formula (I), and especially their use as orexin receptor antagonists.

Orexins (orexin A or OX-A and orexin B or OX-B) are novel neuropeptides found in 1998 by two research groups, orexin A is a 33 amino acid peptide and orexin B is a 28 amino acid peptide (Sakurai T. et al., Cell, 1998, 92, 573-585). Orexins are produced in discrete neurons of the lateral hypothalamus and bind to the G-protein-coupled receptors (OX₁ and OX₂ receptors). The orexin-1 receptor (OX₁) is selective for OX-A, and the orexin-2 receptor (OX₂) is capable to bind OX-A as well as OX-B. Orexins are found to stimulate food consumption in rats suggesting a physiological role for these peptides as mediators in the central feedback mechanism that regulates feeding behaviour (Sakurai T. et al., Cell, 1998, 92, 573-585). On the other hand, it was also observed that orexins regulate states of sleep and wakefulness opening potentially novel therapeutic approaches to narcolepsy as well as insomnia and other sleep disorders (Chemelli R. M. et al., Cell, 1999, 98, 437-451). Furthermore, in vitro and in vivo evidence for a critical role of orexin signaling in the ventral tegmental area in neural plasticity relevant to addiction has been published (S. L. Borgland et al. Neuron, 2006, 49, 589-601).

Thus, orexin receptors may have numerous implications in pathologies as known from the literature, such as dysthymic, mood, psychotic and anxiety disorders; diabetes and appetite, taste, eating, or drinking disorders; hypothalamic diseases; disturbed biological and circadian rhythms; sleep disturbances associated with diseases such as neurological disorders, neuropathic pain and restless leg syndrome; insomnias related to psychiatric disorders; sleep apnea; narcolepsy; idiopathic insomnias; parasomnias; benign prostatic hypertrophy; all dementias and cognitive dysfunctions in the healthy population and in psychiatric and neurologic disorders; and other diseases related to general orexin system dysfunctions. The compound (2R)-2-{(1S)-6,7-dimethoxy-1-[2-(4-trifluoromethyl-phenyl)-ethyl]-3,4-dihydro-1H-isoquinolin-2-yl}-N-methyl-2-phenyl-acetamide (WO2005/118548) is currently in clinical development for primary insomnia. In the rat, the compound has been shown for example to decrease alertness, characterized by decreases in both active wake and locomotion; and to dose-dependently increase the time spent in both REM and NREM sleep (F. Jenck et al., Nature Medicine 2007, 13, 150-155). The compound has also been shown to enhance memory function in a rat model (WO2007/105177) and is also active in a rat model of post-traumatic stress disorder (WO2009/047723).

The present invention provides tetrazole derivatives, which are non-peptide antagonists of human orexin receptors and, thus, of potential use in the treatment of diseases related to the orexin system, especially comprising all types of sleep disorders, of stress-related syndromes, of addictions (especially psychoactive substance use, abuse, seeking and reinstatement), of cognitive dysfunctions in the healthy population and in psychiatric and neurologic disorders, of eating or drinking disorders. In particular these compounds are of potential use in the treatment of eating disorders, drinking disorders, sleep disorders, or cognitive dysfunctions in psychiatric and neurologic disorders. Some tetrazole compounds are known from the CAS Registry database, however, neither their preparation nor the use of these compounds as medicaments, especially not their use as orexin receptor antagonists, is described.

1) The present invention relates to tetrazole compounds of formula (I)

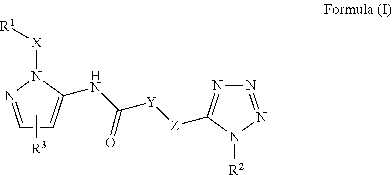

wherein
X represents —CH₂—, —CH₂—CH₂—, —CH₂—CH₂—CH₂—, or a bond;
Y represents —CH₂— which is optionally mono-substituted with (C₁₋₄)alkyl;
Z represents —CH₂—, or —S—;
R¹ represents aryl or heteroaryl, wherein said aryl or heteroaryl independently is unsubstituted, or mono-, di-, or tri-substituted, wherein the substituents are independently selected from the group consisting of (C₁₋₄)alkyl; (C₁₋₄)alkoxy; fluoroalkyl; fluoroalkoxy; halogen; N(CH₃)₂; phenyl and phenyloxy, wherein said phenyl or phenyloxy independently is unsubstituted or mono-, or di-substituted wherein the substituents are independently selected from the group consisting of (C₁₋₄)alkyl, (C₁₋₄)alkoxy, fluoroalkyl, fluoroalkoxy, and halogen; or,
in case X represents —CH₂—, R¹ additionally represents (C₁₋₆)alkyl, or (C₃₋₆)cycloalkyl;
R² represents phenyl which is unsubstituted; or mono-, di-, or tri-substituted, wherein the substituents are independently selected from the group consisting of (C₁₋₄)alkyl, (C₁₋₄)alkoxy, halogen, fluoroalkyl, and fluoroalkoxy; or
R² represents a naphthyl (especially 1-naphthyl) group or a biphenyl (especially 2-biphenyl) group which groups independently are unsubstituted, or mono-, or di-substituted, wherein the substituents are independently selected from the group consisting of (C₁₋₄)alkyl, (C₁₋₄)alkoxy, fluoroalkyl, fluoroalkoxy, and halogen; and
R³ represents hydrogen or methyl;
with the exception of the following compounds:
N-(2-Benzyl-2H-pyrazol-3-yl)-2-[1-(2,5-dimethyl-phenyl)-1H-tetrazol-5-ylsulfanyl]-acetamide (CAS Registry No. 877976-75-5);
2-[1-(2,5-Dimethyl-phenyl)-1H-tetrazol-5-ylsulfanyl]-N-(2-thiophen-2-ylmethyl-2H-pyrazol-3-yl)-acetamide (CAS Registry No. 956726-62-8);
N-[2-(3-Chloro-benzyl)-2H-pyrazol-3-yl]-2-(1-phenyl-1H-tetrazol-5-ylsulfanyl)-acetamide (CAS Registry No. 1134681-37-0);
N-[2-(2,5-Dimethyl-phenyl)-5-methyl-2H-pyrazol-3-yl]-2-(1-phenyl-1H-tetrazol-5-ylsulfanyl)-acetamide (CAS Registry No. 1134904-17-8);
N-(5-Methyl-2-phenyl-2H-pyrazol-3-yl)-2-(1-m-tolyl-1H-tetrazol-5-ylsulfanyl)-acetamide (CAS Registry No. 1134719-49-5);
N-[2-(4-Fluoro-phenyl)-5-methyl-2H-pyrazol-3-yl]-2-(1-phenyl-1H-tetrazol-5-ylsulfanyl)-acetamide (CAS Registry No. 1134706-86-7);
2-[1-(2,5-Dimethyl-phenyl)-1H-tetrazol-5-ylsulfanyl]-N-(5-methyl-2-phenyl-2H-pyrazol-3-yl)-acetamide (CAS Registry No. 1134701-04-4);
2-[1-(3,5-Dimethyl-phenyl)-1H-tetrazol-5-ylsulfanyl]-N-(5-methyl-2-p-tolyl-2H-pyrazol-3-yl)-acetamide (CAS Registry No. 1019078-82-0);
2-[1-(2,4-Dimethyl-phenyl)-1H-tetrazol-5-ylsulfanyl]-N-(2,5-dimethyl-2H-pyrazol-3-yl)-acetamide (CAS Registry No. 1015529-55-1);
N-(5-Methyl-2-p-tolyl-2H-pyrazol-3-yl)-2-[1-(4-trifluoromethoxy-phenyl)-1H-tetrazol-5-ylsulfanyl]-acetamide (CAS Registry No. 1007700-82-4);
2-[1-(2,5-Dimethyl-phenyl)-1H-tetrazol-5-ylsulfanyl]-N-(5-methyl-2-o-tolyl-2H-pyrazol-3-yl)-acetamide (CAS Registry No. 1007661-81-5);
2-[1-(2,6-Dimethyl-phenyl)-1H-tetrazol-5-ylsulfanyl]-N-(5-methyl-2-phenyl-2H-pyrazol-3-yl)-acetamide (CAS Registry No. 1002313-99-6);
2-[1-(3-Fluoro-phenyl)-1H-tetrazol-5-ylsulfanyl]-N-(5-methyl-2-phenyl-2H-pyrazol-3-yl)-acetamide (CAS Registry No. 1001835-91-1);
2-[1-(3,4-Dimethyl-phenyl)-1H-tetrazol-5-ylsulfanyl]-N-(5-methyl-2-phenyl-2H-pyrazol-3-yl)-acetamide (CAS Registry No. 1001577-66-7);
N-(5-Methyl-2-phenyl-2H-pyrazol-3-yl)-2-(1-o-tolyl-1H-tetrazol-5-ylsulfanyl)-acetamide (CAS Registry No. 957028-00-1);
N-(5-Methyl-2-phenyl-2H-pyrazol-3-yl)-2-(1-p-tolyl-1H-tetrazol-5-ylsulfanyl)-acetamide (CAS Registry No. 957027-98-4);
2-[1-(2,3-Dimethyl-phenyl)-1H-tetrazol-5-ylsulfanyl]-N-(5-methyl-2-phenyl-2H-pyrazol-3-yl)-acetamide (CAS Registry No. 956800-47-8);
2-[1-(2,4-Dimethyl-phenyl)-1H-tetrazol-5-ylsulfanyl]-N-(5-methyl-2-phenyl-2H-pyrazol-3-yl)-acetamide (CAS Registry No. 956339-59-6); and
N-(5-Methyl-2-phenyl-2H-pyrazol-3-yl)-2-(1-phenyl-1H-tetrazol-5-ylsulfanyl)-acetamide (CAS Registry No. 956203-33-1).

For avoidance of any doubt, the substituent R³ represents one substituent as defined above which is attached either to position 4 or to position 5 of the 2H-pyrazol-3-yl moiety:

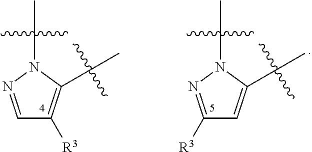

2) The invention further relates to tetrazole compounds, or pharmaceutically acceptable salts thereof, for use as medicaments, especially for use as medicaments which are active as orexin receptor antagonists; wherein said compounds are compounds of formula (I) according to embodiment 1), including the 19 above-listed specifically excluded compounds.

3) The invention further relates to novel tetrazole compounds of formula (I), which are also compounds of formula (I_{C})

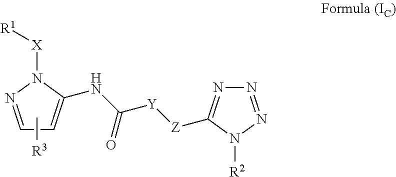

wherein
X represents —CH₂—, —CH₂—CH₂—, —CH₂—CH₂—CH₂—, or a bond;
Y represents —CH₂— which is optionally mono-substituted with (C₁₋₄)alkyl;
Z represents —CH₂—, or —S—;
R¹ represents aryl or heteroaryl, wherein said aryl or heteroaryl independently is unsubstituted, or mono-, di-, or tri-substituted, wherein the substituents are independently selected from the group consisting of (C₁₋₄)alkyl; (C₁₋₄)alkoxy; fluoroalkyl; fluoroalkoxy; halogen; N(CH₃)₂; phenyl and phenyloxy, wherein said phenyl or phenyloxy independently is unsubstituted or mono-, or di-substituted wherein the substituents are independently selected from the group consisting of (C₁₋₄)alkyl, (C₁₋₄)alkoxy, fluoroalkyl, fluoroalkoxy, and halogen; or,
in case X represents —CH₂—, R¹ additionally represents (C₁₋₆)alkyl, or (C₃₋₆)cycloalkyl;
R² represents phenyl which is unsubstituted; or mono-, di-, or tri-substituted, wherein the substituents are independently selected from the group consisting of (C₁₋₄)alkyl, (C₁₋₄)alkoxy, halogen, fluoroalkyl, and fluoroalkoxy; or
R² represents a naphthyl (especially 1-naphthyl) group or a biphenyl (especially 2-biphenyl) group which groups independently are unsubstituted, or mono-, or di-substituted, wherein the substituents are independently selected from the group consisting of (C₁₋₄)alkyl, (C₁₋₄)alkoxy, fluoroalkyl, fluoroalkoxy, and halogen; and
R³ represents hydrogen or methyl; wherein, in the particular case wherein X represents a bond, R³ is attached to position 4 of the 2H-pyrazol-3-yl moiety;
with the exception of the following compounds:
N-(2-Benzyl-2H-pyrazol-3-yl)-2-[1-(2,5-dimethyl-phenyl)-1H-tetrazol-5-ylsulfanyl]-acetamide (CAS Registry No. 877976-75-5);
2-[1-(2,5-Dimethyl-phenyl)-1H-tetrazol-5-ylsulfanyl]-N-(2-thiophen-2-ylmethyl-2H-pyrazol-3-yl)-acetamide (CAS Registry No. 956726-62-8); and
N-[2-(3-Chloro-benzyl)-2H-pyrazol-3-yl]-2-(1-phenyl-1H-tetrazol-5-ylsulfanyl)-acetamide (CAS Registry No. 1134681-37-0).

4) Another embodiment relates to compounds according to any one of embodiments 1) to 3), wherein X represents —CH₂—, —CH₂—CH₂—, or —CH₂—CH₂—CH₂— (especially X represents —CH₂—).

5) Another embodiment relates to compounds according to any one of embodiments 1) to 3), wherein X represents or a bond.

6) Another embodiment relates to compounds according to any one of embodiments 1) to 5), wherein Y represents —CH₂—.

7) Another embodiment relates to compounds according to any one of embodiments 1) to 6), wherein Z represents —CH₂—.

8) Another embodiment relates to compounds according to any one of embodiments 1) to 6), wherein Z represents —S—.

9) Another embodiment relates to compounds according to any one of embodiments 1) to 8), wherein R¹ represents aryl or heteroaryl, wherein said aryl or heteroaryl independently is unsubstituted, or mono-, di-, or tri-substituted, wherein the substituents are independently selected from the group consisting of (C₁₋₄)alkyl; (C₁₋₄)alkoxy; fluoroalkyl; fluoroalkoxy; halogen; N(CH₃)₂; phenyl and phenyloxy, wherein said phenyl or phenyloxy independently is unsubstituted or mono-, or di-substituted wherein the substituents are independently selected from the group consisting of (C₁₋₄)alkyl, (C₁₋₄)alkoxy, fluoroalkyl, fluoroalkoxy, and halogen.

10) Another embodiment relates to compounds according to any one of embodiments 1) to 9), wherein R¹ represents aryl or heteroaryl, wherein said aryl or heteroaryl independently is unsubstituted, or mono-, di-, or tri-substituted, wherein the substituents are independently selected from the group consisting of (C₁₋₄)alkyl, (C₁₋₄)alkoxy, fluoroalkyl, fluoroalkoxy, halogen, and N(CH₃)₂.

11) Another embodiment relates to compounds according to any one of embodiments 1) to 10), wherein R² represents phenyl which is unsubstituted; or mono-, di-, or tri-substituted, wherein the substituents are independently selected from the group consisting of (C₁₋₄)alkyl, (C₁₋₄)alkoxy, halogen, fluoroalkyl, and fluoroalkoxy; wherein, in case one substituent is attached to position 4, one further substituent is attached to position 2 of the phenyl ring; or

R² represents a naphthyl (especially 1-naphthyl) group or a biphenyl (especially 2-biphenyl) group which groups independently are unsubstituted, or mono-, or di-substituted, wherein the substituents are independently selected from the group consisting of (C₁₋₄)alkyl, (C₁₋₄)alkoxy, fluoroalkyl, fluoroalkoxy, and halogen.

12) Another embodiment relates to compounds according to any one of embodiments 1) to 10), wherein R² represents phenyl which is unsubstituted; or phenyl which is mono-substituted, wherein the substituent is attached to position 2 or 3 of the phenyl ring, wherein the substituent is selected from the group consisting of (C₁₋₄)alkyl, (C₁₋₄)alkoxy, halogen, fluoroalkyl, and fluoroalkoxy; or R² represents phenyl which is di-, or tri-substituted, wherein the substituents are independently selected from the group consisting of (C₁₋₄)alkyl, (C₁₋₄)alkoxy, halogen, fluoroalkyl, and fluoroalkoxy, wherein one substituent is attached to position 2 of the phenyl ring and/or two substituents are attached to positions 3 and 5 of the phenyl ring; or

R² represents a naphthyl (especially 1-naphthyl) group or a biphenyl (especially 2-biphenyl) group which groups independently are unsubstituted, or mono-, or di-substituted, wherein the substituents are independently selected from the group consisting of (C₁₋₄)alkyl, (C₁₋₄)alkoxy, fluoroalkyl, fluoroalkoxy, and halogen.

13) Another embodiment relates to compounds according to any one of embodiments 1) to 12), wherein R² represents phenyl which is unsubstituted; or phenyl which is mono-substituted, wherein the substituent is attached to position 2 or 3 of the phenyl ring, wherein the substituent is selected from the group consisting of (C₁₋₄)alkyl, (C₁₋₄)alkoxy, and halogen; or R² represents phenyl which is di- or tri-substituted (notably di-substituted), wherein the substituents are independently selected from the group consisting of (C₁₋₄)alkyl, (C₁₋₄)alkoxy, halogen, fluoroalkyl, and fluoroalkoxy (notably (C₁₋₄)alkyl, (C₁₋₄)alkoxy, and halogen), wherein one substituent is attached to position 2 of the phenyl ring.

14) Another embodiment relates to compounds according to any one of embodiments 1) to 13), wherein R³ represents hydrogen.

15) In a further embodiment, the present invention also relates to tetrazole compounds of formula (I) which are also compounds of formula (I_{P})

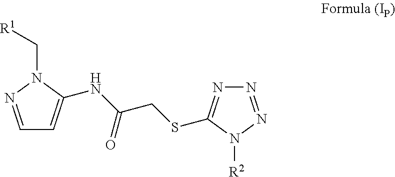

wherein
R¹ represents aryl or heteroaryl, wherein said aryl or heteroaryl independently is unsubstituted, or mono-, di-, or tri-substituted, wherein the substituents are independently selected from the group consisting of (C₁₋₄)alkyl, (C₁₋₄)alkoxy, fluoroalkyl, fluoroalkoxy, halogen, and N(CH₃)₂; and
R² represents phenyl which is unsubstituted; or phenyl which is mono-substituted, wherein the substituent is attached to position 2 or 3 of the phenyl ring, wherein the substituent is selected from the group consisting of (C₁₋₄)alkyl, (C₁₋₄)alkoxy, and halogen; or R² represents phenyl which is di-substituted, wherein the substituents are independently selected from the group consisting of (C₁₋₄)alkyl, (C₁₋₄)alkoxy, and halogen, wherein one substituent is attached to position 2 of the phenyl ring;
with the exception of the following compounds:
N-(2-Benzyl-2H-pyrazol-3-yl)-2-[1-(2,5-dimethyl-phenyl)-1H-tetrazol-5-ylsulfanyl]-acetamide (CAS Registry No. 877976-75-5);
2-[1-(2,5-Dimethyl-phenyl)-1H-tetrazol-5-ylsulfanyl]-N-(2-thiophen-2-ylmethyl-2H-pyrazol-3-yl)-acetamide (CAS Registry No. 956726-62-8); and
N-[2-(3-Chloro-benzyl)-2H-pyrazol-3-yl]-2-(1-phenyl-1H-tetrazol-5-ylsulfanyl)-acetamide (CAS Registry No. 1134681-37-0).

The compounds of formulae (I), (I_{C}), or (I_{P}) may contain one or more stereogenic or asymmetric centers, such as one or more asymmetric carbon atoms. The compounds of formulae (I), (I_{C}), or (I_{P}) may thus be present as mixtures of stereoisomers or preferably as pure stereoisomers. Mixtures of stereoisomers may be separated in a manner known to a person skilled in the art.

The term “halogen” means fluorine, chlorine, or bromine, preferably fluorine or chlorine.

The term “alkyl”, used alone or in combination, refers to a saturated straight or branched chain alkyl group containing one to four carbon atoms. The term “(C_{x-y})alkyl” (x and y each being an integer), refers to an alkyl group as defined before containing x to y carbon atoms. For example a (C₁₋₄)alkyl group contains from one to four carbon atoms. Examples of (C₁₋₄)alkyl groups are methyl, ethyl, propyl, isopropyl, n-butyl, isobutyl, sec.-butyl and tert.-butyl. Preferred are methyl and ethyl. In addition, for the substituent R¹, isopropyl is also a preferred example.

The term “alkoxy”, used alone or in combination, refers to an alkyl-O— group wherein the alkyl group is as defined before. The term “(C_{x-y})alkoxy” (x and y each being an integer) refers to an alkoxy group as defined before containing x to y carbon atoms. For example a (C₁₋₄)alkoxy group means a group of the formula (C₁₋₄)alkyl-O— in which the term “(C₁₋₄)alkyl” has the previously given significance. Examples of (C₁₋₄)alkoxy groups are methoxy, ethoxy, n-propoxy, isopropoxy, n-butoxy, isobutoxy, sec.-butoxy and tert.-butoxy. Preferred is methoxy. In addition, for the substituent R¹, ethoxy is also a preferred example.

The term “fluoroalkyl” refers to an alkyl group as defined before containing one to three carbon atoms in which one or more (and possibly all) hydrogen atoms have been replaced with fluorine. The term “(C_{x-y})fluoroalkyl” (x and y each being an integer) refers to a fluoroalkyl group as defined before containing x to y carbon atoms. For example a (C₁₋₃)fluoroalkyl group contains from one to three carbon atoms in which one to seven hydrogen atoms have been replaced with fluorine. Representative examples of fluoroalkyl groups include trifluoromethyl and 2,2,2-trifluoroethyl. Preferred are (C₁)fluoroalkyl groups such as trifluoromethyl.

The term “fluoroalkoxy” refers to an alkoxy group as defined before containing one to three carbon atoms in which one or more (and possibly all) hydrogen atoms have been replaced with fluorine. The term “(C_{x-y})fluoroalkoxy” (x and y each being an integer) refers to a fluoroalkoxy group as defined before containing x to y carbon atoms. For example a (C₁₋₃)fluoroalkoxy group contains from one to three carbon atoms in which one to seven hydrogen atoms have been replaced with fluorine. Representative examples of fluoroalkoxy groups include trifluoromethoxy, difluoromethoxy and 2,2,2-trifluoroethoxy. Preferred are (C₁)fluoroalkoxy groups such as trifluoromethoxy and difluoromethoxy. Most preferred is trifluoromethoxy.

The term “aryl” means a phenyl, a naphthyl, a 2,3-dihydro-benzofuranyl-, a benzo[1,3]dioxolyl-, a 2,3-dihydro-benzo[1,4]dioxinyl-, or a 4H-benzo[1,3]dioxinyl group. The aryl group is unsubstituted, or mono-, di-, or tri-substituted; wherein the substituents are independently selected from the group consisting of (C₁₋₄)alkyl, (C₁₋₄)alkoxy, fluoroalkyl, fluoroalkoxy, halogen, and N(CH₃)₂. 2,3-Dihydro-benzofuranyl-, benzo[1,3]dioxolyl-, 2,3-dihydro-benzo[1,4]dioxinyl- and 4H-benzo[1,3]dioxinyl groups are preferably unsubstituted.

“R¹” representing “aryl” preferably means phenyl (preferred) or naphthyl, which is independently unsubstituted, or mono-, di-, or tri-substituted; wherein the substituents are independently selected from the group consisting of (C₁₋₄)alkyl, (C₁₋₄)alkoxy, fluoroalkyl, fluoroalkoxy, halogen, and N(CH₃)₂ (especially methyl, methoxy, fluoro, chloro, bromo, trifluoromethyl, trifluoromethoxy, and N(CH₃)₂). Additionally, in another particular embodiment, R¹ representing “aryl” also means 2,3-dihydro-benzofuranyl; benzo[1,3]dioxolyl; 2,3-dihydro-benzo[1,4]dioxinyl; or 4H-benzo[1,3]dioxinyl (notably 2,3-dihydro-benzofuranyl and especially benzo[1,3]dioxolyl). Examples of R¹ representing “aryl” are preferably groups wherein aryl represents phenyl such as phenyl, 4-methylphenyl, 3-methylphenyl, 2-methylphenyl, 4-isopropylphenyl, 4-tert.-butylphenyl, 4-methoxyphenyl, 3-methoxyphenyl, 2-methoxyphenyl, 2,4-dimethoxyphenyl, 3,4-dimethoxyphenyl, 2,3,4-trimethoxyphenyl, 2,4,5-trimethoxyphenyl, 2,4,6-trimethoxyphenyl, 3,4,5-trimethoxyphenyl, 4-ethoxyphenyl, 4-n-propoxyphenyl, 4-n-butoxyphenyl, 4-isopropoxyphenyl, 4-methoxy-3-methylphenyl, 4-methoxy-2,3-dimethylphenyl, 4-methoxy-2,5-dimethylphenyl, 4-chlorophenyl, 3-chlorophenyl, 2-chlorophenyl, 3,4-dichlorophenyl, 3-chloro-4-methylphenyl, 4-fluorophenyl, 3-fluorophenyl, 2-fluorophenyl, 3-fluoro-4-methoxyphenyl, 4-trifluoromethoxyphenyl, 4-trifluoromethylphenyl, 3-trifluoromethylphenyl, 2-trifluoromethylphenyl, and 4-dimethylaminophenyl. In addition to the above listed groups, further examples are 3-fluoro-4-trifluoromethoxyphenyl, 2,5-difluoro-4-methoxyphenyl, 2,4-difluoro-3-methoxyphenyl, 4-benzyloxy-phenyl, 4-phenoxy-phenyl, and 3′-fluoro-3-biphenyl. In another embodiment, in addition to the above-listed examples, further examples of R¹ representing “aryl” are those wherein aryl does not represent phenyl such as benzo[1,3]dioxol-5-yl, and naphthyl (notably 2-naphthyl), and additionally 2,3-dihydro-benzofuranyl (notably 2,3-dihydro-benzofuran-5-yl). Preferred examples of R¹ representing “aryl” are 4-isopropylphenyl, 4-methoxyphenyl, 4-ethoxyphenyl, 4-methylphenyl, 4-methoxy-3-methylphenyl, 3-fluoro-4-methoxyphenyl, and 4-dimethylaminophenyl. In a sub-embodiment, preferred examples of R¹ representing “aryl” are 4-isopropylphenyl, 4-methoxyphenyl, 4-ethoxyphenyl, 4-methoxy-3-methylphenyl, 3-fluoro-4-methoxyphenyl, and 4-dimethylaminophenyl.

Examples of R² representing phenyl which is unsubstituted or substituted as explicitly described are 2-methylphenyl, 3-methylphenyl, 2-ethylphenyl, 2-methoxyphenyl, 3-methoxyphenyl, 2-fluorophenyl and 3-fluorophenyl. Further examples are 2,3-dimethylphenyl, 2,4-dimethylphenyl, 2,5-dimethylphenyl, 2,6-dimethylphenyl, 2-ethyl-6-methylphenyl, 2-methoxy-5-methylphenyl, 2,4-dimethoxyphenyl, and 2,5-dimethoxyphenyl. Further examples are phenyl, 3-chlorophenyl, 2-chlorophenyl, 4-methylphenyl, 2-chloro-6-methylphenyl, 3-fluoro-6-methylphenyl, 3-fluoro-2-methylphenyl, 2-fluoro-4-methylphenyl, 2-fluoro-5-methylphenyl, 3,5-dimethylphenyl, 2,3-dichlorophenyl, 2,5-dichlorophenyl, 2,6-dichlorophenyl, 2,4-difluorophenyl, 2,6-difluorophenyl, 2,3,4-trifluorophenyl, 2,3,6-trifluorophenyl, 2,4,6-trimethylphenyl, 2,6-diethylphenyl, 2,6-dimethoxyphenyl, 2-chloro-6-trifluoromethyl-phenyl, 2-trifluoromethoxyphenyl, and 2,6-diisopropylphenyl. Preferred are 2-methylphenyl, 3-methylphenyl, 2-trifluoromethoxyphenyl, 2,3-dimethylphenyl, 2,5-dimethylphenyl, 2,6-dimethylphenyl, 2-ethyl-6-methylphenyl, 2,6-diethylphenyl, 2-chloro-6-methylphenyl, 3,5-dimethylphenyl, 2,6-dichlorophenyl, 2-chloro-6-trifluoromethyl-phenyl, and 2,4,6-trimethylphenyl. In case “R²” represents “phenyl which is mono-substituted, wherein the substituent is attached to position 2 or 3 of the phenyl ring, wherein the substituent is selected from the group consisting of (C₁₋₄)alkyl, (C₁₋₄)alkoxy, and halogen”, the substituent is preferably selected from methyl, ethyl, methoxy and fluorine; especially the substituent is methyl. Examples of such mono-substituted phenyl groups as used for the substituent R² are 2-methylphenyl, 3-methylphenyl, 2-ethylphenyl, 2-methoxyphenyl, 3-methoxyphenyl, 2-fluorophenyl and 3-fluorophenyl. Preferred are 2-methylphenyl, and 3-methylphenyl. In case “R²” represents “phenyl which is di-substituted, wherein the substituents are independently selected from the group consisting of (C₁₋₄)alkyl, (C₁₋₄)alkoxy, and halogen, wherein one substituent is attached to position 2 of the phenyl ring”, the substituent in position 2 is preferably selected from (C₁₋₄)alkyl. Examples of such di-substituted phenyl groups as used for the substituent R² are 2,3-dimethylphenyl, 2,4-dimethylphenyl, 2,5-dimethylphenyl, 2,6-dimethylphenyl, 2-ethyl-6-methylphenyl, 2-methoxy-5-methylphenyl, 2,4-dimethoxyphenyl, and 2,5-dimethoxyphenyl. Preferred are 2,3-dimethylphenyl, 2,5-dimethylphenyl, 2,6-dimethylphenyl, and 2-ethyl-6-methylphenyl.

The term “heteroaryl” means a 5- to 10-membered monocyclic or bicyclic aromatic ring containing 1, 2 or 3 heteroatoms, each independently selected from oxygen, nitrogen and sulfur. Examples of such heteroaryl groups are furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidyl, pyridazinyl, pyrazinyl, indolyl, isoindolyl, benzofuranyl, isobenzofuranyl, benzothiophenyl, indazolyl, benzimidazolyl, benzoxazolyl, benzisoxazolyl, benzothiazolyl, benzoisothiazolyl, benzotriazolyl, benzoxadiazolyl, benzothiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidyl, imidazo[1,2-a]pyridyl and imidazo[2,1-b]thiazolyl. In case “R¹” represents “heteroaryl”, preferred examples are furanyl, thienyl, pyridyl, and indolyl. The above-mentioned heteroaryl groups are unsubstituted, mono-, di-, or tri-substituted (preferred unsubstituted, mono-, or di-substituted), wherein the substituents are independently selected from the group consisting of (C₁₋₄)alkyl, (C₁₋₄)alkoxy, fluoroalkyl, fluoroalkoxy, halogen, and N(CH₃)₂ (preferred (C₁₋₄)alkyl, (C₁₋₄)alkoxy, halogen, and trifluoromethyl; most preferred (C₁₋₄)alkyl, and (C₁₋₄)alkoxy). In particular, for the substituent “R¹”, thienyl, and indolyl groups are preferably unsubstituted; furanyl groups are preferably di-substituted with methyl; pyridyl groups are preferably unsubstituted or mono-substituted with methoxy. Examples of R¹ representing “heteroaryl” are 2,3-dimethyl-furan-5-yl, thiophen-2-yl, thiophen-3-yl, pyridin-2-yl, pyridin-3-yl, pyridin-4-yl, 2-methoxy-pyridin-5-yl, and indol-6-yl; and in addition to the above-listed groups 7-chloro-quinolin-4-yl and notably 5-methoxy-pyridin-3-yl.

Further embodiments of the invention are presented hereinafter:

16) A further embodiment of the invention relates to compounds of formula (I) according to any one of embodiments 1) to 15), wherein R¹ represents phenyl (preferred) or naphthyl, wherein said phenyl or naphthyl is independently unsubstituted, or mono-, di-, or tri-substituted; wherein the substituents are independently selected from the group consisting of (C₁₋₄)alkyl, (C₁₋₄)alkoxy, fluoroalkyl, fluoroalkoxy, halogen, and N(CH₃)₂; or R¹ represents a group selected from the group consisting of 2,3-dihydro-benzofuranyl, benzo[1,3]dioxolyl (preferred), 2,3-dihydro-benzo[1,4]dioxinyl, and 4H-benzo[1,3]dioxinyl; or R¹ represents heteroaryl, wherein said heteroaryl is unsubstituted, or mono-, or di-substituted; wherein the substituents are independently selected from the group consisting of (C₁₋₄)alkyl, (C₁₋₄)alkoxy, halogen, and trifluoromethyl (notably (C₁₋₄)alkyl, and (C₁₋₄)alkoxy).

17) Another embodiment relates to compounds according to any one of embodiments 1) to 15), wherein R¹ represents aryl, wherein said aryl is unsubstituted, or mono-, di-, or tri-substituted, wherein the substituents are independently selected from the group consisting of (C₁₋₄)alkyl, (C₁₋₄)alkoxy, fluoroalkyl, fluoroalkoxy, halogen, and N(CH₃)₂.

18) Another embodiment relates to compounds according to any one of embodiments 1) to 17), wherein R¹ represents phenyl, which is unsubstituted, or mono-, di-, or tri-substituted; wherein the substituents are independently selected from the group consisting of (C₁₋₄)alkyl, (C₁₋₄)alkoxy, fluoroalkyl, fluoroalkoxy, halogen, and N(CH₃)₂.

19) Another embodiment relates to compounds according to any one of embodiments 1) to 18), wherein R¹ represents 4-isopropylphenyl, 4-methoxyphenyl, 4-ethoxyphenyl, 4-methoxy-3-methylphenyl, 3-fluoro-4-methoxyphenyl, or 4-dimethylaminophenyl.

20) Another embodiment relates to compounds according to any one of embodiments 1) to 15), wherein R¹ represents heteroaryl, wherein said heteroaryl is unsubstituted, or mono-, or di-substituted; wherein the substituents are independently selected from the group consisting of (C₁₋₄)alkyl, (C₁₋₄)alkoxy, halogen, and trifluoromethyl (notably (C₁₋₄)alkyl, and (C₁₋₄)alkoxy).

21) Another embodiment relates to compounds according to any one of embodiments 1) to 20), wherein R² represents phenyl which is mono-substituted, wherein the substituent is attached to position 2 or 3 of the phenyl ring, wherein the substituent is selected from the group consisting of (C₁₋₄)alkyl, (C₁₋₄)alkoxy, and halogen; or R² represents phenyl which is di- or tri-substituted (notably di-substituted), wherein the substituents are independently selected from the group consisting of (C₁₋₄)alkyl, (C₁₋₄)alkoxy, halogen, fluoroalkyl, and fluoroalkoxy (notably (C₁₋₄)alkyl, (C₁₋₄)alkoxy, and halogen), wherein one substituent is attached to position 2 of the phenyl ring (it being understood that the present embodiment relates to embodiment 15) mutatis mutandis).

22) Another embodiment relates to compounds according to any one of embodiments 1) to 21), wherein R² represents phenyl which is mono-, di-, or tri-substituted (notably mono, or di-substituted), wherein the substituents are independently selected from the group consisting of (C₁₋₄)alkyl, (C₁₋₄)alkoxy, halogen, fluoroalkyl, and fluoroalkoxy (notably (C₁₋₄)alkyl, (C₁₋₄)alkoxy, and halogen), wherein one substituent is attached to position 2 of the phenyl ring (it being understood that the present embodiment relates to embodiment 15) mutatis mutandis).

23) Another embodiment relates to compounds according to any one of embodiments 1) to 22), wherein R² represents a phenyl group which is mono-substituted as explicitly defined before.

24) Another embodiment relates to compounds according to any one of embodiments 1) to 22), wherein R² represents a phenyl group which is di- or tri-substituted (notably di-substituted) as explicitly defined before (it being understood that the present embodiment relates to embodiment 15) mutatis mutandis).

25) Examples of compounds of formula (I) according to embodiment 1) are selected from the group consisting of:
2-[1-(2,3-Dimethyl-phenyl)-1H-tetrazol-5-ylsulfanyl]-N-[2-(4-methoxy-benzyl)-2H-pyrazol-3-yl]-acetamide;
2-[1-(2,3-Dimethyl-phenyl)-1H-tetrazol-5-ylsulfanyl]-N-[2-(4-methoxy-3-methyl-benzyl)-2H-pyrazol-3-yl]-acetamide;
2-[1-(2,3-Dimethyl-phenyl)-1H-tetrazol-5-ylsulfanyl]-N-[2-(3-fluoro-4-methoxy-benzyl)-2H-pyrazol-3-yl]-acetamide;
2-[1-(2,3-Dimethyl-phenyl)-1H-tetrazol-5-ylsulfanyl]-N-[2-(4-isopropyl-benzyl)-2H-pyrazol-3-yl]-acetamide;
2-[1-(2,3-Dimethyl-phenyl)-1H-tetrazol-5-ylsulfanyl]-N-[2-(4-methyl-benzyl)-2H-pyrazol-3-yl]-acetamide;
N-[2-(3,4-Dimethoxy-benzyl)-2H-pyrazol-3-yl]-2-[1-(2,3-dimethyl-phenyl)-1H-tetrazol-5-ylsulfanyl]-acetamide;
2-[1-(2,3-Dimethyl-phenyl)-1H-tetrazol-5-ylsulfanyl]-N-[2-(4-trifluoromethoxy-benzyl)-2H-pyrazol-3-yl]-acetamide;
2-[1-(2,3-Dimethyl-phenyl)-1H-tetrazol-5-ylsulfanyl]-N-[2-(4-trifluoromethyl-benzyl)-2H-pyrazol-3-yl]-acetamide;
2-[1-(2,3-Dimethyl-phenyl)-1H-tetrazol-5-ylsulfanyl]-N-[2-(4-ethoxy-benzyl)-2H-pyrazol-3-yl]-acetamide;
2-[1-(2,3-Dimethyl-phenyl)-1H-tetrazol-5-ylsulfanyl]-N-[2-(2-methyl-benzyl)-2H-pyrazol-3-yl]-acetamide;
N-[2-(3-Chloro-4-methyl-benzyl)-2H-pyrazol-3-yl]-2-[1-(2,3-dimethyl-phenyl)-1H-tetrazol-5-ylsulfanyl]-acetamide;
N-[2-(4-tert-Butyl-benzyl)-2H-pyrazol-3-yl]-2-[1-(2,3-dimethyl-phenyl)-1H-tetrazol-5-ylsulfanyl]-acetamide;
2-[1-(2,3-Dimethyl-phenyl)-1H-tetrazol-5-ylsulfanyl]-N-[2-(6-methoxy-pyridin-3-ylmethyl)-2H-pyrazol-3-yl]-acetamide;
2-[1-(2,3-Dimethyl-phenyl)-1H-tetrazol-5-ylsulfanyl]-N-(2-pyridin-4-ylmethyl-2H-pyrazol-3-yl)-acetamide;
N-[2-(4-Dimethylamino-benzyl)-2H-pyrazol-3-yl]-2-[1-(2,3-dimethyl-phenyl)-1H-tetrazol-5-ylsulfanyl]-acetamide;
2-[1-(2,3-Dimethyl-phenyl)-1H-tetrazol-5-ylsulfanyl]-N-(2-thiophen-3-ylmethyl-2H-pyrazol-3-yl)-acetamide;
2-[1-(2,3-Dimethyl-phenyl)-1H-tetrazol-5-ylsulfanyl]-N-(2-thiophen-2-ylmethyl-2H-pyrazol-3-yl)-acetamide;
N-[2-(4-M ethoxy-benzyl)-2H-pyrazol-3-yl]-2-(1-o-tolyl-1H-tetrazol-5-ylsulfanyl)-acetamide;
2-[1-(2,4-Dimethyl-phenyl)-1H-tetrazol-5-ylsulfanyl]-N-[2-(4-methoxy-benzyl)-2H-pyrazol-3-yl]-acetamide;
N-[2-(4-Methoxy-benzyl)-2H-pyrazol-3-yl]-2-[1-(2-methoxy-phenyl)-1H-tetrazol-5-ylsulfanyl]-acetamide;
N-[2-(4-Methoxy-benzyl)-2H-pyrazol-3-yl]-2-[1-(3-methoxy-phenyl)-1H-tetrazol-5-ylsulfanyl]-acetamide;
N-[2-(4-Methoxy-benzyl)-2H-pyrazol-3-yl]-2-(1-m-tolyl-1H-tetrazol-5-ylsulfanyl)-acetamide; and
2-[1-(2,5-Dimethyl-phenyl)-1H-tetrazol-5-ylsulfanyl]-N-[2-(4-ethoxy-benzyl)-2H-pyrazol-3-yl]-acetamide.

26) In addition to the compounds listed in embodiment 25), further examples of compounds of formula (I) according to embodiment 1) are selected from the group consisting of:
2-[1-(2,5-Dimethyl-phenyl)-1H-tetrazol-5-ylsulfanyl]-N-[2-(4-methoxy-benzyl)-2H-pyrazol-3-yl]-acetamide;
2-[1-(2,6-Dimethyl-phenyl)-1H-tetrazol-5-ylsulfanyl]-N-[2-(4-methoxy-benzyl)-2H-pyrazol-3-yl]-acetamide;
2-[1-(2-Ethyl-6-methyl-phenyl)-1H-tetrazol-5-ylsulfanyl]-N-[2-(4-methoxy-benzyl)-2H-pyrazol-3-yl]-acetamide; and
2-[1-(2,5-Dimethyl-phenyl)-1H-tetrazol-5-ylsulfanyl]-N-[2-(3-fluoro-4-methoxy-benzyl)-2H-pyrazol-3-yl]-acetamide.

27) In addition to the compounds listed in embodiments 25) and 26), further examples of compounds of formula (I) according to embodiment 1) are selected from the group consisting of:
2-[1-(2,3-Dimethyl-phenyl)-1H-tetrazol-5-ylsulfanyl]-N-[2-(4-isopropoxy-benzyl)-2H-pyrazol-3-yl]-acetamide;
2-[1-(2,3-Dimethyl-phenyl)-1H-tetrazol-5-ylsulfanyl]-N-[2-(4-propoxy-benzyl)-2H-pyrazol-3-yl]-acetamide;
N-[2-(4-Chloro-benzyl)-2H-pyrazol-3-yl]-2-[1-(2,3-dimethyl-phenyl)-1H-tetrazol-5-ylsulfanyl]-acetamide;
N-[2-(4-Methoxy-benzyl)-2H-pyrazol-3-yl]-2-[1-(2-methoxy-5-methyl-phenyl)-1H-tetrazol-5-ylsulfanyl]-acetamide;
2-[1-(2-Fluoro-phenyl)-1H-tetrazol-5-ylsulfanyl]-N-[2-(4-methoxy-benzyl)-2H-pyrazol-3-yl]-acetamide;
2-[1-(2,5-Dimethyl-phenyl)-1H-tetrazol-5-ylsulfanyl]-N-[2-(4-isopropyl-benzyl)-2H-pyrazol-3-yl]-acetamide;
2-[1-(2,5-Dimethyl-phenyl)-1H-tetrazol-5-ylsulfanyl]-N-[2-(4-methyl-benzyl)-2H-pyrazol-3-yl]-acetamide; and
2-[1-(2,5-Dimethyl-phenyl)-1H-tetrazol-5-ylsulfanyl]-N-[2-(4-methoxy-3-methyl-benzyl)-2H-pyrazol-3-yl]-acetamide.

28) In addition to the compounds listed in embodiment 25), 26), and 27), further examples of compounds of formula (I) according to embodiment 1) are selected from the group consisting of:
2-[1-(2-Chloro-phenyl)-1H-tetrazol-5-ylsulfanyl]-N-[2-(4-methoxy-benzyl)-2H-pyrazol-3-yl]-acetamide;
2-[1-(2,5-Dichloro-phenyl)-1H-tetrazol-5-ylsulfanyl]-N-[2-(4-methoxy-benzyl)-2H-pyrazol-3-yl]-acetamide;
2-[1-(3,5-Dimethyl-phenyl)-1H-tetrazol-5-ylsulfanyl]-N-[2-(4-methoxy-benzyl)-2H-pyrazol-3-yl]-acetamide;
2-[1-(3-Chloro-phenyl)-1H-tetrazol-5-ylsulfanyl]-N-[2-(4-methoxy-benzyl)-2H-pyrazol-3-yl]-acetamide;
2-[1-(2,3-Dimethyl-phenyl)-1H-tetrazol-5-ylsulfanyl]-N-(2-phenethyl-2H-pyrazol-3-yl)-acetamide;
2-[1-(2,3-Dimethyl-phenyl)-1H-tetrazol-5-ylsulfanyl]-N-[2-(5-methoxy-pyridin-3-ylmethyl)-2H-pyrazol-3-yl]-acetamide;
2-[1-(2,3-Dimethyl-phenyl)-1H-tetrazol-5-ylsulfanyl]-N-[2-(3-phenyl-propyl)-2H-pyrazol-3-yl]-acetamide;
2-[1-(2,3-Dimethyl-phenyl)-1H-tetrazol-5-ylsulfanyl]-N-[2-(4-phenoxy-benzyl)-2H-pyrazol-3-yl]-acetamide;
N-[2-(4-Benzyloxy-benzyl)-2H-pyrazol-3-yl]-2-[1-(2,3-dimethyl-phenyl)-1H-tetrazol-5-ylsulfanyl]-acetamide;
N-{2-[3-(2,5-Difluoro-4-methoxy-phenyl)-propyl]-2H-pyrazol-3-yl}-2-[1-(2,3-dimethyl-phenyl)-1H-tetrazol-5-ylsulfanyl]-acetamide;
N-[2-(2,3-Dihydro-benzofuran-5-ylmethyl)-2H-pyrazol-3-yl]-2-[1-(2,6-dimethyl-phenyl)-1H-tetrazol-5-ylsulfanyl]-acetamide;
2-[1-(2,5-Dimethyl-phenyl)-1H-tetrazol-5-ylsulfanyl]-N-(2-phenethyl-2H-pyrazol-3-yl)-acetamide;
2-[1-(2,6-Dimethyl-phenyl)-1H-tetrazol-5-ylsulfanyl]-N-[2-(3-phenyl-propyl)-2H-pyrazol-3-yl]-acetamide;
2-[1-(2,6-Difluoro-phenyl)-1H-tetrazol-5-ylsulfanyl]-N-[2-(4-methoxy-benzyl)-2H-pyrazol-3-yl]-acetamide;
2-[1-(2,6-Diethyl-phenyl)-1H-tetrazol-5-ylsulfanyl]-N-[2-(4-methoxy-benzyl)-2H-pyrazol-3-yl]-acetamide;
2-[1-(2,6-Diisopropyl-phenyl)-1H-tetrazol-5-ylsulfanyl]-N-[2-(4-methoxy-benzyl)-2H-pyrazol-3-yl]-acetamide;
2-[1-(2,6-Dichloro-phenyl)-1H-tetrazol-5-ylsulfanyl]-N-[2-(4-methoxy-benzyl)-2H-pyrazol-3-yl]-acetamide;
2-[1-(2-Chloro-6-methyl-phenyl)-1H-tetrazol-5-ylsulfanyl]-N-[2-(4-methoxy-benzyl)-2H-pyrazol-3-yl]-acetamide;
N-[2-(4-Methoxy-benzyl)-2H-pyrazol-3-yl]-2-[1-(2,4,6-trimethyl-phenyl)-1H-tetrazol-5-ylsulfanyl]-acetamide;
2-[1-(2-Fluoro-5-methyl-phenyl)-1H-tetrazol-5-ylsulfanyl]-N-[2-(4-methoxy-benzyl)-2H-pyrazol-3-yl]-acetamide;
2-[1-(3-Fluoro-2-methyl-phenyl)-1H-tetrazol-5-ylsulfanyl]-N-[2-(4-methoxy-benzyl)-2H-pyrazol-3-yl]-acetamide;
N-[2-(4-Methoxy-benzyl)-2H-pyrazol-3-yl]-2-[1-(2,3,5-trifluoro-phenyl)-1H-tetrazol-5-ylsulfanyl]-acetamide;
2-[1-(5-Fluoro-2-methyl-phenyl)-1H-tetrazol-5-ylsulfanyl]-N-[2-(4-methoxy-benzyl)-2H-pyrazol-3-yl]-acetamide;
N-[2-(4-Methoxy-benzyl)-2H-pyrazol-3-yl]-2-[1-(2-trifluoromethoxy-phenyl)-1H-tetrazol-5-ylsulfanyl]-acetamide;
2-[1-(2,3-Dichloro-phenyl)-1H-tetrazol-5-ylsulfanyl]-N-[2-(4-methoxy-benzyl)-2H-pyrazol-3-yl]-acetamide;
3-[1-(2,3-Dimethyl-phenyl)-1H-tetrazol-5-yl]-N-[2-(4-methoxy-benzyl)-2H-pyrazol-3-yl]-propionamide;
3-[1-(2,5-Dimethyl-phenyl)-1H-tetrazol-5-yl]-N-[2-(4-methoxy-benzyl)-2H-pyrazol-3-yl]-propionamide;
3-[1-(2,6-Dimethyl-phenyl)-1H-tetrazol-5-yl]-N-[2-(4-methoxy-benzyl)-2H-pyrazol-3-yl]-propionamide;
N-[2-(4-Methoxy-benzyl)-2H-pyrazol-3-yl]-3-[1-(2,4,6-trimethyl-phenyl)-1H-tetrazol-5-yl]-propionamide;
N-[2-(4-Methoxy-benzyl)-2H-pyrazol-3-yl]-3-(1-naphthalen-1-yl-1H-tetrazol-5-yl)-propionamide;
3-[1-(2,6-Diethyl-phenyl)-1H-tetrazol-5-yl]-N-[2-(4-methoxy-benzyl)-2H-pyrazol-3-yl]-propionamide;
3-[1-(2,3-Dimethyl-phenyl)-1H-tetrazol-5-yl]-N-[2-(4-isopropyl-benzyl)-2H-pyrazol-3-yl]-propionamide;
3-[1-(2,5-Dimethyl-phenyl)-1H-tetrazol-5-yl]-N-[2-(4-isopropyl-benzyl)-2H-pyrazol-3-yl]-propionamide;
3-[1-(2,6-Dimethyl-phenyl)-1H-tetrazol-5-yl]-N-[2-(4-isopropyl-benzyl)-2H-pyrazol-3-yl]-propionamide;
3-[1-(2,6-Diethyl-phenyl)-1H-tetrazol-5-yl]-N-[2-(4-methoxy-benzyl)-5-methyl-2H-pyrazol-3-yl]-propionamide;
2-[1-(2,3-Dimethyl-phenyl)-1H-tetrazol-5-ylsulfanyl]-N-[2-(4-methoxy-benzyl)-5-methyl-2H-pyrazol-3-yl]-acetamide;
3-[1-(2,6-Diethyl-phenyl)-1H-tetrazol-5-yl]-N-[2-(4-methoxy-benzyl)-4-methyl-2H-pyrazol-3-yl]-propionamide;
2-[1-(2,5-Dimethyl-phenyl)-1H-tetrazol-5-ylsulfanyl]-N-[2-(4-methoxy-phenyl)-2H-pyrazol-3-yl]-acetamide;
2-[1-(2,3-Dimethyl-phenyl)-1H-tetrazol-5-ylsulfanyl]-N-[2-(4-isopropyl-phenyl)-2H-pyrazol-3-yl]-acetamide;
2-[1-(2,3-Dimethyl-phenyl)-1H-tetrazol-5-ylsulfanyl]-N-[2-(4-fluoro-phenyl)-2H-pyrazol-3-yl]-acetamide;
2-[1-(2,3-Dimethyl-phenyl)-1H-tetrazol-5-ylsulfanyl]-N-[2-(3-methoxy-phenyl)-2H-pyrazol-3-yl]-acetamide;
2-[1-(2,3-Dimethyl-phenyl)-1H-tetrazol-5-ylsulfanyl]-N-[2-(4-phenoxy-phenyl)-2H-pyrazol-3-yl]-acetamide;
2-[1-(2,3-Dimethyl-phenyl)-1H-tetrazol-5-ylsulfanyl]-N-(2-p-tolyl-2H-pyrazol-3-yl)-acetamide;
2-[1-(2,3-Dimethyl-phenyl)-1H-tetrazol-5-ylsulfanyl]-N-[2-(3-fluoro-4-methoxy-phenyl)-2H-pyrazol-3-yl]-acetamide;
2-[1-(2,3-Dimethyl-phenyl)-1H-tetrazol-5-ylsulfanyl]-N-[2-(6-methoxy-pyridin-3-yl)-2H-pyrazol-3-yl]-acetamide;
N-[2-(4-Methoxy-phenyl)-2H-pyrazol-3-yl]-2-[1-(2,4,6-trimethyl-phenyl)-1H-tetrazol-5-ylsulfanyl]-acetamide;
N-[2-(4-Methoxy-phenyl)-2H-pyrazol-3-yl]-2-(1-naphthalen-1-yl-1H-tetrazol-5-ylsulfanyl)-acetamide;
2-[1-(2,6-Dichloro-phenyl)-1H-tetrazol-5-ylsulfanyl]-N-[2-(4-methoxy-phenyl)-2H-pyrazol-3-yl]-acetamide;
2-[1-(2,6-Dimethyl-phenyl)-1H-tetrazol-5-ylsulfanyl]-N-[2-(4-methoxy-phenyl)-2H-pyrazol-3-yl]-acetamide.

Any reference to a compound of formulae (I), (I_{C}), or (I_{P}) is to be understood as referring also to the salts (and especially the pharmaceutically acceptable salts) of such compounds, as appropriate and expedient.

Where the plural form is used for compounds, salts, pharmaceutical compositions, diseases or the like, this is intended to mean also a single compound, salt, disease or the like.

The term “pharmaceutically acceptable salts” refers to non-toxic, inorganic or organic acid and/or base addition salts. Reference can be made to “Salt selection for basic drugs”, Int. J. Pharm. (1986), 33, 201-217.

The compounds of formula (I), (I_{C}), and (I_{P}) according to embodiments 1), 3) or 15), including the therein specifically excluded compounds, and their pharmaceutically acceptable salts can be used as medicaments, e.g. in the form of pharmaceutical compositions for enteral or parenteral administration.

A further aspect of the invention is a pharmaceutical composition containing at least one compound of formula (I), (I_{C}), or (I_{P}) according to embodiments 1), 3) or 15), including the therein specifically excluded compounds, or a pharmaceutically acceptable salt thereof, and a pharmaceutically acceptable carrier material.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula (I) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The present invention also relates to a method for the prevention or treatment of a disease or disorder mentioned herein comprising administering to a subject a pharmaceutically active amount of a compound of formulae (I), (I_{C}), or (I_{P}) according to embodiments 1), 3) or 15), including the therein specifically excluded compounds.

For avoidance of any doubt, if compounds are described as useful for the prevention or treatment of certain diseases, such compounds are likewise suitable for use in the preparation of a medicament for the prevention or treatment of said diseases.

The compounds of formulae (I), (I_{C}), and (I_{P}) according to embodiments 1), 3) or 15), including the therein specifically excluded compounds, are useful for the prevention or treatment of diseases related to the orexin system.

Such diseases related to the orexin system may be selected from the group consisting of dysthymic disorders including major depression and cyclothymia, affective neurosis, all types of manic depressive disorders, delirium, psychotic disorders, schizophrenia, catatonic schizophrenia, delusional paranoia, adjustment disorders and all clusters of personality disorders; schizoaffective disorders; anxiety disorders including generalized anxiety, obsessive compulsive disorder, posttraumatic stress disorder, panic attacks, all types of phobic anxiety and avoidance; separation anxiety; all psychoactive substance use, abuse, seeking and reinstatement; all types of psychological or physical addictions, dissociative disorders including multiple personality syndromes and psychogenic amnesias; sexual and reproductive dysfunction; psychosexual dysfunction and addiction; tolerance to narcotics or withdrawal from narcotics; increased anaesthetic risk, anaesthetic responsiveness; hypothalamic-adrenal dysfunctions; disturbed biological and circadian rhythms; sleep disturbances associated with diseases such as neurological disorders including neuropathic pain and restless leg syndrome; sleep apnea; narcolepsy; chronic fatigue syndrome; insomnias related to psychiatric disorders; all types of idiopathic insomnias and parasomnias; sleep-wake schedule disorders including jet-lag; all dementias and cognitive dysfunctions in the healthy population and in psychiatric and neurological disorders; mental dysfunctions of aging; all types of amnesia; severe mental retardation; dyskinesias and muscular diseases; muscle spasticity, tremors, movement disorders; spontaneous and medication-induced dyskinesias; neurodegenerative disorders including Huntington's, Creutzfeld-Jacob's, Alzheimer's diseases and Tourette syndrome; Amyotrophic lateral sclerosis; Parkinson's disease; Cushing's syndrome; traumatic lesions; spinal cord trauma; head trauma; perinatal hypoxia; hearing loss; tinnitus; demyelinating diseases; spinal and cranial nerve diseases; ocular damage; retinopathy; epilepsy; seizure disorders; absence seizures, complex partial and generalized seizures; Lennox-Gastaut syndrome; migraine and headache; pain disorders; anaesthesia and analgesia; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; dental pain; pain related to infection e.g. by HIV; post-chemotherapy pain; post-stroke pain; post-operative pain; neuralgia; osteoarthritis; conditions associated with visceral pain such as irritable bowel syndrome; eating disorders; diabetes; toxic and dysmetabolic disorders including cerebral anoxia, diabetic neuropathies and alcoholism; appetite, taste, eating, or drinking disorders; somatoform disorders including hypochondriasis; vomiting/nausea; emesis; gastric dyskinesia; gastric ulcers; Kallman's syndrome (anosmia); impaired glucose tolerance; intestinal motility dyskinesias; hypothalamic diseases; hypophysis diseases; hyperthermia syndromes, pyrexia, febrile seizures, idiopathic growth deficiency; dwarfism; gigantism; acromegaly; basophil adenoma; prolactinoma; hyperprolactinemia; brain tumors, adenomas; benign prostatic hypertrophy, prostate cancer; endometrial, breast, colon cancer; all types of testicular dysfunctions, fertility control; reproductive hormone abnormalities; hot flashes; hypothalamic hypogonadism, functional or psychogenic amenorrhea; urinary bladder incontinence; asthma; allergies; all types of dermatitis, acne and cysts, sebaceous gland dysfunctions; cardiovascular disorders; heart and lung diseases, acute and congestive heart failure; hypotension; hypertension; dyslipidemias, hyperlipidemias, insulin resistance; urinary retention; osteoporosis; angina pectoris; myocardial infarction; arrhythmias, coronary diseases, left ventricular hypertrophy; ischemic or haemorrhagic stroke; all types of cerebrovascular disorders including subarachnoid haemorrhage, ischemic and hemorrhagic stroke and vascular dementia; chronic renal failure and other renal diseases; gout; kidney cancer; urinary incontinence; and other diseases related to general orexin system dysfunctions.

In particular, such diseases related to the orexin system may be selected from the group consisting of all types of sleep disorders, of stress-related syndromes, of addictions (especially psychoactive substance use, abuse, seeking and reinstatement), of cognitive dysfunctions in the healthy population and in psychiatric and neurologic disorders, of eating or drinking disorders.

Eating disorders may be defined as comprising metabolic dysfunction; dysregulated appetite control; compulsive obesities; emeto-bulimia or anorexia nervosa. Pathologically modified food intake may result from disturbed appetite (attraction or aversion for food); altered energy balance (intake vs. expenditure); disturbed perception of food quality (high fat or carbohydrates, high palatability); disturbed food availability (unrestricted diet or deprivation) or disrupted water balance. Drinking disorders include polydipsias in psychiatric disorders and all other types of excessive fluid intake.

Sleep disorders include all types of parasomnias, insomnias, narcolepsy and other disorders of excessive sleepiness, sleep-related dystonias; restless leg syndrome; sleep apneas; jet-lag syndrome; shift-work syndrome, delayed or advanced sleep phase syndrome or insomnias related to psychiatric disorders.

Insomnias are defined as comprising sleep disorders associated with aging; intermittent treatment of chronic insomnia; situational transient insomnia (new environment, noise) or short-term insomnia due to stress; grief; pain or illness. Insomnia also include stress-related syndromes including post-traumatic stress disorders as well as other types and subtypes of anxiety disorders such as generalized anxiety, obsessive compulsive disorder, panic attacks and all types of phobic anxiety and avoidance.

Addictions may be defined as addiction to one or more rewarding stimuli, notably to one rewarding stimulus. Such rewarding stimuli may be of either natural or synthetic origin. Psychoactive substance use, abuse, seeking and reinstatement are defined as all types of psychological or physical addictions and their related tolerance and dependence components.

Cognitive dysfunctions include deficits in all types of attention, learning and memory functions occurring transiently or chronically in the normal, healthy, young, adult or aging population, and also occurring transiently or chronically in psychiatric, neurologic, cardiovascular and immune disorders.

In a sub-embodiment, such diseases related to the orexin system may be selected from the group consisting of sleep disorders that comprises all types of insomnias, narcolepsy and other disorders of excessive sleepiness, sleep-related dystonias, restless leg syndrome, sleep apneas, jet-lag syndrome, shift-work syndrome, delayed or advanced sleep phase syndrome or insomnias related to psychiatric disorders (notably all types of insomnias, especially primary insomnia).

In another sub-embodiment, such diseases related to the orexin system may be selected from the group consisting of cognitive dysfunctions that comprise deficits in all types of attention, learning and memory functions occurring transiently or chronically in the normal, healthy, young, adult or aging population, and also occurring transiently or chronically in psychiatric, neurologic, cardiovascular and immune disorders.

In another sub-embodiment, such diseases related to the orexin system may be selected from the group consisting of eating disorders that comprise metabolic dysfunction; dysregulated appetite control; compulsive obesities; emeto-bulimia or anorexia nervosa.

In another sub-embodiment, such diseases related to the orexin system may be selected from the group consisting of all types of addictions (especially psychoactive substance use, abuse, seeking and reinstatement) that comprise all types of psychological or physical addictions and their related tolerance and dependence components.

Besides, any characteristics described in this invention for the compounds of formula (I) (whether for the compounds themselves, salts thereof, compositions containing the compounds or salts thereof, uses of the compounds or salts thereof, etc.) apply mutatis mutandis to compounds of formula (I_{C}), and formula (I_{P}).

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C. Besides, the term “room temperature” (RT) as used herein refers to a temperature of about 25° C.

### Preparation of Compounds of Formula (I)

A further aspect of the invention is a process for the preparation of compounds of formula (I). Compounds according to formula (I) of the present invention can be prepared according to the general sequence of reactions outlined in the schemes below wherein R¹ and R² are as defined in the description for formula (I). The compounds obtained may also be converted into salts, especially pharmaceutically acceptable salts thereof in a manner known per se.

The compounds of formula (I) and may be prepared as described in Scheme 1 to Scheme 3. The preparation of the pyrazole building blocks 3, 7 and 9 are described in Scheme 1. Pyrazoles 3 can be synthesized by adding the respective aldehyde 1 in portions to a solution of 2-cyanoethylhydrazine (2, commercially available) in ethanol. After heating the resulting mixture to 70° C. for 2 hours and basic work-up (e.g. NaOtBu in i-PrOH at 110° C. as described in detail in the experimental section below) the 3-amino-pyrazole building blocks 3 are obtained. The preparation of pyrazoles 7, can be performed by refluxing commercially available 4-dimethylamino-1,1-dimethoxy-but-3-en-2-one 5 and commercially available hydrazine hydrochlorides or hydrazines 4 in EtOH for 2-18 hours. The obtained esters 6 may be hydrolysed and decarboxylated with 37% HCl at 90° C. for 18 hours or hydrolyzed under basic conditions (2N aq. NaOH soln. in MeOH at r.t. or 45° C.) followed by decarboxylation under acidic conditions (HCl, 37%) at 60 to 90° C. for 18 hours). Pyrazols 9 may be synthesized by addition of hydrazine to acrylonitirile 8 (commercially available), followed by addition of aldehyde 1 at r.t. for 2 hours.

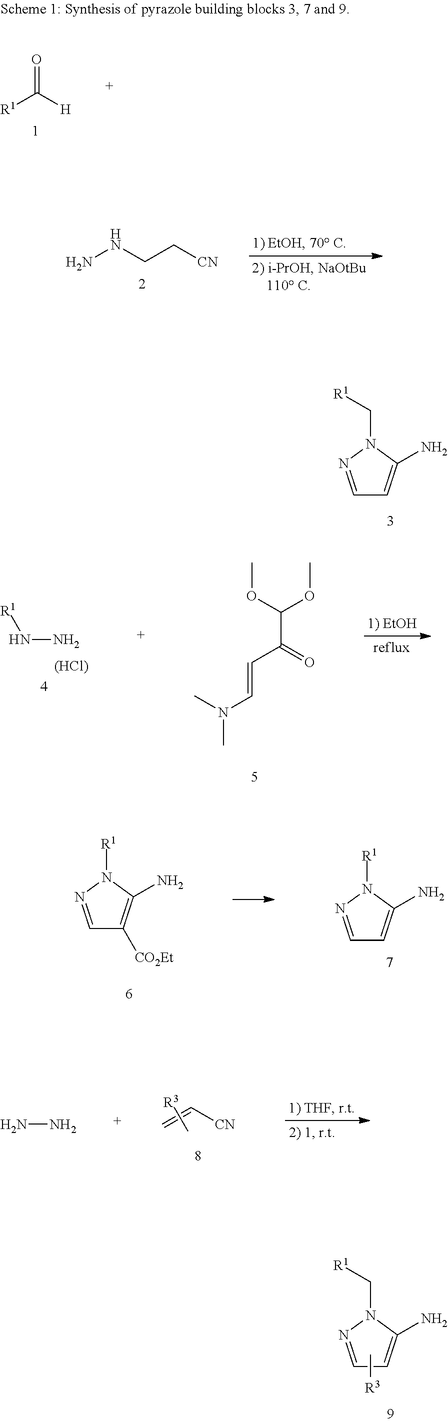

In Scheme 2, the synthesis of tetrazole building blocks 13 and 17 is described. The reaction of isothiocyanate-derivatives 10 (commercially available) with sodium azide (e.g. in EtOH at 70° C. for 2.5 hours) yields the tetrazole-derivatives 11. Alkylation of compounds II with ethyl bromoacetate (e.g. in DMSO in the presence of pyridine at r.t. for 2.5 hours) yields intermediates 12. Hydrolysis of the esters under standard reaction conditions (e.g. THF, MeOH, 1M NaOH, r.t. or 50° C.) yields the acids 13. Alternatively, reaction of amines 14 (commercially available) with 3-chlorocarbonyl-propionic acid ethyl ester 15 in the presence of DIPEA in DCM at r.t. yields intermediates 16, which may be cyclized in the presence of trimethylsilylazide under Mitsunobu conditions (DIAD, PPh₃ in THF, r.t.; WO2004/050643) followed by hydrolysis (1M aq. NaOH in THF/MeOH, r.t. for 18 hours) to carboxylic acids 17.

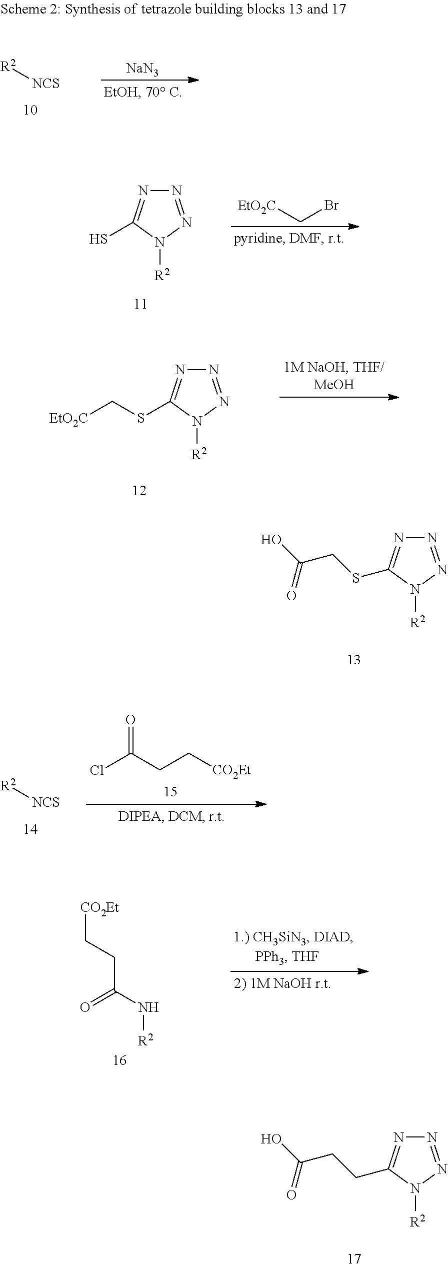

Pyrazoles 3, 7 or 9 can either be directly coupled with carboxylic acids 13 or 17 to yield compounds of formula (I) using standard amide coupling conditions (e.g. EDC, DMAP, DMF, rt, 48 hours or HATU, DIPEA, THF, rt, 4-24 hours) or they can be synthesized via acylation of the pyrazoles 3 or 9 to yield intermediates 18 (DMSO or DMF, pyridine, r.t.), followed by an alkylation of tetrazole 11 (DMF, pyridine, r.t.) (see Scheme 3).

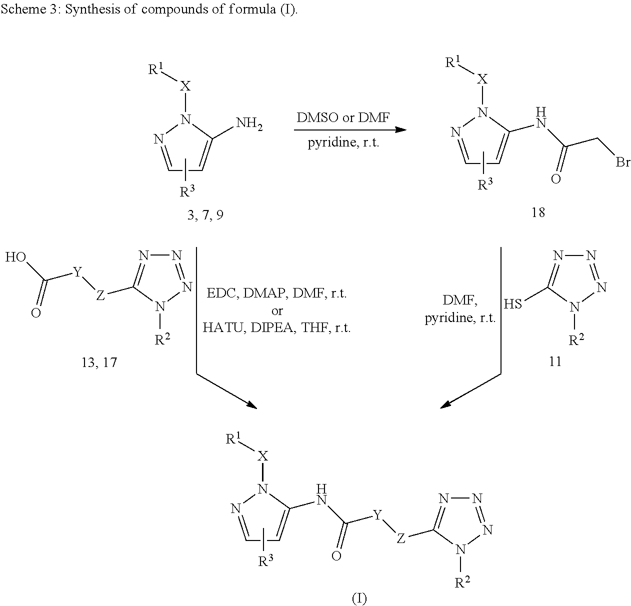

Aldehydes 1 are commercially available or readily prepared according to methods well known in the art e.g. from corresponding carboxylic acid derivatives or from corresponding aryl- or heteroaryl-halogenides (synthesis of 1 or precursors thereof in case R¹ represents heteroaryl: see for example T. Eicher, S. Hauptmann “The chemistry of Heterocycles: Structure, Reactions, Syntheses, and Applications”, 2nd Edition 2003, Wiley, ISBN 978-3-527-30720-3). Hydrazines 4 are commercially available or readily prepared according to methods well know in the art (e.g. from anilines, see WO2006/036994))

### EXPERIMENTAL SECTION

### Abbreviations

### As Used Herein and in the Description Above

AcOEt ethyl acetate
aq. aqueous
BSA Bovine serum albumine
CC column chromatography on silica gel
CHO Chinese hamster ovary
comb. combined
DABCO 1,4-diazabicyclo[2.2.2]octane
DCM dichloromethane
DIAD diisopropyl azodicarboxylate
DIPEA N-ethyldiisopropylamine
DMAP 4-dimethylamino-pyridine
DMF dimethylformamide
DMSO dimethylsulfoxide
EDC 1-ethyl-3-(3-dimethylaminopropyl)carbodiimide (HCl)
eq. equivalent
Et ethyl
Et₂O diethyl ether
EtOH ethanol
FCS Foatal calf serum
FLIPR Fluorescent imaging plate reader
HATU O-(7-Azabenzo-triazol-1-yl)-N,N,N′,N-tetra-methyluronium hexafluoro-phosphate
HBSS Hank's balanced salt solution
HEPES 4-(2-hydroxyethyl)-piperazine-1-ethanesulfonic acid
Hept heptane
HPLC high performance liquid chromatography
i-PrOH iso-propanol
LC liquid chromatography
M molarity [mol L⁻¹]
Me methyl
MeCN acetonitrile
MeOH methanol
MS mass spectroscopy
N normality
NaOtBu sodium tert. (tertiary) butoxide
org. organic
Ph phenyl (as in PPh₃=triphenylphosphin)
prep. preparative
r.t. room temperature
sat. saturated
soln. solution
THF tetrahydrofuran
UV ultraviolet

### I-Chemistry

The following examples illustrate the preparation of biologically active compounds of the invention but do not at all limit the scope thereof.

All temperatures are stated in ° C.

Compounds are characterized by:

LC-MS (A):

Agilent 1100 series with DAD, LSDS, and MS detection (MS: ESI⁺ and ESI⁻, AB SciexInstruments triple quadrupole); column: onyx monolithic C18 (100×3 mm); Conditions: acidic: eluent A: MeCN, eluent B: formic acid in water (0.05%), 10% to 90% MeCN, flow rate 1.8 mL/min; t_{R} is given in min;

LC-MS (A1):

Agilent 1100 series with DAD and MS detection (MS: Finnigan single quadrupole; columns (4.6×50 mm, 5 μm): Waters Atlantis T3, Waters Symmetry C18, Zorbax SB-AQ, or Waters Xbridge C18; Conditions: acidic: eluent A: MeCN, eluent B: TFA in water (0.4 mL/L), 5% to 95% MeCN, flow rate 4.5 mL/min; t_{R} is given in min;

LC-MS (A2):

Ultimate 3000 series with DAD and MS detection (MS: Finnigan single quadrupole); columns (4.6×50 mm, 5 μm): Waters Xbridge C18, Waters Atlantis T3, or Zorbax SB-AQ; Conditions: acidic: eluent A: MeCN, eluent B: TFA in water (0.4 mL/L), 5% to 95% MeCN, flow rate 4.5 mL/min; t_{R} is given in min;

LC-MS (B):

Agilent 1100 series with DAD and MS detection (MS: Finnigan single quadrupole; columns (4.6×50 mm, 5 μm): Zorbax SB-AQ, Zorbax Extend C18 or Waters Xbridge C18; Conditions: basic: eluent A: MeCN, eluent B: conc. NH₃ in water (1.0 mL/L), 5% to 95% MeCN, flow rate 4.5 mL/min; t_{R} is given in min; Compounds are purified by column chromatography on silica gel (CC) or by preparative HPLC using RP-C₁₈ based columns with MeCN/water gradients and ammonia or formic acid additives.

### Preparation of Examples

### Example 1

### 2-[1-(2,3-Dimethyl-phenyl)-1H-tetrazol-5-ylsulfanyl]-N-[2-(4-methoxy-benzyl)-2H-pyrazol-3-yl]-acetamide

### Step 1: 1-(2,3-Dimethyl-phenyl)-1H-tetrazole-5-thiol

To a solution of 2,3-dimethylphenyl isothiocyanate (1.63 g, 10.0 mmol, 1 eq.) in EtOH (400 mL), NaN₃ (9.75 g, 150.0 mmol, 15 eq.) was added. The mixture was stirred at 70° C. for 2.5 hours. The mixture was allowed to cool to r.t. and 37% HCl (4.2 mL) was carefully added. The resulting suspension was concentrated in vacuo. The residue was suspended in AcOEt (150 mL) and the mixture was extracted with 1M aq. NaOH solution (2×100 mL). The comb. aq. phases were carefully acidified with 6N HCl (50 mL). The resulting suspension was stored at 4° C. for 2 hours, then filtered. The solids were washed with water (5 mL) and dried under vacuo to give the desired tetrazole as an off-white solid. The product was used without further purification.

LC-MS (A): t_{R}=2.83 min; [M+H]⁺=207.0.

### Step 2: [1-(2,3-Dimethyl-phenyl)-1H-tetrazol-5-ylsulfanyl]-acetic acid ethyl ester

To a solution of 1-(2,3-dimethyl-phenyl)-1H-tetrazole-5-thiol (1.60 g, 7.76 mmol) in DMSO (20 mL), pyridine (0.78 ml, 9.70 mmol, 1.25 eq.) and ethyl bromoacetate (0.86 ml, 7.76 mmol, 1 eq.) were added in sequence. The resulting solution was stirred at r.t. for 2.5 hours. The pale yellow solution was diluted with AcOEt (100 mL) and washed successively with water (1×100 mL) and sat. aq. NaCl soln. (1×100 mL). The org. layer was dried over MgSO₄ and concentrated in vacuo. The residue was purified by CC (Hept/AcOEt 6/4) to yield the desired ester as a colorless oil.

LC-MS (A): t_{R}=3.21 min; [M+H]⁺=293.2.

### Step 3: [1-(2,3-Dimethyl-phenyl)-1H-tetrazol-5-ylsulfanyl]-acetic acid

To a solution of [1-(2,3-dimethyl-phenyl)-1H-tetrazol-5-ylsulfanyl]-acetic acid ethyl ester (2.14 g, 7.32 mmol) in THF (20.8 mL) and MeOH (6.2 mL), 1M aq. NaOH (6.2 mL) solution was added. The solution was stirred at r.t. overnight. The solution was concentrated in vacuo. The residue was dissolved in 1M aq. NaOH solution (20 mL) and water (20 mL). The solution was acidified with 1N aq. HCl solution to pH=3. The resulting suspension was stored at 4° C. during 1 h30 then filtered. The solids were dried under vacuum to give the desired acid as a white solid. The product was used without further purification.

LC-MS (A): t_{R}=2.80 min; [M+H]⁺=265.4.

### Step 4: 2-(4-Methoxy-benzyl)-2H-pyrazol-3-ylamine

To a solution of 2-cyanoethylhydrazine (0.81 ml, 10.0 mmol, 1 eq.) in EtOH (5 mL), anisaldehyde (1.21 ml, 10.0 mmol, 1 eq.) was added. The orange solution was stirred at 70° C. for 2 hours. The orange solution was allowed to cool to r.t. and concentrated in vacuo. The residue was dissolved in i-PrOH (8 mL). NaOtBu (991 mg, 10.0 mmol, 1 eq.) was added and the mixture was stirred at 110° C. for 4 hours. The mixture was allowed to cool to r.t. and water (50 mL) was added. The mixture was extracted with Et₂O (3×50 mL). The comb. org. phases were extracted with 1N aq. HCl (2×30 mL). The comb. aq. layers were basified to pH 14 with 50% aq. NaOH solution, then extracted with Et₂O (3×50 mL). The comb. org. phases were dried over MgSO₄ and concentrated in vacuo to give the desired pyrazole as an orange solid. The product was used without further purification.

LC-MS (A): t_{R}=1.26 min; [M+H]⁺=204.20.

### Step 5: Title Compound

To a solution of [1-(2,3-dimethyl-phenyl)-1H-tetrazol-5-ylsulfanyl]-acetic acid (529 mg, 2.0 mmol, 1.0 eq.) and 2-(4-methoxy-benzyl)-2H-pyrazol-3-ylamine (406 mg, 2.0 mmol, 1.0 eq.) in DMF (10 mL), N-(3-dimethylaminopropyl)-N′-ethylcarbodiimide hydrochloride (575 mg, 3.0 mmol, 1.5 eq.) and 4-dimethylaminopyridine (367 mg, 3.0 mmol, 1.5 eq.) were added in sequence. The mixture was stirred at r.t. for 18 hours. The solution was diluted with AcOEt (200 mL). The diluted solution was washed with 1N aq. HCl (3×100 mL), sat. aq. NaHCO₃ soln. (3×100 mL), sat. aq. NaCl soln. (1×100 mL), dried over MgSO₄, and concentrated in vacuo. The residue was purified by CC (DCM/AcOEt 7:3 to 6:4) to afford the desired amide as a white solid.

LC-MS (A): t_{R}=3.20 min; [M+H]⁺=450.30.

### Example 2

### 2-[1-(2,3-Dimethyl-phenyl)-1H-tetrazol-5-ylsulfanyl]-N-[2-(4-methoxy-3-methyl-benzyl)-2H-pyrazol-3-yl]-acetamide

### Step 1: 2-(4-Methoxy-3-methyl-benzyl)-2H-pyrazol-3-ylamine

Following the procedure described in Example 1, Step 4, but using the corresponding aldehyde, the desired pyrazole was obtained as a brown oil.

LC-MS (A): t_{R}=2.04 min; [M+H]⁺=218.3.

### Step 2

To a solution of [1-(2,3-dimethyl-phenyl)-1H-tetrazol-5-ylsulfanyl]-acetic acid (50 mg, 0.19 mmol, 1.0 eq.) and 2-(4-Methoxy-3-methyl-benzyl)-2H-pyrazol-3-ylamine (45 mg, 0.21 mmol, 1.1 eq.) in DMF (1.2 mL), N-(3-dimethylaminopropyl)-N′-ethylcarbodiimide hydrochloride (54 mg, 0.28 mmol, 1.5 eq.) and 4-dimethylaminopyridine (35 mg, 0.28 mmol, 1.5 eq.) were added in sequence. The mixture was stirred at r.t. for 18 hours. The mixture was purified by prep. HPLC and evaporated to afford the desired title compound.

LC-MS (B): t_{R}=0.91 min; [M+H]⁺=464.17.

### Example 3

### 2-[1-(2,3-Dimethyl-phenyl)-1H-tetrazol-5-ylsulfanyl]-N-[2-(3-fluoro-4-methoxy-benzyl)-2H-pyrazol-3-yl]-acetamide

### Step 1: 2-(3-Fluoro-4-methoxy-benzyl)-2H-pyrazol-3-ylamine

Following the procedure described in Example 1, Step 4, but using the corresponding aldehyde, the desired pyrazole was obtained as a brown oil.

LC-MS (A): t_{R}=1.55 min; [M+H]⁺=222.0.

### Step 2

The title compound was obtained following the procedure described in Example 2, Step 2.

LC-MS (B): t_{R}=0.88 min; [M+H]⁺=468.16

### Example 4

### N-(2-Benzyl-2H-pyrazol-3-yl)-2-[1-(2,3-dimethyl-phenyl)-1H-tetrazol-5-ylsulfanyl]-acetamide

### Step 1: 2-Benzyl-2H-pyrazol-3-ylamine

Following the procedure described in Example 1, Step 4, but using the corresponding aldehyde, the desired pyrazole was obtained as an orange oil.

LC-MS (A): t_{R}=1.09 min; [M+H]⁺=174.10.

### Step 2

The title compound was obtained following the procedure described in Example 2, Step 2.

LC-MS (B): t_{R}=0.88 min; [M+H]⁺=420.14.

### Example 5

### 2-[1-(2,3-Dimethyl-phenyl)-1H-tetrazol-5-ylsulfanyl]-N-[2-(4-isopropyl-benzyl)-2H-pyrazol-3-yl]-acetamide

### Step 1: 2-(4-Isopropyl-benzyl)-2H-pyrazol-3-ylamine

Following the procedure described in Example 1, Step 4, but using the corresponding aldehyde, the desired pyrazole was obtained as a yellow solid.

LC-MS (A): t_{R}=2.54 min; [M+H]⁺=216.30.

### Step 2

The title compound was obtained following the procedure described in Example 2, Step 2.

LC-MS (B): t_{R}=0.97 min; [M+H]⁺=462.20.

### Example 6

### 2-[1-(2,3-Dimethyl-phenyl)-1H-tetrazol-5-ylsulfanyl]-N-(2-naphthalen-2-ylmethyl-2H-pyrazol-3-yl)-acetamide

### Step 1: 2-Naphthalen-2-ylmethyl-2H-pyrazol-3-ylamine

Following the procedure described in Example 1, Step 4, but using the corresponding aldehyde, the desired pyrazole was obtained as a pale yellow solid.

LC-MS (A): t_{R}=2.40 min; [M+H]⁺=224.10.

### Step 2:

The title compound was obtained following the procedure described in Example 2, Step 2.

LC-MS (B): t_{R}=0.94 min; [M+H]⁺=470.17.

### Example 7

### 2-[1-(2,3-Dimethyl-phenyl)-1H-tetrazol-5-ylsulfanyl]-N-[2-(4-methyl-benzyl)-2H-pyrazol-3-yl]-acetamide

### Step 1: 2-(4-Methyl-benzyl)-2H-pyrazol-3-ylamine

Following the procedure described in Example 1, Step 4, but using the corresponding aldehyde, the desired pyrazole was obtained as a brown oil.

LC-MS (A): t_{R}=1.86 min; [M+H]⁺=188.3.

### Step 2

The title compound was obtained following the procedure described in Example 2, Step 2.

LC-MS (B): t_{R}=0.91 min; [M+H]⁺=434.14.

### Example 8

### 2-[1-(2,3-Dimethyl-phenyl)-1H-tetrazol-5-ylsulfanyl]-N-[2-(3-methoxy-benzyl)-2H-pyrazol-3-yl]-acetamide

### Step 1: 2-(3-Methoxy-benzyl)-2H-pyrazol-3-ylamine

Following the procedure described in Example 1, Step 4, but using the corresponding aldehyde, the desired pyrazole was obtained as a yellow oil.

LC-MS (A): t_{R}=1.49 min; [M+H]⁺=204.2.

### Step 2

The title compound was obtained following the procedure described in Example 2, Step 2.

LC-MS (B): t_{R}=0.88 min; [M+H]⁺=450.15.

### Example 9

### 2-[1-(2,3-Dimethyl-phenyl)-1H-tetrazol-5-ylsulfanyl]-N-[2-(2-methoxy-benzyl)-2H-pyrazol-3-yl]-acetamide

### Step 1: 2-(2-Methoxy-benzyl)-2H-pyrazol-3-ylamine

Following the procedure described in Example 1, Step 4, but using the corresponding aldehyde, the desired pyrazole was obtained as a brown oil.

LC-MS (A): t_{R}=1.33 min; [M+H]⁺=204.3.

### Step 2

The title compound was obtained following the procedure described in Example 2, Step 2.

LC-MS (B): t_{R}=0.90 min; [M+H]⁺=450.13

### Example 10

### N-[2-(3,4-Dimethoxy-benzyl)-2H-pyrazol-3-yl]-2-[1-(2,3-dimethyl-phenyl)-1H-tetrazol-5-ylsulfanyl]-acetamide

### Step 1: 2-(3,4-Dimethoxy-benzyl)-2H-pyrazol-3-ylamine

Following the procedure described in Example 1, Step 4, but using the corresponding aldehyde, the desired pyrazole was obtained as a yellow oil.

LC-MS (A): t_{R}=1.02 min; [M+H]⁺=234.30.

### Step 2

The title compound was obtained following the procedure described in Example 2, Step 2.

LC-MS (B): t_{R}=0.85 min; [M+H]⁺=480.23.

### Example 11

### N-[2-(2,4-Dimethoxy-benzyl)-2H-pyrazol-3-yl]-2-[1-(2,3-dimethyl-phenyl)-1H-tetrazol-5-ylsulfanyl]-acetamide

### Step 1: 2-(2,4-Dimethoxy-benzyl)-2H-pyrazol-3-ylamine

Following the procedure described in Example 1, Step 4, but using the corresponding aldehyde, the desired pyrazole was obtained as an orange oil.

LC-MS (A): t_{R}=1.63 min; [M+H]⁺=234.2.

### Step 2

The title compound was obtained following the procedure described in Example 2, Step 2.

LC-MS (B): t_{R}=0.90 min; [M+H]⁺=480.17.

### Example 12

### N-[2-(4-n-Butoxy-benzyl)-2H-pyrazol-3-yl]-2-[1-(2,3-dimethyl-phenyl)-1H-tetrazol-5-ylsulfanyl]-acetamide

### Step 1: 2-(4-n-Butoxy-benzyl)-2H-pyrazol-3-ylamine

Following the procedure described in Example 1, Step 4, but using the corresponding aldehyde, the desired pyrazole was obtained as a yellow solid.

LC-MS (A): t_{R}=2.68 min; [M+H]⁺=246.30.

### Step 2

The title compound was obtained following the procedure described in Example 2, Step 2.

LC-MS (B): t_{R}=0.98 min; [M+H]⁺=492.19.

### Example 13

### 2-[1-(2,3-Dimethyl-phenyl)-1H-tetrazol-5-ylsulfanyl]-N-[2-(4-trifluoromethoxy-benzyl)-2H-pyrazol-3-yl]-acetamide

### Step 1: 2-(4-Trifluoromethoxy-benzyl)-2H-pyrazol-3-ylamine

Following the procedure described in Example 1, Step 4, but using the corresponding aldehyde, the desired pyrazole was obtained as a brown oil.

LC-MS (A): t_{R}=2.64 min; [M+H]⁺=258.2.

### Step 2

The title compound was obtained following the procedure described in Example 2, Step 2.

LC-MS (B): t_{R}=0.95 min; [M+H]⁺=504.12.

### Example 14

### 2-[1-(2,3-Dimethyl-phenyl)-1H-tetrazol-5-ylsulfanyl]-N-[2-(4-methoxy-2,5-dimethyl-benzyl)-2H-pyrazol-3-yl]-acetamide

### Step 1: 2-(4-Methoxy-2,5-dimethyl-benzyl)-2H-pyrazol-3-ylamine

Following the procedure described in Example 1, Step 4, but using the corresponding aldehyde, the desired pyrazole was obtained as a beige solid.

LC-MS (A): t_{R}=2.26 min; [M+H]⁺=232.20.

### Step 2

The title compound was obtained following the procedure described in Example 2, Step 2.

LC-MS (B): t_{R}=0.93 min; [M+H]⁺=478.19.

### Example 15

### 2-[1-(2,3-Dimethyl-phenyl)-1H-tetrazol-5-ylsulfanyl]-N-[2-(4-trifluoromethyl-benzyl)-2H-pyrazol-3-yl]-acetamide

### Step 1: 2-(4-Trifluoromethyl-benzyl)-2H-pyrazol-3-ylamine

Following the procedure described in Example 1, Step 4, but using the corresponding aldehyde, the desired pyrazole was obtained as a brown solid.

LC-MS (A): t_{R}=2.52 min; [M+H]⁺=242.10.

### Step 2

The title compound was obtained following the procedure described in Example 2, Step 2.

LC-MS (B): t_{R}=0.93 min; [M+H]⁺=488.16.

### Example 16

### 2-[1-(2,3-Dimethyl-phenyl)-1H-tetrazol-5-ylsulfanyl]-N-[2-(4-fluoro-benzyl)-2H-pyrazol-3-yl]-acetamide

### Step 1: 2-(4-Fluoro-benzyl)-2H-pyrazol-3-ylamine

Following the procedure described in Example 1, Step 4, but using the corresponding aldehyde, the desired pyrazole was obtained as an orange oil.

LC-MS (A): t_{R}=1.44 min; [M+H]⁺=192.0.

### Step 2

The title compound was obtained following the procedure described in Example 2, Step 2.

LC-MS (B): t_{R}=0.89 min; [M+H]⁺=438.14.

### Example 17

### 2-[1-(2,3-Dimethyl-phenyl)-1H-tetrazol-5-ylsulfanyl]-N-[2-(3-fluoro-benzyl)-2H-pyrazol-3-yl]-acetamide

### Step 1: 2-(3-Fluoro-benzyl)-2H-pyrazol-3-ylamine

Following the procedure described in Example 1, Step 4, but using the corresponding aldehyde, the desired pyrazole was obtained as an orange oil.

LC-MS (A): t_{R}=1.60 min; [M+H]⁺=192.2.

### Step 2

The title compound was obtained following the procedure described in Example 2, Step 2.

LC-MS (B): t_{R}=0.89 min; [M+H]⁺=438.13.

### Example 18

### 2-[1-(2,3-Dimethyl-phenyl)-1H-tetrazol-5-ylsulfanyl]-N-[2-(2-fluoro-benzyl)-2H-pyrazol-3-yl]-acetamide

### Step 1: 2-(2-Fluoro-benzyl)-2H-pyrazol-3-ylamine

Following the procedure described in Example 1, Step 4, but using the corresponding aldehyde, the desired pyrazole was obtained as an orange oil.

LC-MS (A): t_{R}=1.38 min; [M+H]⁺=192.20.

### Step 2

The title compound was obtained following the procedure described in Example 2, Step 2.

LC-MS (B): t_{R}=0.88 min; [M+H]⁺=438.13.

### Example 19

### N-(2-Benzo[1,3]dioxol-5-ylmethyl-2H-pyrazol-3-yl)-2-[1-(2,3-dimethyl-phenyl)-1H-tetrazol-5-ylsulfanyl]-acetamide

### Step 1: 2-Benzo[1,3]-dioxol-5-ylmethyl-2H-pyrazol-3-ylamine

Following the procedure described in Example 1, Step 4, but using the corresponding aldehyde, the desired pyrazole was obtained as a yellow solid.

LC-MS (A): t_{R}=1.21 min; [M+H]⁺=218.20.

### Step 2

The title compound was obtained following the procedure described in Example 2, Step 2.

LC-MS (B): t_{R}=0.87 min; [M+H]⁺=464.13.

### Example 20

### 2-[1-(2,3-Dimethyl-phenyl)-1H-tetrazol-5-ylsulfanyl]-N-[2-(4-methoxy-2,3-dimethyl-benzyl)-2H-pyrazol-3-yl]-acetamide

### Step 1: 2-(4-Methoxy-2,3-dimethyl-benzyl)-2H-pyrazol-3-ylamine

Following the procedure described in Example 1, Step 4, but using the corresponding aldehyde, the desired pyrazole was obtained as a brown solid.

LC-MS (A): t_{R}=2.28 min; [M+H]⁺=232.30.

### Step 2

The title compound was obtained following the procedure described in Example 2, Step 2.

LC-MS (B): t_{R}=0.93 min; [M+H]⁺=478.22.

### Example 21

### 2-[1-(2,3-Dimethyl-phenyl)-1H-tetrazol-5-ylsulfanyl]-N-[2-(4-ethoxy-benzyl)-2H-pyrazol-3-yl]-acetamide

### Step 1: 2-(4-Ethoxy-benzyl)-2H-pyrazol-3-ylamine

Following the procedure described in Example 1, Step 4, but using the corresponding aldehyde, the desired pyrazole was obtained as a yellow solid.

LC-MS (A): t_{R}=1.98 min; [M+H]⁺=218.3.

### Step 2

The title compound was obtained following the procedure described in Example 2, Step 2.

LC-MS (B): t_{R}=0.91 min; [M+H]⁺=464.20.

### Example 22

### 2-[1-(2,3-Dimethyl-phenyl)-1H-tetrazol-5-ylsulfanyl]-N-[2-(2-methyl-benzyl)-2H-pyrazol-3-yl]-acetamide

### Step 1: 2-(2-Methyl-benzyl)-2H-pyrazol-3-ylamine

Following the procedure described in Example 1, Step 4, but using the corresponding aldehyde, the desired pyrazole was obtained as a brown oil.

LC-MS (A): t_{R}=1.71 min; [M+H]⁺=188.3.

### Step 2

The title compound was obtained following the procedure described in Example 2, Step 2.

LC-MS (B): t_{R}=0.90 min; [M+H]⁺=434.13.

### Example 23

### 2-[1-(2,3-Dimethyl-phenyl)-1H-tetrazol-5-ylsulfanyl]-N-[2-(3-methyl-benzyl)-2H-pyrazol-3-yl]-acetamide

### Step 1: 2-(3-Methyl-benzyl)-2H-pyrazol-3-ylamine

Following the procedure described in Example 1, Step 4, but using the corresponding aldehyde, the desired pyrazole was obtained as a brown oil.

LC-MS (B): t_{R}=0.66 min; [M+H]⁺=188.52.

### Step 2

The title compound was obtained following the procedure described in Example 2, Step 2.

LC-MS (B): t_{R}=0.91 min; [M+H]⁺=434.13.

### Example 24

### 2-[1-(2,3-Dimethyl-phenyl)-1H-tetrazol-5-ylsulfanyl]-N-[2-(3,4,5-trimethoxy-benzyl)-2H-pyrazol-3-yl]-acetamide

### Step 1: 2-(3,4,5-Trimethoxy-benzyl)-2H-pyrazol-3-ylamine

Following the procedure described in Example 1, Step 4, but using the corresponding aldehyde, the desired pyrazole was obtained as an orange oil.

LC-MS (A): t_{R}=1.50 min; [M+H]⁺=264.10.

### Step 2

The title compound was obtained following the procedure described in Example 2, Step 2.

LC-MS (B): t_{R}=0.85 min; [M+H]⁺=510.16.

### Example 25

### 2-[1-(2,3-Dimethyl-phenyl)-1H-tetrazol-5-ylsulfanyl]-N-[2-(2,3,4-trimethoxy-benzyl)-2H-pyrazol-3-yl]-acetamide

### Step 1: 2-(2,3,4-Trimethoxy-benzyl)-2H-pyrazol-3-ylamine

Following the procedure described in Example 1, Step 4, but using the corresponding aldehyde, the desired pyrazole was obtained as an orange solid.

LC-MS (A): t_{R}=1.73 min; [M+H]⁺=264.20.

### Step 2

The title compound was obtained following the procedure described in Example 2, Step 2.

LC-MS (B): t_{R}=0.89 min; [M+H]⁺=510.18.

### Example 26

### 2-[1-(2,3-Dimethyl-phenyl)-1H-tetrazol-5-ylsulfanyl]-N-[2-(2-trifluoromethyl-benzyl)-2H-pyrazol-3-yl]-acetamide

### Step 1: 2-(2-Trifluoromethyl-benzyl)-2H-pyrazol-3-ylamine

Following the procedure described in Example 1, Step 4, but using the corresponding aldehyde, the desired pyrazole was obtained as an orange solid.

LC-MS (B): t_{R}=0.69 min; [M+H]⁺=241.96.

### Step 2

The title compound was obtained following the procedure described in Example 2, Step 2.

LC-MS (B): t_{R}=0.92 min; [M+H]⁺=488.12.

### Example 27

### 2-[1-(2,3-Dimethyl-phenyl)-1H-tetrazol-5-ylsulfanyl]-N-[2-(2,4,5-trimethoxy-benzyl)-2H-pyrazol-3-yl]-acetamide

### Step 1: 2-(2,4,5-Trimethoxy-benzyl)-2H-pyrazol-3-ylamine

Following the procedure described in Example 1, Step 4, but using the corresponding aldehyde, the desired pyrazole was obtained as a yellow solid.

LC-MS (A): t_{R}=1.39 min; [M+H]⁺=264.30.

### Step 2

The title compound was obtained following the procedure described in Example 2, Step 2.

LC-MS (B): t_{R}=0.87 min; [M+H]⁺=510.17.

### Example 28

### N-[2-(3-Chloro-4-methyl-benzyl)-2H-pyrazol-3-yl]-2-[1-(2,3-dimethyl-phenyl)-1H-tetrazol-5-ylsulfanyl]-acetamide

### Step 1: 2-(3-Chloro-4-methyl-benzyl)-2H-pyrazol-3-ylamine

Following the procedure described in Example 1, Step 4, but using the corresponding aldehyde, the desired pyrazole was obtained as a brown solid.

LC-MS (A): t_{R}=2.47 min; [M+H]⁺=222.10.

### Step 2

The title compound was obtained following the procedure described in Example 2, Step 2.

LC-MS (B): t_{R}=0.94 min; [M+H]⁺=468.12.

### Example 29

### 2-[1-(2,3-Dimethyl-phenyl)-1H-tetrazol-5-ylsulfanyl]-N-(2-pyridin-2-ylmethyl-2H-pyrazol-3-yl)-acetamide

### Step 1: 2-Pyridin-2-ylmethyl-2H-pyrazol-3-ylamine

Following the procedure described in Example 1, Step 4, but using the corresponding aldehyde, the desired pyrazole was obtained as a black solid.

LC-MS (A): t_{R}=0.62 min; [M+H]⁺=175.1.

### Step 2

The title compound was obtained following the procedure described in Example 2, Step 2.

LC-MS (B): t_{R}=0.79 min; [M+H]⁺=421.11.

### Example 30

### N-[2-(4-tert-Butyl-benzyl)-2H-pyrazol-3-yl]-2-[1-(2,3-dimethyl-phenyl)-1H-tetrazol-5-ylsulfanyl]-acetamide

### Step 1: 2-(4-tent-Butyl-benzyl)-2H-pyrazol-3-ylamine

Following the procedure described in Example 1, Step 4, but using the corresponding aldehyde, the desired pyrazole was obtained as a yellow solid.

LC-MS (A): t_{R}=2.72 min; [M+H]⁺=230.10.

### Step 2

The title compound was obtained following the procedure described in Example 2, Step 2.

LC-MS (B): t_{R}=0.98 min; [M+H]⁺=476.21.

### Example 31

### 2-[1-(2,3-Dimethyl-phenyl)-1H-tetrazol-5-ylsulfanyl]-N-[2-(6-methoxy-pyridin-3-ylmethyl)-2H-pyrazol-3-yl]-acetamide

### Step 1: 2-(6-Methoxy-pyridin-3-ylmethyl)-2H-pyrazol-3-ylamine

Following the procedure described in Example 1, Step 4, but using the corresponding aldehyde, the desired pyrazole was obtained as an orange solid.

LC-MS (A): t_{R}=0.94 min; [M+H]⁺=205.20.

### Step 2

The title compound was obtained following the procedure described in Example 2, Step 2.

LC-MS (B): t_{R}=0.81 min; [M+H]⁺=451.11

### Example 32

### 2-[1-(2,3-Dimethyl-phenyl)-1H-tetrazol-5-ylsulfanyl]-N-(2-pyridin-4-ylmethyl-2H-pyrazol-3-yl)-acetamide

### Step 1: 2-Pyridin-4-ylmethyl-2H-pyrazol-3-ylamine

Following the procedure described in Example 1, Step 4, but using the corresponding aldehyde, the desired pyrazole was obtained as an orange solid.

LC-MS (A): t_{R}=0.57 min; [M+H]⁺=175.10.

### Step 2: 2-[1-(2,3-Dimethyl-phenyl)-1H-tetrazol-5-ylsulfanyl]-N-(2-pyridin-4-ylmethyl-2H-pyrazol-3-yl)-acetamide

The title compound was obtained following the procedure described in Example 2, Step 2.

LC-MS (B): t_{R}=0.72 min; [M+H]⁺=421.09.

### Example 33

### 2-[1-(2,3-Dimethyl-phenyl)-1H-tetrazol-5-ylsulfanyl]-N-[2-(1H-indol-6-ylmethyl)-2H-pyrazol-3-yl]-acetamide

### Step 1: 2-(1H-Indol-6-ylmethyl)-2H-pyrazol-3-ylamine

Following the procedure described in Example 1, Step 4, but using the corresponding aldehyde, the desired pyrazole was obtained as an orange foam.

LC-MS (A): t_{R}=0.98 min; [M+H]⁺=213.20.

### Step 2

The title compound was obtained following the procedure described in Example 2, Step 2.

LC-MS (B): t_{R}=0.85 min; [M+H]⁺=459.03.

### Example 34

### N-[2-(4-Dimethylamino-benzyl)-2H-pyrazol-3-yl]-2-[1-(2,3-dimethyl-phenyl)-1H-tetrazol-5-ylsulfanyl]-acetamide

### Step 1: 2-(4-Dimethylamino-benzyl)-2H-pyrazol-3-ylamine

Following the procedure described in Example 1, Step 4, but using the corresponding aldehyde, the desired pyrazole was obtained as an orange solid.

LC-MS (A): t_{R}=0.63 min; [M+H]⁺=217.3.

### Step 2

The title compound was obtained following the procedure described in Example 2, Step 2.

LC-MS (B): t_{R}=0.89 min; [M+H]⁺=463.20.

### Example 35

### 2-[1-(2,3-Dimethyl-phenyl)-1H-tetrazol-5-ylsulfanyl]-N-(2-thiophen-3-ylmethyl-2H-pyrazol-3-yl)-acetamide

### Step 1: 2-Thiophen-3-ylmethyl-2H-pyrazol-3-ylamine

Following the procedure described in Example 1, Step 4, but using the corresponding aldehyde, the desired pyrazole was obtained as a brown oil.

LC-MS (A): t_{R}=0.90 min; [M+H]⁺=180.1.

### Step 2

The title compound was obtained following the procedure described in Example 2, Step 2.

LC-MS (B): t_{R}=0.85 min; [M+H]⁺=425.88.

### Example 36

### 2-[1-(2,3-Dimethyl-phenyl)-1H-tetrazol-5-ylsulfanyl]-N-(2-thiophen-2-ylmethyl-2H-pyrazol-3-yl)-acetamide

### Step 1: 2-Thiophen-2-ylmethyl-2H-pyrazol-3-ylamine

Following the procedure described in Example 1, Step 4, but using the corresponding aldehyde, the desired pyrazole was obtained as a brown oil.

LC-MS (A): t_{R}=1.00 min; [M+H]⁺=180.3.

### Step 2

The title compound was obtained following the procedure described in Example 2, Step 2.

LC-MS (B): t_{R}=0.84 min; [M+H]⁺=425.82

### Example 37

### 2-[1-(2,3-Dimethyl-phenyl)-1H-tetrazol-5-ylsulfanyl]-N-(2-pyridin-3-ylmethyl-2H-pyrazol-3-yl)-acetamide

### Step 1: 2-Pyridin-3-ylmethyl-2H-pyrazol-3-ylamine

Following the procedure described in Example 1, Step 4, but using the corresponding aldehyde, the desired pyrazole was obtained as an orange solid.

LC-MS (A): t_{R}=0.59 min; [M+H]⁺=175.1.

### Step 2

The title compound was obtained following the procedure described in Example 2, Step 2.

LC-MS (B): t_{R}=0.74 min; [M+H]⁺=421.09.

### Example 38

### N-[2-(4,5-Dimethyl-furan-2-ylmethyl)-2H-pyrazol-3-yl]-2-[1-(2,3-dimethyl-phenyl)-1H-tetrazol-5-ylsulfanyl]-acetamide

### Step 1: 2-(4,5-Dimethyl-furan-2-ylmethyl)-2H-pyrazol-3-ylamine

Following the procedure described in Example 1, Step 4, but using the corresponding aldehyde, the desired pyrazole was obtained as a brown solid.

LC-MS (A): t_{R}=1.88 min; [M+H]⁺=192.10.

### Step 2

The title compound was obtained following the procedure described in Example 2, Step 2.

LC-MS (B): t_{R}=0.89 min; [M+H]⁺=438.12.

### Example 39

### 2-[1-(2,3-Dimethyl-phenyl)-1H-tetrazol-5-ylsulfanyl]-N-[2-(4-isopropoxy-benzyl)-2H-pyrazol-3-yl]-acetamide

### Step 1: 2-(4-Isopropoxy-benzyl)-2H-pyrazol-3-ylamine

Following the procedure described in Example 1, Step 4, but using the corresponding aldehyde, the desired pyrazole was obtained as a brown oil.

LC-MS (A): t_{R}=2.29 min; [M+H]⁺=232.3.

### Step 2

The title compound was obtained following the procedure described in Example 2, Step 2.

LC-MS (B): t_{R}=0.92 min; [M+H]⁺=478.18.

### Example 40

### 2-[1-(2,3-Dimethyl-phenyl)-1H-tetrazol-5-ylsulfanyl]-N-[2-(4-propoxy-benzyl)-2H-pyrazol-3-yl]-acetamide

### Step 1: 2-(4-Propoxy-benzyl)-2H-pyrazol-3-ylamine

Following the procedure described in Example 1, Step 4, but using the corresponding aldehyde, the desired pyrazole was obtained as a brown oil.

LC-MS (A): t_{R}=2.42 min; [M+H]⁺=232.3.

### Step 2

The title compound was obtained following the procedure described in Example 2, Step 2.

LC-MS (B): t_{R}=0.94 min; [M+H]⁺=478.19.

### Example 41

### N-[2-(2-Chloro-benzyl)-2H-pyrazol-3-yl]-2-[1-(2,3-dimethyl-phenyl)-1H-tetrazol-5-ylsulfanyl]-acetamide

### Step 1: 2-(2-Chloro-benzyl)-2H-pyrazol-3-ylamine

Following the procedure described in Example 1, Step 4, but using the corresponding aldehyde, the desired pyrazole was obtained as an orange solid.

LC-MS (A): t_{R}=1.96 min; [M+H]⁺=208.2.

### Step 2

The title compound was obtained following the procedure described in Example 2, Step 2.

LC-MS (B): t_{R}=0.89 min; [M+H]⁺=454.08.

### Example 42

### N-[2-(3-Chloro-benzyl)-2H-pyrazol-3-yl]-2-[1-(2,3-dimethyl-phenyl)-1H-tetrazol-5-ylsulfanyl]-acetamide

### Step 1: 2-(3-Chloro-benzyl)-2H-pyrazol-3-ylamine

Following the procedure described in Example 1, Step 4, but using the corresponding aldehyde, the desired pyrazole was obtained as a brown oil.

LC-MS (A): t_{R}=2.19 min; [M+H]⁺=208.0.

### Step 2

The title compound was obtained following the procedure described in Example 2, Step 2.

LC-MS (B): t_{R}=0.90 min; [M+H]⁺=454.07.

### Example 43

### N-[2-(3,4-Dichloro-benzyl)-2H-pyrazol-3-yl]-2-[1-(2,3-dimethyl-phenyl)-1H-tetrazol-5-ylsulfanyl]-acetamide

### Step 1: 2-(3,4-Dichloro-benzyl)-2H-pyrazol-3-ylamine

Following the procedure described in Example 1, Step 4, but using the corresponding aldehyde, the desired pyrazole was obtained as an orange oil.

LC-MS (A): t_{R}=2.63 min; [M+H]⁺=242.0.

### Step 2

The title compound was obtained following the procedure described in Example 2, Step 2.

LC-MS (B): t_{R}=0.93 min; [M+H]⁺=488.04.

### Example 44

### N-[2-(4-Chloro-benzyl)-2H-pyrazol-3-yl]-2-[1-(2,3-dimethyl-phenyl)-1H-tetrazol-5-ylsulfanyl]-acetamide

### Step 1: 2-(4-Chloro-benzyl)-2H-pyrazol-3-ylamine

Following the procedure described in Example 1, Step 4, but using the corresponding aldehyde, the desired pyrazole was obtained as a brown solid.

LC-MS (A): t_{R}=2.21 min; [M+H]⁺=208.2.

### Step 2

The title compound was obtained following the procedure described in Example 2, Step 2.

LC-MS (B): t_{R}=0.91 min; [M+H]⁺=454.07.

### Example 45

### N-[2-(4-Methoxy-benzyl)-2H-pyrazol-3-yl]-2-(1-o-tolyl-1H-tetrazol-5-ylsulfanyl)-acetamide

### Step 1: (1-o-Tolyl-1H-tetrazol-5-ylsulfanyl)-acetic acid

Following the procedure described in Example 1, Steps 1 to 3, but using the corresponding isothiocyanate, the desired acid was obtained as a white solid.

LC-MS (A): t_{R}=2.66 min; [M+H]⁺=250.9.

### Step 2

To a solution of (1-o-Tolyl-1H-tetrazol-5-ylsulfanyl)-acetic acid (49 mg, 0.20 mmol, 1.0 eq.) and 2-(4-Methoxy-benzyl)-2H-pyrazol-3-ylamine (40 mg, 0.20 mmol, 1.0 eq.) in DMF (1.2 mL), N-(3-dimethylaminopropyl)-N′-ethylcarbodiimide hydrochloride (57 mg, 0.30 mmol, 1.5 eq.) and 4-dimethylaminopyridine (36 mg, 0.30 mmol, 1.5 eq.) were added in sequence. The mixture was stirred at r.t. for 18 hours. The mixture was purified by prep. HPLC and evaporated (speedvac) to afford the desired amide.

LC-MS (B): t_{R}=0.99 min; [M+H]⁺=436.32.

### Example 46

### 2-[1-(2,5-Dimethyl-phenyl)-1H-tetrazol-5-ylsulfanyl]-N-[2-(4-methoxy-benzyl)-2H-pyrazol-3-yl]-acetamide

### Step 1: [1-(2,5-Dimethyl-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.86 min; [M+H]⁺=265.3.

### Step 2

The title compound was obtained following the procedure described in Example 45, Step 2.

LC-MS (B): t_{R}=1.02 min; [M+H]⁺=450.43.

### Example 47

### 2-[1-(2,4-Dimethyl-phenyl)-1H-tetrazol-5-ylsulfanyl]-N-[2-(4-methoxy-benzyl)-2H-pyrazol-3-yl]-acetamide

### Step 1: [1-(2,4-Dimethyl-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.85 min; [M+H]⁺=265.3.

### Step 2

The title compound was obtained following the procedure described in Example 45, Step 2.

LC-MS (B): t_{R}=1.02 min; [M+H]⁺=450.38.

### Example 48

### 2-[1-(2,5-Dimethoxy-phenyl)-1H-tetrazol-5-ylsulfanyl]-N-[2-(4-methoxy-benzyl)-2H-pyrazol-3-yl]-acetamide

### Step 1: [1-(2,5-Dimethoxy-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.64 min; [M+H]⁺=297.4.

### Step 2

The title compound was obtained following the procedure described in Example 45, Step 2.

LC-MS (B): t_{R}=0.99 min; [M+H]⁺=482.37.

### Example 49

### N-[2-(4-Methoxy-benzyl)-2H-pyrazol-3-yl]-2-(1-phenyl-1H-tetrazol-5-ylsulfanyl)-acetamide

### Step 1: (1-Phenyl-1H-tetrazol-5-ylsulfanyl)-acetic acid

Following the procedure described in Example 1, Steps 1 to 3, but using the corresponding isothiocyanate, the desired acid was obtained as a white solid.

LC-MS (A): t_{R}=2.43 min; [M+H]⁺=237.0.

### Step 2

The title compound was obtained following the procedure described in Example 45, Step 2.

LC-MS (B): t_{R}=0.97 min; [M+H]⁺=422.54.

### Example 50

### 2-[1-(2,6-Dimethyl-phenyl)-1H-tetrazol-5-ylsulfanyl]-N-[2-(4-methoxy-benzyl)-2H-pyrazol-3-yl]-acetamide

### Step 1: [1-(2,6-Dimethyl-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.81 min; [M+H]⁺=265.4.

### Step 2

The title compound was obtained following the procedure described in Example 45, Step 2.

LC-MS (B): t_{R}=0.86 min; [M+H]⁺=450.14.

### Example 51

### N-[2-(4-Methoxy-benzyl)-2H-pyrazol-3-yl]-2-[1-(2-methoxy-phenyl)-1H-tetrazol-5-ylsulfanyl]-acetamide

### Step 1: [1-(2-Methoxy-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.81 min; [M+H]⁺=265.4.

### Step 2

The title compound was obtained following the procedure described in Example 45, Step 2.

LC-MS (B): t_{R}=0.81 min; [M+H]⁺=452.12.

### Example 52

### N-[2-(4-Methoxy-benzyl)-2H-pyrazol-3-yl]-2-[1-(3-methoxy-phenyl)-1H-tetrazol-5-ylsulfanyl]-acetamide

### Step 1: [1-(3-Methoxy-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.60 min; [M+H]⁺=267.2.

### Step 2

The title compound was obtained following the procedure described in Example 45, Step 2.

LC-MS (B): t_{R}=0.82 min; [M+H]⁺=452.10.

### Example 53

### N-[2-(4-Methoxy-benzyl)-2H-pyrazol-3-yl]-2-(1-m-tolyl-1H-tetrazol-5-ylsulfanyl)-acetamide

### Step 1: (1-m-Tolyl-1H-tetrazol-5-ylsulfanyl)-acetic acid

Following the procedure described in Example 1, Steps 1 to 3, but using the corresponding isothiocyanate, the desired acid was obtained as a white solid.

LC-MS (A): t_{R}=2.69 min; [M+H]⁺=251.0.

### Step 2

The title compound was obtained following the procedure described in Example 45, Step 2.

LC-MS (B): t_{R}=0.84 min; [M+H]⁺=436.13.

### Example 54

### 2-[1-(2-Ethyl-6-methyl-phenyl)-1H-tetrazol-5-ylsulfanyl]-N-[2-(4-methoxy-benzyl)-2H-pyrazol-3-yl]-acetamide

### Step 1: [1-(2-Ethyl-6-methyl-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.94 min; [M+H]⁺=279.10.

### Step 2

The title compound was obtained following the procedure described in Example 45, Step 2.

LC-MS (B): t_{R}=0.89 min; [M+H]⁺=464.33.

### Example 55

### 2-[1-(2,4-Dimethoxy-phenyl)-1H-tetrazol-5-ylsulfanyl]-N-[2-(4-methoxy-benzyl)-2H-pyrazol-3-yl]-acetamide

### Step 1: [1-(2,4-Dimethoxy-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.64 min; [M+H]⁺=297.4

### Step 2

The title compound was obtained following the procedure described in Example 45, Step 2.

LC-MS (B): t_{R}=0.84 min; [M+H]⁺=482.15.

### Example 56

### N-[2-(4-Methoxy-benzyl)-2H-pyrazol-3-yl]-2-[1-(2-methoxy-5-methyl-phenyl)-1H-tetrazol-5-ylsulfanyl]-acetamide

### Step 1: [1-(2-Methoxy-5-methyl-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.73 min; [M+H]⁺=281.2.

### Step 2

The title compound was obtained following the procedure described in Example 45, Step 2.

LC-MS (B): t_{R}=0.84 min; [M+H]⁺=466.13.

### Example 57

### 2-[1-(2-Fluoro-phenyl)-1H-tetrazol-5-ylsulfanyl]-N-[2-(4-methoxy-benzyl)-2H-pyrazol-3-yl]-acetamide

### Step 1: [1-(2-Fluoro-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.52 min; [M+H]⁺=252.9.

### Step 2

The title compound was obtained following the procedure described in Example 45, Step 2.

LC-MS (B): t_{R}=0.80 min; [M+H]⁺=440.08.

### Example 58

### 2-[1-(2,5-Dimethyl-phenyl)-1H-tetrazol-5-ylsulfanyl]-N-[2-(3-fluoro-4-methoxy-benzyl)-2H-pyrazol-3-yl]-acetamide

The title compound was obtained following the procedure described in Example 2, Step 2, but using the corresponding pyrazole and the corresponding acid.

LC-MS (B): t_{R}=0.87 min; [M+H]⁺=468.14.

### Example 59

### 2-[1-(2,5-Dimethyl-phenyl)-1H-tetrazol-5-ylsulfanyl]-N-[2-(4-ethoxy-benzyl)-2H-pyrazol-3-yl]-acetamide

The title compound was obtained following the procedure described in Example 2, Step 2, but using the corresponding pyrazole and the corresponding acid.

LC-MS (B): t_{R}=0.90 min; [M+H]⁺=464.15.

### Example 60

### 2-[1-(2,4-Dimethoxy-phenyl)-1H-tetrazol-5-ylsulfanyl]-N-[2-(4-trifluoromethyl-benzyl)-2H-pyrazol-3-yl]-acetamide

The title compound was obtained following the procedure described in Example 2, Step 2, but using the corresponding pyrazole and the corresponding acid.

LC-MS (B): t_{R}=0.89 min; [M+H]⁺=520.14.

### Example 61

### 2-[1-(2,4-Dimethoxy-phenyl)-1H-tetrazol-5-ylsulfanyl]-N-[2-(4-fluoro-benzyl)-2H-pyrazol-3-yl]-acetamide

The title compound was obtained following the procedure described in Example 2, Step 2, but using the corresponding pyrazole and the corresponding acid.

LC-MS (B): t_{R}=0.85 min; [M+H]⁺=470.14.

### Example 62

### N-(2-Benzo[1,3]dioxol-5-ylmethyl-2H-pyrazol-3-yl)-2-[1-(2,4-dimethoxy-phenyl)-1H-tetrazol-5-ylsulfanyl]-acetamide

The title compound was obtained following the procedure described in Example 2, Step 2, but using the corresponding pyrazole and the corresponding acid.

LC-MS (B): t_{R}=0.84 min; [M+H]⁺=496.13.

### Example 63

### N-(2-Benzyl-2H-pyrazol-3-yl)-2-[1-(2,4-dimethoxy-phenyl)-1H-tetrazol-5-ylsulfanyl]-acetamide

The title compound was obtained following the procedure described in Example 2, Step 2, but using the corresponding pyrazole and the corresponding acid.

LC-MS (B): t_{R}=0.84 min; [M+H]⁺=452.16.

### Example 64

### 2-[1-(2,5-Dimethyl-phenyl)-1H-tetrazol-5-ylsulfanyl]-N-[2-(4-isopropyl-benzyl)-2H-pyrazol-3-yl]-acetamide

The title compound was obtained following the procedure described in Example 2, Step 2, but using the corresponding pyrazole and the corresponding acid.

LC-MS (B): t_{R}=0.96 min; [M+H]⁺=462.15.

### Example 65

### 2-[1-(2,5-Dimethyl-phenyl)-1H-tetrazol-5-ylsulfanyl]-N-[2-(4-methyl-benzyl)-2H-pyrazol-3-yl]-acetamide

The title compound was obtained following the procedure described in Example 2, Step 2, but using the corresponding pyrazole and the corresponding acid.

LC-MS (B): t_{R}=0.90 min; [M+H]⁺=434.12.

### Example 66

### 2-[1-(2,5-Dimethyl-phenyl)-1H-tetrazol-5-ylsulfanyl]-N-[2-(4-methoxy-3-methyl-benzyl)-2H-pyrazol-3-yl]-acetamide

The title compound was obtained following the procedure described in Example 2, Step 2, but using the corresponding pyrazole and the corresponding acid.

LC-MS (B): t_{R}=0.91 min; [M+H]⁺=464.18.

### Example 67

### 2-[1-(2,5-Dimethyl-phenyl)-1H-tetrazol-5-ylsulfanyl]-N-[2-(3-methyl-benzyl)-2H-pyrazol-3-yl]-acetamide

The title compound was obtained following the procedure described in Example 2, Step 2, but using the corresponding pyrazole and the corresponding acid.

LC-MS (B): t_{R}=0.90 min; [M+H]⁺=434.10.

### Example 68

### 2-[1-(2-Chloro-phenyl)-1H-tetrazol-5-ylsulfanyl]-N-[2-(4-methoxy-benzyl)-2H-pyrazol-3-yl]-acetamide

### Step 1: [1-(2-Chloro-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.65 min; [M+H]⁺=271.1.

### Step 2

The title compound was obtained following the procedure described in Example 45, Step 2.

LC-MS (B): t_{R}=0.83 min; [M+H]⁺=455.98.

### Example 69

### 2-[1-(2,5-Dichloro-phenyl)-1H-tetrazol-5-ylsulfanyl]-N-[2-(4-methoxy-benzyl)-2H-pyrazol-3-yl]-acetamide

### Step 1: [1-(2,5-Dichloro-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.86 min; [M+H]⁺=305.0.

### Step 2

The title compound was obtained following the procedure described in Example 45, Step 2.

LC-MS (B): t_{R}=0.87 min; [M+H]⁺=491.94.

### Example 70

### 2-[1-(3,5-Dimethyl-phenyl)-1H-tetrazol-5-ylsulfanyl]-N-[2-(4-methoxy-benzyl)-2H-pyrazol-3-yl]-acetamide

### Step 1: [1-(3,5-Dimethyl-phenyl)-1H-tetrazol-5-ylsulfanyl]-acetic acid

Following the procedure described in Example 1, Steps 1 to 3, but using the corresponding isothiocyanate, the desired acid was obtained as a white solid.

LC-MS (A): t_{R}=2.89 min; [M+H]⁺=265.2.

### Step 2

The title compound was obtained following the procedure described in Example 45, Step 2.

LC-MS (B): t_{R}=0.89 min; [M+H]⁺=450.06.

### Example 71

### 2-[1-(3-Chloro-phenyl)-1H-tetrazol-5-ylsulfanyl]-N-[2-(4-methoxy-benzyl)-2H-pyrazol-3-yl]-acetamide

### Step 1: [1-(3-Chloro-phenyl)-1H-tetrazol-5-ylsulfanyl]-acetic acid

Following the procedure described in Example 1, Steps 1 to 3, but using the corresponding isothiocyanate, the desired acid was obtained as a pale yellow solid.

LC-MS (A): t_{R}=2.75 min; [M+H]⁺=271.1.

### Step 2

The title compound was obtained following the procedure described in Example 45, Step 2.

LC-MS (B): t_{R}=0.86 min; [M+H]⁺=455.98.

### Example 72

### N-(2-Cyclohexylmethyl-2H-pyrazol-3-yl)-2-[1-(2,3-dimethyl-phenyl)-1H-tetrazol-5-ylsulfanyl]-acetamide

### Step 1: 2-Cyclohexylmethyl-2H-pyrazol-3-ylamine

Following the procedure described in Example 1, Step 4, but using the corresponding aldehyde, the desired pyrazole was obtained as an orange oil.

LC-MS (A): t_{R}=1.35 min; [M+H]⁺=180.3.

### Step 2

The title compound was obtained following the procedure described in Example 2, Step 2.

LC-MS (B): t_{R}=0.93 min; [M+H]⁺=425.76.

### Example 73

### 2-[1-(2,3-Dimethyl-phenyl)-1H-tetrazol-5-ylsulfanyl]-N-[2-(3-fluoro-pyridin-4-ylmethyl)-2H-pyrazol-3-yl]-acetamide

### Step 1: 2-(3-Fluoro-pyridin-4-ylmethyl)-2H-pyrazol-3-ylamine

Following the procedure described in Example 1, Step 4, but using the corresponding aldehyde, the desired pyrazole was obtained as an orange oil.

LC-MS (A): t_{R}=0.63 min; [M+H]⁺=193.3.

### Step 2

The title compound was obtained following the procedure described in Example 2, Step 2.

LC-MS (B): t_{R}=0.74 min; [M+H]⁺=439.02.

### Example 74

### 2-[1-(2,3-Dimethyl-phenyl)-1H-tetrazol-5-ylsulfanyl]-N-[2-(2-ethyl-butyl)-2H-pyrazol-3-yl]-acetamide

### Step 1: 2-(2-Ethyl-butyl)-2H-pyrazol-3-ylamine

Following the procedure described in Example 1, Step 4, but using the corresponding aldehyde, the desired pyrazole was obtained as an orange oil.

LC-MS (A): t_{R}=1.23 min; [M+H]⁺=168.2.

### Step 2

The title compound was obtained following the procedure described in Example 2, Step 2.

LC-MS (B): t_{R}=0.91 min; [M+H]⁺=414.11.

### Example 75

### 2-[1-(2,3-Dimethyl-phenyl)-1H-tetrazol-5-ylsulfanyl]-N-(2-phenethyl-2H-pyrazol-3-yl)-acetamide

### Step 1: 2-Phenethyl-2H-pyrazol-3-ylamine

Following the procedure described in Example 1, Step 4, but using the corresponding aldehyde, the desired pyrazole was obtained as a brown oil.

LC-MS (A): t_{R}=1.28 min; [M+H]⁺=188.3.

### Step 2

The title compound was obtained following the procedure described in Example 2, Step 2.

LC-MS (B): t_{R}=0.89 min; [M+H]⁺=434.09.

### Example 76

### N-(2-Cyclopropylmethyl-2H-pyrazol-3-yl)-2-[1-(2,3-dimethyl-phenyl)-1H-tetrazol-5-ylsulfanyl]-acetamide

### Step 1: 2-Cyclopropylmethyl-2H-pyrazol-3-ylamine

Following the procedure described in Example 1, Step 4, but using the corresponding aldehyde, the desired pyrazole was obtained as an orange oil.

LC-MS (A): t_{R}=0.62 min; [M+H]⁺=138.2.

### Step 2

The title compound was obtained following the procedure described in Example 2, Step 2.

LC-MS (B): t_{R}=0.83 min; [M+H]⁺=384.22.

### Example 77

### 2-[1-(2,3-Dimethyl-phenyl)-1H-tetrazol-5-ylsulfanyl]-N-[2-(2-methyl-butyl)-2H-pyrazol-3-yl]-acetamide

### Step 1: 2-(2-Methyl-butyl)-2H-pyrazol-3-ylamine

Following the procedure described in Example 1, Step 4, but using the corresponding aldehyde, the desired pyrazole was obtained as an orange oil.

LC-MS (A): t_{R}=0.86 min; [M+H]⁺=154.2.

### Step 2

The title compound was obtained following the procedure described in Example 2, Step 2.

LC-MS (B): t_{R}=0.89 min; [M+H]⁺=400.10.

### Example 78

### 2-[1-(2,3-Dimethyl-phenyl)-1H-tetrazol-5-ylsulfanyl]-N-[2-(5-methoxy-pyridin-3-ylmethyl)-2H-pyrazol-3-yl]-acetamide

### Step 1: 2-(5-Methoxy-pyridin-3-ylmethyl)-2H-pyrazol-3-ylamine

Following the procedure described in Example 1, Step 4, but using the corresponding aldehyde, the desired pyrazole was obtained as an orange oil.

LC-MS (A): t_{R}=0.63 min; [M+H]⁺=205.2.

### Step 2

The title compound was obtained following the procedure described in Example 2, Step 2.

LC-MS (B): t_{R}=0.74 min; [M+H]⁺=451.07.

### Example 79

### 2-[1-(2,3-Dimethyl-phenyl)-1H-tetrazol-5-ylsulfanyl]-N-[2-(3-phenyl-propyl)-2H-pyrazol-3-yl]-acetamide

### Step 1: 2-(3-Phenyl-propyl)-2H-pyrazol-3-ylamine

Following the procedure described in Example 1, Step 4, but using the corresponding aldehyde, the desired pyrazole was obtained as an yellow oil.

LC-MS (A): t_{R}=1.93 min; [M+H]⁺=202.20.

### Step 2

The title compound was obtained following the procedure described in Example 2, Step 2.

LC-MS (B): t_{R}=0.93 min; [M+H]⁺=448.02.

### Example 80

### 2-[1-(2,3-Dimethyl-phenyl)-1H-tetrazol-5-ylsulfanyl]-N-(2-ethyl-2H-pyrazol-3-yl)-acetamide

The title compound was obtained following the procedure described in Example 2, Step 2, starting from the commercially available 5-amino-1-ethylpyrazole.

LC-MS (B): t_{R}=0.82 min; [M+H]⁺=358.10.

### Example 81

### 2-[1-(2,3-Dimethyl-phenyl)-1H-tetrazol-5-ylsulfanyl]-N-[2-(4-phenoxy-benzyl)-2H-pyrazol-3-yl]-acetamide

### Step 1: 2-(4-Phenoxy-benzyl)-2H-pyrazol-3-ylamine

Following the procedure described in Example 1, Step 4, but using the corresponding aldehyde, the desired pyrazole was obtained as a brown solid.

LC-MS (A): t_{R}=2.69 min; [M+H]⁺=266.2.

### Step 2

The title compound was obtained following the procedure described in Example 2, Step 2.

LC-MS (B): t_{R}=0.96 min; [M+H]⁺=512.01.

### Example 82

### N-[2-(4-Benzyloxy-benzyl)-2H-pyrazol-3-yl]-2-[1-(2,3-dimethyl-phenyl)-1H-tetrazol-5-ylsulfanyl]-acetamide

### Step 1: 2-(4-Benzyloxy-benzyl)-2H-pyrazol-3-ylamine

Following the procedure described in Example 1, Step 4, but using the corresponding aldehyde, the desired pyrazole was obtained as a beige solid.

LC-MS (A2): t_{R}=0.68 min; [M+H]⁺=280.27.

### Step 2

The title compound was obtained following the procedure described in Example 2, Step 2.

LC-MS (A1): t_{R}=1.09 min; [M+H]⁺=526.17.

### Example 83

### 2-[1-(2,3-Dimethyl-phenyl)-1H-tetrazol-5-ylsulfanyl]-N-{2-[3-(4-trifluoromethyl-phenyl)-propyl]-2H-pyrazol-3-yl}-acetamide

### Step 1: 3-(4-Trifluoromethyl-phenyl)-propan-1-ol

To an ice-cooled homogeneous solution of 4-(trifluoromethyl)hydrocinnamic acid (20 g, 91.7 mmol 1 eq.) in anhydrous THF (500 ml), a solution of 1M BH₃ in THF (137.5 ml, 137.5 mmol, 1.5 eq.) was added dropwise over ca. 20 min. The resulting homogeneous solution was stirred at 0° C. for 1 hour, and further at r.t. for 16 hours. The colorless homogenous reaction mixture was cooled to 0° C., and methanol (200 ml) was carefully added, followed by water (200 ml). Methanol and THF were then removed under vacuum. The product was extracted with dichloromethane (3×200 ml). The combined organic extracts were washed with sat. aq. NaCl soln. (1×100 ml), dried over MgSO4, and concentrated in vacuo. The residue was purified by CC (DCM/MeOH 9:1) to yield the desired alcohol as a colorless oil.

LC-MS (A1): t_{R}=0.94 min; no ionization.

### Step 2: 3-(4-Trifluoromethyl-phenyl)-propionaldehyde

To an ice-cooled solution of 3-(4-trifluoromethyl-phenyl)-propan-1-ol (1.02 g, 5.0 mmol, 1 eq.) in DCM (11 mL), pyridinium chlorochromate (1.65 g, 7.5 mmol, 1.5 eq.) was added. The resulting black suspension was stirred at 0° C. for 10 min and further at r.t. for 15 hours. The reaction mixture was directly filtered over a plug of silicagel, eluting with DCM to yield the desired aldehyde as a yellow oil.

LC-MS (A1): t_{R}=1.00 min; no ionization.

### Step 3: 2-[3-(4-Trifluoromethyl-phenyl)-propyl]-2H-pyrazol-3-ylamine

Following the procedure described in Example 1, Step 4, but using 3-(4-trifluoromethyl-phenyl)-propionaldehyde, the desired pyrazole was obtained as a brown oil.

LC-MS (A): t_{R}=2.60 min; [M+H]⁺=270.1.

### Step 4: Title Compound

The title compound was obtained following the procedure described in Example 2, Step 2.

LC-MS (B): t_{R}=0.98 min; [M+H]⁺=516.02.

### Example 84

### 2-[1-(2,3-Dimethyl-phenyl)-1H-tetrazol-5-ylsulfanyl]-N-{2-[3-(4-isopropyl-phenyl)-propyl]-2H-pyrazol-3-yl}-acetamide

### Step 1: 2-[3-(4-Isopropyl-phenyl)-propyl]-2H-pyrazol-3-ylamine

Following the procedure described in Example 83, Steps 1 to 3, but starting from the corresponding hydrocinnamic acid, the desired pyrazole was obtained as a brown oil.

LC-MS (A): t_{R}=2.69 min; [M+H]⁺=244.1.

### Step 2

The title compound was obtained following the procedure described in Example 2, Step 2.

LC-MS (B): t_{R}=1.02 min; [M+H]⁺=489.86.

### Example 85

### 2-[1-(2,3-Dimethyl-phenyl)-1H-tetrazol-5-ylsulfanyl]-N-{2-[3-(3-fluoro-4-trifluoromethoxy-phenyl)-propyl]-2H-pyrazol-3-yl}-acetamide

### Step 1: (E)-3-(3-Fluoro-4-trifluoromethoxy-phenyl)-acrylic acid butyl ester

To a solution of 1-bromo-3-fluoro-4-(trifluoromethoxy)benzene (15.0 g, 57.9 mmol, 1.0 eq.) in DMF (250 mL) were added successively butyl acrylate (12.4 mL, 88.9 mmol, 1.5 eq.), DABCO (274 mg, 2.3 mmol, 0.04 eq.), K₂CO₃ (8.0 g, 57.9 mmol, 1.0 eq.), and Pd(OAc)₂ (260 mg, 1.2 mmol, 0.02 eq.). The resulting brown suspension was stirred at 120° C. for 2 hours, and further at r.t. for 15 hours. The mixture was extracted with ether (4×150 mL). The comb. org. layers were washed with sat. aq. NaCl soln. (2×400 mL), dried over MgSO₄, concentrated in vacuo. The residue was purified by CC (Hept/DCM 1:1) to give the desired α,β-unsaturated ester as a pale yellow oil.

LC-MS (A1): t_{R}=1.18 min; no ionization

### Step 2: 3-(3-Fluoro-4-trifluoromethoxy-phenyl)-propionic acid butyl ester

A mixture of (E)-3-(3-Fluoro-4-trifluoromethoxy-phenyl)-acrylic acid butyl ester (17.36 g, 56.7 mmol, 1 eq.) and Pd/C (1.74 g) was flushed with nitrogen. Methanol (200 mL) was carefully added. The resulting suspension was placed under vacuum, then under hydrogen. This operation was repeated two more times, and the suspension was stirred at r.t. under an H₂-atmosphere for 2 hours. The suspension was filtered over celite, and the filtrate was concentrated in vacuo to give the desired ester as pale yellow oil.

LC-MS (A1): t_{R}=1.15 min; no ionization.

### Step 3: 2-[3-(3-Fluoro-4-trifluoromethoxy-phenyl)-propyl]-2H-pyrazol-3-ylamine

Following the procedure described in Example 83, Steps 1 to 3, but starting from 3-(3-fluoro-4-trifluoromethoxy-phenyl)-propionic acid butyl ester, the desired pyrazole was obtained as a brown oil.

LC-MS (A): t_{R}=2.73 min; [M+H]⁺=303.9.

### Step 4: Title Compound

The title compound was obtained following the procedure described in Example 2, Step 2.

LC-MS (B): t_{R}=1.00 min; [M+H]⁺=550.02.

### Example 86

### N-{2-[3-(2,5-Difluoro-4-methoxy-phenyl)-propyl]-2H-pyrazol-3-yl}-2-[1-(2,3-dimethyl-phenyl)-1H-tetrazol-5-ylsulfanyl]-acetamide

### Step 1: 2-[3-(2,5-Difluoro-4-methoxy-phenyl)-propyl]-2H-pyrazol-3-ylamine

Following the procedure described in Example 83, Steps 1 to 3, but starting from the corresponding bromide, the desired pyrazole was obtained as an orange oil.

LC-MS (A): t_{R}=2.24 min; [M+H]⁺=268.0.

### Step 2

The title compound was obtained following the procedure described in Example 2, Step 2.

LC-MS (B): t_{R}=0.93 min; [M+H]⁺=514.05.

### Example 87

### N-[2-(2,3-Dihydro-benzofuran-5-ylmethyl)-2H-pyrazol-3-yl]-2-[1-(2,6-dimethyl-phenyl)-1H-tetrazol-5-ylsulfanyl]-acetamide

### Step 1: 2-(2,3-Dihydro-benzofuran-5-ylmethyl)-2H-pyrazol-3-ylamine

Following the procedure described in Example 1, Step 4, but using the corresponding aldehyde, the desired pyrazole was obtained as a pale yellow oil.

LC-MS (B): t_{R}=0.67 min; [M+H]⁺=216.16.

### Step 2

The title compound was obtained following the procedure described in Example 2, Step 2.

LC-MS (B): t_{R}=0.86 min; [M+H]⁺=461.99.

### Example 88

### N-[2-(2,4-Difluoro-3-methoxy-benzyl)-2H-pyrazol-3-yl]-2-[1-(2,6-dimethyl-phenyl)-1H-tetrazol-5-ylsulfanyl]-acetamide

### Step 1: 2-(2,4-Difluoro-3-methoxy-benzyl)-2H-pyrazol-3-ylamine

Following the procedure described in Example 1, Step 4, but using the corresponding aldehyde, the desired pyrazole was obtained as a brown oil.

LC-MS (A1): t_{R}=0.64 min; [M+H]⁺=239.99.

### Step 2

The title compound was obtained following the procedure described in Example 2, Step 2.

LC-MS (B): t_{R}=0.89 min; [M+H]⁺=486.11.

### Example 89

### 2-[1-(2,5-Dimethyl-phenyl)-1H-tetrazol-5-ylsulfanyl]-N-(2-phenethyl-2H-pyrazol-3-yl)-acetamide

The title compound was obtained following the procedure described in Example 2, Step 2, but using the corresponding pyrazole and the corresponding acid.

LC-MS (B): t_{R}=1.06 min; [M+H]⁺=434.55.

### Example 90

### 2-[1-(2,6-Dimethyl-phenyl)-1H-tetrazol-5-ylsulfanyl]-N-[2-(3-phenyl-propyl)-2H-pyrazol-3-yl]-acetamide

The title compound was obtained following the procedure described in Example 2, Step 2, but using the corresponding pyrazole and the corresponding acid.

LC-MS (B): t_{R}=0.93 min; [M+H]⁺=448.01.

### Example 91

### 2-[1-(3-Fluoro-phenyl)-1H-tetrazol-5-ylsulfanyl]-N-[2-(4-methoxy-benzyl)-2H-pyrazol-3-yl]-acetamide

### Step 1: 2-Bromo-N-[2-(4-methoxy-benzyl)-2H-pyrazol-3-yl]-acetamide

To an ice-cooled solution of 2-(4-methoxy-benzyl)-2H-pyrazol-3-ylamine (406 mg, 2.0 mmol, 1.0 eq.) in DCM (5 mL), DIPEA (0.52 ml, 3.0 mmol, 1.5 eq.) was added. A solution of bromoacetyl bromide (0.19 ml, 2.2 mmol, 1.1 eq.) in DCM (2 mL) was added dropwise. The cooling bath was removed and the brown solution was stirred at r.t. for 1.5 hours. The solution was diluted with AcOEt (75 mL), washed with sat. aq. NaHCO₃ soln. (1×40 mL), with sat. aq. NaCl soln. (1×40 mL), dried over MgSO₄, and concentrated in vacuo. The residue was purified by CC (SiO₂, AcOEt/Hept 1:1 to 6:4) to give the desired bromide as a beige solid.

LC-MS (A): t_{R}=2.60 min; [M−H]⁺=322.3.

### Step 2: 1-(3-Fluoro-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=2.60 min; [M+H]⁺=197.0.

### Step 3: Title Compound

To a solution of 2-bromo-N-[2-(4-methoxy-benzyl)-2H-pyrazol-3-yl]-acetamide (50.0 mg, 0.15 mmol, 1.00 eq.) in DMF (1.3 mL), pyridine (16 μl, 0.19 mmol, 1.25 eq.) and 1-(3-Fluoro-phenyl)-1H-tetrazole-5-thiol (30 mg, 0.15 mmol, 1.00 eq) were added in sequence. The resulting solution was stirred at r.t. for 4.5 hours. The mixture was purified by prep. HPLC and evaporated to afford the title compound.

LC-MS (B): t_{R}=0.82 min; [M+H]⁺=439.98.

### Example 92

### 2-[1-(2,6-Difluoro-phenyl)-1H-tetrazol-5-ylsulfanyl]-N-[2-(4-methoxy-benzyl)-2H-pyrazol-3-yl]-acetamide

### Step 1: 1-(2,6-Difluoro-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=2.25 min; [M−H]⁺=213.0.

### Step 2

The title compound was obtained following the procedure described in Example 91, Step 3.

LC-MS (B): t_{R}=0.81 min; [M+H]⁺=457.70.

### Example 93

### 2-[1-(2,6-Diethyl-phenyl)-1H-tetrazol-5-ylsulfanyl]-N-[2-(4-methoxy-benzyl)-2H-pyrazol-3-yl]-acetamide

### Step 1: 1-(2,6-Diethyl-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=3.09 min; [M+H]⁺=235.1.

### Step 2

The title compound was obtained following the procedure described in Example 91, Step 3.

LC-MS (B): t_{R}=0.93 min; [M+H]⁺=478.06.

### Example 94

### 2-[1-(2,6-Diisopropyl-phenyl)-1H-tetrazol-5-ylsulfanyl]-N-[2-(4-methoxy-benzyl)-2H-pyrazol-3-yl]-acetamide

### Step 1: 1-(2,6-Diisopropyl-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=3.33 min; [M+H]⁺=263.1.

### Step 2

The title compound was obtained following the procedure described in Example 91, Step 3.

LC-MS (B): t_{R}=0.98 min; [M+H]⁺=506.11.

### Example 95

### 2-[1-(2,6-Dichloro-phenyl)-1H-tetrazol-5-ylsulfanyl]-N-[2-(4-methoxy-benzyl)-2H-pyrazol-3-yl]-acetamide

### Step 1: 1-(2,6-Dichloro-phenyl)-1H-tetrazole-5-thiol

Following the procedure described in Example 1, Step 1, but starting from the corresponding isothiocyanate, the desired tetrazole was obtained as a white solid.

LC-MS (A): t_{R}=2.55 min; [M+H]⁺=248.9.

### Step 2

The title compound was obtained following the procedure described in Example 91, Step 3.

LC-MS (B): t_{R}=0.85 min; [M+H]⁺=489.62.

### Example 96

### N-[2-(4-Methoxy-benzyl)-2H-pyrazol-3-yl]-2-[1-(2,3,6-trifluoro-phenyl)-1H-tetrazol-5-ylsulfanyl]-acetamide

### Step 1: 1-(2,3,6-Trifluoro-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=2.31 min; [M+H]⁺=233.1.

### Step 2

The title compound was obtained following the procedure described in Example 91, Step 3.

LC-MS (B): t_{R}=0.82 min; [M+H]⁺=475.95.

### Example 97

### 2-[1-(2-Chloro-6-methyl-phenyl)-1H-tetrazol-5-ylsulfanyl]-N-[2-(4-methoxy-benzyl)-2H-pyrazol-3-yl]-acetamide

### Step 1: 1-(2-Chloro-6-methyl-phenyl)-1H-tetrazole-5-thiol

Following the procedure described in Example 1, Step 1, but starting from the corresponding isothiocyanate, the desired tetrazole was obtained as a white solid.

LC-MS (A): t_{R}=2.67 min; [M+H]⁺=226.8.

### Step 2

The title compound was obtained following the procedure described in Example 91, Step 3.

LC-MS (B): t_{R}=0.86 min; [M+H]⁺=469.94.

### Example 98

### N-[2-(4-Methoxy-benzyl)-2H-pyrazol-3-yl]-2-[1-(2,4,6-trimethyl-phenyl)-1H-tetrazol-5-ylsulfanyl]-acetamide

### Step 1: 1-(2,4,6-Trimethyl-phenyl)-1H-tetrazole-5-thiol

Following the procedure described in Example 1, Step 1, but starting from the corresponding isothiocyanate, the desired tetrazole was obtained as a white solid.

LC-MS (A): t_{R}=2.98 min; [M+H]⁺=221.1.

### Step 2

The title compound was obtained following the procedure described in Example 91, Step 3.

LC-MS (B): t_{R}=0.91 min; [M+H]⁺=464.12.

### Example 99

### 2-[1-(2-Fluoro-5-methyl-phenyl)-1H-tetrazol-5-ylsulfanyl]-N-[2-(4-methoxy-benzyl)-2H-pyrazol-3-yl]-acetamide

### Step 1: 1-(2-Fluoro-5-methyl-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=2.65 min; [M+H]⁺=211.1.

### Step 2

The title compound was obtained following the procedure described in Example 91, Step 3.

LC-MS (B): t_{R}=0.85 min; [M+H]⁺=453.99.

### Example 100

### 2-[1-(3-Fluoro-2-methyl-phenyl)-1H-tetrazol-5-ylsulfanyl]-N-[2-(4-methoxy-benzyl)-2H-pyrazol-3-yl]-acetamide

### Step 1: 1-(3-Fluoro-2-methyl-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=2.66 min; [M+H]⁺=211.0.

### Step 2

The title compound was obtained following the procedure described in Example 91, Step 3.

LC-MS (B): t_{R}=0.85 min; [M+H]⁺=453.91.

### Example 101

### N-[2-(4-Methoxy-benzyl)-2H-pyrazol-3-yl]-2-[1-(2,3,5-trifluoro-phenyl)-1H-tetrazol-5-ylsulfanyl]-acetamide

### Step 1: 1-(2,3,5-Trifluoro-phenyl)-1H-tetrazole-5-thiol

Following the procedure described in Example 1, Step 1, but starting from the corresponding isothiocyanate, the desired tetrazole was obtained as a white solid.

LC-MS (A): t_{R}=2.46 min; [M+H]⁺=233.1.

### Step 2

The title compound was obtained following the procedure described in Example 91, Step 3.

LC-MS (B): t_{R}=0.83 min; [M+H]⁺=475.89.

### Example 102

### 2-[1-(5-Fluoro-2-methyl-phenyl)-1H-tetrazol-5-ylsulfanyl]-N-[2-(4-methoxy-benzyl)-2H-pyrazol-3-yl]-acetamide

### Step 1: 1-(5-Fluoro-2-methyl-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=2.64 min; [M+H]⁺=211.0.

### Step 2

The title compound was obtained following the procedure described in Example 91, Step 3.

LC-MS (B): t_{R}=0.84 min; [M+H]⁺=454.01.

### Example 103

### 2-[1-(2,4-Difluoro-phenyl)-1H-tetrazol-5-ylsulfanyl]-N-[2-(4-methoxy-benzyl)-2H-pyrazol-3-yl]-acetamide

### Step 1: 1-(2,4-Difluoro-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=2.46 min; [M+H]⁺=215.0.

### Step 2

The title compound was obtained following the procedure described in Example 91, Step 3.

LC-MS (B): t_{R}=0.82 min; [M+H]⁺=457.62.

### Example 104

### N-[2-(4-Methoxy-benzyl)-2H-pyrazol-3-yl]-2-[1-(2-trifluoromethoxy-phenyl)-1H-tetrazol-5-ylsulfanyl]-acetamide

### Step 1: 1-(2-Trifluoromethoxy-phenyl)-1H-tetrazole-5-thiol

Following the procedure described in Example 1, Step 1, but starting from the corresponding isothiocyanate, the desired tetrazole was obtained as a white solid.

LC-MS (A): t_{R}=2.77 min; [M+H]⁺=263.1.

### Step 2

The title compound was obtained following the procedure described in Example 91, Step 3.

LC-MS (B): t_{R}=0.86 min; [M+H]⁺=506.01.

### Example 105

### N-[2-(4-Methoxy-benzyl)-2H-pyrazol-3-yl]-2-[1-(2,3,4-trifluoro-phenyl)-1H-tetrazol-5-ylsulfanyl]-acetamide

### Step 1: 1-(2,3,4-Trifluoro-phenyl)-1H-tetrazole-5-thiol

Following the procedure described in Example 1, Step 1, but starting from the corresponding isothiocyanate, the desired tetrazole was obtained as a white solid.

LC-MS (A): t_{R}=2.59 min; [M]⁺=232.0.

### Step 2

The title compound was obtained following the procedure described in Example 91, Step 3.

LC-MS (B): t_{R}=0.85 min; [M+H]⁺=476.11.

### Example 106

### 2-[1-(2,3-Dichloro-phenyl)-1H-tetrazol-5-ylsulfanyl]-N-[2-(4-methoxy-benzyl)-2H-pyrazol-3-yl]-acetamide

### Step 1: 1-(2,3-Dichloro-phenyl)-1H-tetrazole-5-thiol

Following the procedure described in Example 1, Step 1, but starting from the corresponding isothiocyanate, the desired tetrazole was obtained as an off-white solid.

LC-MS (A): t_{R}=2.76 min; [M+H]⁺=246.7.

### Step 2

The title compound was obtained following the procedure described in Example 91, Step 3.

LC-MS (B): t_{R}=0.88 min; [M+H]⁺=489.8.

### Example 107

### N-[2-(4-Methoxy-benzyl)-2H-pyrazol-3-yl]-2-(1-naphthalen-1-yl-1H-tetrazol-5-ylsulfanyl)-acetamide

### Step 1: 1-Naphthalen-1-yl-1H-tetrazole-5-thiol

Following the procedure described in Example 1, Step 1, but starting from the corresponding isothiocyanate, the desired tetrazole was obtained as a white solid.

LC-MS (A): t_{R}=2.81 min; [M+H]⁺=228.9.

### Step 2

The de title compound was obtained following the procedure described in Example 91, Step 3.

LC-MS (B): t_{R}=0.87 min; [M+H]⁺=471.99.

### Example 108

### 2-[1-(2-Fluoro-4-methyl-phenyl)-1H-tetrazol-5-ylsulfanyl]-N-[2-(4-methoxy-benzyl)-2H-pyrazol-3-yl]-acetamide

### Step 1: 1-(2-Fluoro-4-methyl-phenyl)-1H-tetrazole-5-thiol

Following the procedure described in Example 1, Step 1, but starting from the corresponding isothiocyanate, the desired tetrazole was obtained as an off-white solid.

LC-MS (A1): t_{R}=0.86 min; [M+H]⁺=210.92.

### Step 2

The title compound was obtained following the procedure described in Example 91, Step 3.

LC-MS (B): t_{R}=0.86 min; [M+H]⁺=454.11.

### Example 109

### 2-[1-(2-Chloro-6-trifluoromethyl-phenyl)-1H-tetrazol-5-ylsulfanyl]-N-[2-(4-methoxy-benzyl)-2H-pyrazol-3-yl]-acetamide

### Step 1: 1-(2-Chloro-6-trifluoromethyl-phenyl)-1H-tetrazole-5-thiol

Following the procedure described in Example 1, Step 1, but starting from the corresponding isothiocyanate, the desired tetrazole was obtained as a white solid.

LC-MS (A1): t_{R}=0.88 min; [M+H]⁺=280.82.

### Step 2

The title compound was obtained following the procedure described in Example 91, Step 3.

LC-MS (B): t_{R}=0.88 min; [M+H]⁺=524.02.

### Example 110

### 2-(1-Biphenyl-2-yl-1H-tetrazol-5-ylsulfanyl)-N-[2-(4-methoxy-benzyl)-2H-pyrazol-3-yl]-acetamide

### Step 1: 1-Biphenyl-2-yl-1H-tetrazole-5-thiol

Following the procedure described in Example 1, Step 1, but starting from the corresponding isothiocyanate, the desired tetrazole was obtained as a white solid.

LC-MS (A1): t_{R}=0.94 min; [M+H]⁺=254.94.

### Step 2

The title compound was obtained following the procedure described in Example 91, Step 3.

LC-MS (B): t_{R}=0.91 min; [M+H]⁺=498.14.

### Example 111

### 3-[1-(2,3-Dimethyl-phenyl)-1H-tetrazol-5-yl]-N-[2-(4-methoxy-benzyl)-2H-pyrazol-3-yl]-propionamide

### Step 1: N-(2,3-Dimethyl-phenyl)-succinamic acid ethyl ester

To an ice-cooled solution of 2,3-dimethylaniline (2.44 ml, 20.0 mmol, 1.0 eq.) and DIPEA (5.24 ml, 30.0 mmol, 1.5 eq.) in DCM (40 mL), ethyl succinyl chloride (3.62 g, 22.0 mmol, 1.1 eq.) was added dropwise. The resulting solution was stirred at r.t. for 17 hours. The solution was diluted with DCM (60 mL), washed with sat. aq. NaHCO₃ soln. (1×50 mL), with sat. aq. NaCl soln. (1×50 mL), dried over MgSO₄, and concentrated in vacuo. The residue was purified by CC (SiO₂, Hept/AcOEt 6:4) to afford the desired title compound as an off-white solid.

LC-MS (A): t_{R}=2.76 min; [M+H]⁺=250.0.

### Step 2: 3-[1-(2,3-Dimethyl-phenyl)-1H-tetrazol-5-yl]-propionic acid

To a solution of N-(2,3-Dimethyl-phenyl)-succinamic acid ethyl ester (1.25 g, 5.0 mmol, 1.0 eq.), triphenylphosphine (2.70 g, 10.0 mmol, 2.0 eq.), and trimethylsilylazide (1.38 ml, 10.0 mmol, 2.0 eq.) in THF (30 mL), DIAD (1.98 ml, 10.0 mmol, 2.0 eq.) was added dropwise. The resulting milky suspension was stirred at r.t. for 72 hours. The mixture was concentrated in vacuo and the residue was partially purified by CC (SiO₂, Hept/AcOEt 7:3 to 6:4). The resulting yellow oil was dissolved in THF (15 mL) and MeOH (4 mL). 1M aq. NaOH (6 mL) was added and the resulting solution was stirred at r.t. for 18 hours. The organic solvent was removed under vacuo. The aq. layer was diluted with water (19 ml) and washed with AcOEt (2×15 mL). The aq. layer was acidified with 6N aq. HCl and the resulting emulsion was kept at 4° C. for 6 hours. The resulting suspension was filtered to give the desired acid as a white solid. The product was used without further purification.

LC-MS (A): t_{R}=2.57 min; [M+H]⁺=247.10.

### Step 3: Title Compound

To a solution of 3-[1-(2,3-dimethyl-phenyl)-1H-tetrazol-5-yl]-propionic acid (49 mg, 0.20 mmol, 1.0 eq.) and 2-(4-methoxy-benzyl)-2H-pyrazol-3-ylamine (40 mg, 0.20 mmol, 1.0 eq.) in DMF (1.2 mL), N-(3-dimethylaminopropyl)-N′-ethylcarbodiimide hydrochloride (57 mg, 0.30 mmol, 1.5 eq.) and 4-dimethylaminopyridine (36 mg, 0.30 mmol, 1.5 eq.) were added in sequence. The mixture was stirred at r.t. for 18 hours. The mixture was purified by prep. HPLC and evaporated to afford the title compound.

LC-MS (B): t_{R}=0.85 min; [M+H]⁺=432.12.

### Example 112

### 3-[1-(2,5-Dimethyl-phenyl)-1H-tetrazol-5-yl]-N-[2-(4-methoxy-benzyl)-2H-pyrazol-3-yl]-propionamide

### Step 1: 3-[1-(2,5-Dimethyl-phenyl)-1H-tetrazol-5-yl]-propionic acid

Following the procedure described in Example 111, Steps 1 to 2, but starting from the corresponding aniline, the desired acid was obtained as a white solid.

LC-MS (A): t_{R}=2.59 min; [M+H]⁺=247.10.

### Step 2

The title compound was obtained following the procedure described in Example 111, Step 3.

LC-MS (B): t_{R}=0.86 min; [M+H]⁺=432.10.

### Example 113

### 3-[1-(2,6-Dimethyl-phenyl)-1H-tetrazol-5-yl]-N-[2-(4-methoxy-benzyl)-2H-pyrazol-3-yl]-propionamide

### Step 1: 3-[1-(2,6-Dimethyl-phenyl)-1H-tetrazol-5-yl]-propionic acid

Following the procedure described in Example 111, Steps 1 to 2, but starting from the corresponding aniline, the desired acid was obtained as a white solid.

LC-MS (A): t_{R}=2.57 min; [M+H]⁺=247.10.

### Step 2

The title compound was obtained following the procedure described in Example 111, Step 3.

LC-MS (A1): t_{R}=0.92 min; [M+H]⁺=432.04.

### Example 114

### N-[2-(4-Methoxy-benzyl)-2H-pyrazol-3-yl]-3-[1-(2,4,6-trimethyl-phenyl)-1H-tetrazol-5-yl]-propionamide

### Step 1: 3-[1-(2,4,6-Trimethyl-phenyl)-1H-tetrazol-5-yl]-propionic acid

Following the procedure described in Example 111, Steps 1 to 2, but starting from the corresponding aniline, the desired acid was obtained as a white solid.

LC-MS (A1): t_{R}=0.84 min; [M+H]⁺=261.0.

### Step 2

The title compound was obtained following the procedure described in Example 111, Step 3.

LC-MS (B): t_{R}=0.92 min; [M+H]⁺=446.19.

### Example 115

### N-[2-(4-Methoxy-benzyl)-2H-pyrazol-3-yl]-3-(1-naphthalen-1-yl-1H-tetrazol-5-yl)-propionamide

### Step 1: 3-(1-Naphthalen-1-yl-1H-tetrazol-5-yl)-propionic acid

Following the procedure described in Example 111, Steps 1 to 2, but starting from the corresponding amine, the desired acid was obtained as a white solid.

LC-MS (A1): t_{R}=0.81 min; [M+H]⁺=269.01.

### Step 2

The title compound was obtained following the procedure described in Example 111, Step 3.

LC-MS (B): t_{R}=0.86 min; [M+H]⁺=454.09.

### Example 116

### 3-[1-(2,6-Diethyl-phenyl)-1H-tetrazol-5-yl]-N-[2-(4-methoxy-benzyl)-2H-pyrazol-3-yl]-propionamide

### Step 1: 3-[1-(2,6-Diethyl-phenyl)-1H-tetrazol-5-yl]-propionic acid

Following the procedure described in Example 111, Steps 1 to 2, but starting from the corresponding aniline, the desired acid was obtained as a white solid.

LC-MS (A1): t_{R}=0.87 min; [M+H]⁺=275.05.

### Step 2

The title compound was obtained following the procedure described in Example 111, Step 3.

LC-MS (A1): t_{R}=0.99 min; [M+H]⁺=460.20.

### Example 117

### 3-[1-(2,6-Dimethoxy-phenyl)-1H-tetrazol-5-yl]-N-[2-(4-methoxy-benzyl)-2H-pyrazol-3-yl]-propionamide

### Step 1: 3-[1-(2,6-Dimethoxy-phenyl)-1H-tetrazol-5-yl]-propionic acid

Following the procedure described in Example 111, Steps 1 to 2, but starting from the corresponding aniline, the desired acid was obtained as a white solid.

LC-MS (A2): t_{R}=0.60 min; [M+H]⁺=279.29.

### Step 2

The title compound was obtained following the procedure described in Example 111, Step 3.

LC-MS (A1): t_{R}=0.89 min; [M+H]⁺=464.13.

### Example 118

### N-[2-(4-Isopropyl-benzyl)-2H-pyrazol-3-yl]-3-(1-phenyl-1H-tetrazol-5-yl)-propionamide

### Step 1: 3-(1-Phenyl-1H-tetrazol-5-yl)-propionic acid

Following the procedure described in Example 111, Steps 1 to 2, but starting from the corresponding aniline, the desired acid was obtained as a pale orange solid.

LC-MS (A): t_{R}=2.19 min; [M+H]⁺=219.0

### Step 2

The title compound was obtained following the procedure described in Example 111, Step 3, but starting from 2-(4-isopropyl-benzyl)-2H-pyrazol-3-ylamine.

LC-MS (B): t_{R}=0.89 min; [M+H]⁺=416.07.

### Example 119

### 3-[1-(2,3-Dimethyl-phenyl)-1H-tetrazol-5-yl]-N-[2-(4-isopropyl-benzyl)-2H-pyrazol-3-yl]-propionamide

The title compound was obtained following the procedure described in Example 118, Step 2, but starting from the corresponding acid.

LC-MS (B): t_{R}=0.94 min; [M+H]⁺=444.14.

### Example 120

### 3-[1-(2,5-Dimethyl-phenyl)-1H-tetrazol-5-yl]-N-[2-(4-isopropyl-benzyl)-2H-pyrazol-3-yl]-propionamide

The title compound was obtained following the procedure described in Example 118, Step 2, but starting from the corresponding acid.

LC-MS (B): t_{R}=0.95 min; [M+H]⁺=444.15.

### Example 121

### 3-[1-(2,6-Dimethyl-phenyl)-1H-tetrazol-5-yl]-N-[2-(4-isopropyl-benzyl)-2H-pyrazol-3-yl]-propionamide

The desired title compound was obtained following the procedure described in Example 118, Step 2, but starting from the corresponding acid.

LC-MS (A1): t_{R}=1.04 min; [M+H]⁺=444.08.

### Example 122

### 3-[1-(2,3-Dimethyl-phenyl)-1H-tetrazol-5-yl]-N-[2-(4-methoxy-benzyl)-5-methyl-2H-pyrazol-3-yl]-propionamide

### Step 1: 2-(4-Methoxy-benzyl)-5-methyl-2H-pyrazol-3-ylamine

To a solution of hydrazine monohydrate (0.51 ml, 10.5 mmol, 1.05 eq.) in THF (2 mL), crotononitrile (mixture of cis and trans) (0.82 ml, 10.0 mmol, 1.00 eq.) was added dropwise. The mixture was stirred at 40° C. for 2 hours. The mixture was allowed to cool to r.t. and anisaldehyde (1.21 ml, 10.0 mmol, 1.00 eq.) was added dropwise. The mixture was stirred at 40° C. for 2 hours. The mixture was concentrated in vacuo. The resulting yellow oil was dissolved in iPrOH (8 mL). Sodium tert-butylate (991 mg, 10.0 mmol, 1.00 eq.) was added and the mixture was stirred at 110° C. for 4 hours. The mixture was allowed to cool to r.t. and diluted with water (50 mL). The mixture was extracted with Et₂O (3×50 mL). The comb. org. phases were extracted with 1N aq. HCl (2×30 mL). The comb. aq. phases were basified to pH 14 with 50% aq. NaOH soln. and extracted with Et₂O (3×50 mL). The comb. org. phases were dried over MgSO₄ and concentrated in vacuo to give a yellow solid. The product was used without further purification.

LC-MS (B): t_{R}=0.70 min; [M+H]⁺=218.30.

### Step 2

The title compound was obtained following the procedure described in Example 118, Step 3.

LC-MS (A1): t_{R}=0.92 min; [M+H]⁺=446.15.

### Example 123

### 3-[1-(2,6-Diethyl-phenyl)-1H-tetrazol-5-yl]-N-[2-(4-methoxy-benzyl)-5-methyl-2H-pyrazol-3-yl]-propionamide

The title compound was obtained following the procedure described in Example 122, Steps 1 and 2, but using the corresponding acid (Example 116, Step 1).

LC-MS (A1): t_{R}=1.00 min; [M+H]⁺=474.04.

### Example 124

### 2-[1-(2,3-Dimethyl-phenyl)-1H-tetrazol-5-ylsulfanyl]-N-[2-(4-methoxy-benzyl)-5-methyl-2H-pyrazol-3-yl]-acetamide

The title compound was obtained following the procedure described in Example 122, Step 2, but using the corresponding acid (Example 1, Step 2).

LC-MS (A1): t_{R}=0.98 min; [M+H]⁺=464.11.

### Example 125

### 3-[1-(2,3-Dimethyl-phenyl)-1H-tetrazol-5-yl]-N-[2-(4-methoxy-benzyl)-4-methyl-2H-pyrazol-3-yl]-propionamide

### Step 1: 2-(4-Methoxy-benzyl)-4-methyl-2H-pyrazol-3-ylamine

Following the procedure described in Example 122, Step 1, but starting from methacrylonitrile, the desired pyrazole was obtained as a pale yellow solid.

LC-MS (B): t_{R}=0.71 min; [M+H]⁺=218.26.

### Step 2

The title compound was obtained following the procedure described in Example 111, Step 3.

LC-MS (A1): t_{R}=0.92 min; [M+H]⁺=446.16.

### Example 126

### 3-[1-(2,6-Diethyl-phenyl)-1H-tetrazol-5-yl]-N-[2-(4-methoxy-benzyl)-4-methyl-2H-pyrazol-3-yl]-propionamide

The title compound was obtained following the procedure described in Example 125, Step 2, but starting from the corresponding acid.

LC-MS (A1): t_{R}=1.00 min; [M+H]⁺=474.05.

### Example 127

### 2-[1-(2,3-Dimethyl-phenyl)-1H-tetrazol-5-ylsulfanyl]-N-[2-(4-methoxy-benzyl)-4-methyl-2H-pyrazol-3-yl]-acetamide

The title compound was obtained following the procedure described in Example 125, Step 2, but starting from the corresponding acid.

LC-MS (A1): t_{R}=0.98 min; [M+H]⁺=464.08.

### Example 128

### 2-[1-(2,5-Dimethyl-phenyl)-1H-tetrazol-5-ylsulfanyl]-N-[2-(4-methoxy-benzyl)-2H-pyrazol-3-yl]-propionamide

### Step 1: 2-[1-(2,5-Dimethyl-phenyl)-1H-tetrazol-5-ylsulfanyl]-propionic acid ethyl ester

To a solution of 1-(2,5-dimethyl-phenyl)-1H-tetrazole-5-thiol (1.03 g, 5.0 mmol, 1.00 eq.) in DMSO (13 mL), pyridine (0.50 mL, 6.25 mmol, 1.25 eq.) and ethyl 2-bromopropionate (0.65 mL, 5.0 mmol, 1.00 eq.) were added in sequence. The resulting solution was stirred at r.t. for 2.5 hours and further at 60° C. for 2 hours. Pyridine (0.50 mL, 6.25 mmol, 1.25 eq.) and ethyl 2-bromopropionate (0.65 ml, 5.0 mmol, 1.00 eq.) were added and the resulting mixture was heated up to 80° C. for 2.5 hours. The solution was diluted with AcOEt and washed successively with water and sat. aq. NaCl solution. The org. layer was dried over MgSO₄ and concentrated in vacuo. The crude product was purified by CC (SiO₂, Hept/AcOEt 9:1 to 8:2) to afford the desired ester as a colorless oil.

LC-MS (A): t_{R}=3.45 min; [M+H]⁺=307.3.

### Step 2: 2-[1-(2,5-Dimethyl-phenyl)-1H-tetrazol-5-ylsulfanyl]-propionic acid

To a solution of 2-[1-(2,5-Dimethyl-phenyl)-1H-tetrazol-5-ylsulfanyl]-propionic acid ethyl ester (1.56 g, 5.1 mmol, 1.0 eq.) in THF (14 mL) and MeOH (4.5 mL), 1M aq. NaOH solution (6 mL) was added. The solution was stirred at r.t. for 17 hours. The solution was concentrated in vacuo. The residue was dissolved in 1M aq. NaOH solution (20 mL) and water (20 mL). The solution was acidified with 1N aq. HCl solution and the resulting emulsion was kept at 4° C. overnight. The resulting emulsion was extracted twice with AcOEt. The comb. org. phases were dried over MgSO₄ then concentrated in vacuo to give the desired acid as a colorless oil that solidifies upon standing. The product was used without further purification.

LC-MS (A): t_{R}=2.94 min; [M+H]⁺=279.1.

### Step 3: Title Compound

The title compound was obtained following the procedure described in Example 45, Step 2.

LC-MS (A1): t_{R}=1.08 min; [M+H]⁺=464.28.

### Example 129

### 2-[1-(2,5-Dimethyl-phenyl)-1H-tetrazol-5-ylsulfanyl]-N-[2-(4-methoxy-benzyl)-2H-pyrazol-3-yl]-butyramide

### Step 1: 2-[1-(2,5-Dimethyl-phenyl)-1H-tetrazol-5-ylsulfanyl]-butyric acid

Following the procedure described in Example 128, Steps 1 to 2, but starting from methyl 2-bromobutyrate, the desired acid was obtained as a colorless oil.

LC-MS (A): t_{R}=3.04 min; [M+H]⁺=293.2.

### Step 2

The title compound was obtained following the procedure described in Example 45, Step 2.

LC-MS (A1): t_{R}=1.11 min; [M+H]⁺=478.21.

### Example 130

### 2-[1-(2,5-Dimethyl-phenyl)-1H-tetrazol-5-ylsulfanyl]-N-[2-(4-methoxy-phenyl)-2H-pyrazol-3-yl]-acetamide

### Step 1: 5-Amino-1-(4-methoxy-phenyl)-1H-pyrazole-4-carboxylic acid ethyl ester

To a solution of 4-methoxyphenylhydrazine hydrochloride (3.00 g, 17 mmol, 1.0 eq.) in EtOH (15 mL) was added ethyl 2-cyano-3-ethoxyacrylate (2.97 g, 17 mmol, 1.0 eq.). The suspension was refluxed for 18 hours. After the reaction mixture reached r.t. the solid was filtered off, washed with EtOH and the filtrate was concentrated to a brown oil. The residue was purified by an automated chromatography system (Biotage, eluent: AcOEt/hexane).

LC-MS (A1): t_{R}=0.86 min; [M+H]⁺=261.99.

### Step 2: 2-(4-Methoxy-phenyl)-2H-pyrazol-3-ylamine

A solution of 5-amino-1-(4-methoxy-phenyl)-1H-pyrazole-4-carboxylic acid ethyl ester (1.38 g, 5.3 mmol) in HCl (32%, 20 mL) was stirred at 90° C. for 24 hours, then the reaction mixture was basified with 4 N aq. NaOH soln. The inorganic layer was extracted with DCM (3×) and the combined org. phases were dried over MgSO4, and concentrated in vacuo to furnish a yellow oil which was used in the next step without further purification.

LC-MS (A1): t_{R}=0.54 min; [M+H]⁺=189.99.

### Step 3

To a solution 2-(4-methoxy-phenyl)-2H-pyrazol-3-ylamine (60 mg, 0.315 mmol, 1 eq.) in DCM/THF (2.0 mL, 0.3 mL) was added DIPEA (0.3 mL, 1.75 mmol, 5.5 eq.) followed by [1-(2,5-dimethyl-phenyl)-1H-tetrazol-5-ylsulfanyl]-acetic acid (80 mg, 0.315 mmol, 1 eq.) and HATU (298 mg, 0.78 mmol, 2.5 eq.). After the suspension was stirred at r.t for 1 hour, the reaction mixture was diluted with AcOEt and washed with 1N aq. NaHSO₄ soln. and sat. aq. NaHSO₃ soln. The org. phase was concentrated in vacuo. The residue was purified by an automated chromatography system (Biotage, eluent: AcOEt/hexane) to yield title compound as a colorless oil.

LC-MS (A1): t_{R}=0.97 min; [M+H]⁺=436.01.

### Example 131

### 2-[1-(2,3-Dimethyl-phenyl)-1H-tetrazol-5-ylsulfanyl]-N-(2-phenyl-2H-pyrazol-3-yl)-acetamide

### Step 1: 2-Phenyl-2H-pyrazol-3-ylamine

Following the procedure described in Example 130, Steps 1 and 2, but starting from the corresponding hydrazine, the desired pyrazole was obtained as a yellow oil.

LC-MS (A2): t_{R}=0.21 min; [M+H]⁺=261.99.

### Step 2

The title compound was obtained following the procedure described in Example 130, Step 3, but using the corresponding 2-phenyl-2H-pyrazol-3-ylamine and [1-(2,3-dimethyl-phenyl)-1H-tetrazol-5-ylsulfanyl]-acetic acid (Example 1, Steps 2 to 3).

LC-MS (A2): t_{R}=0.69 min; [M+H]⁺=406.21.

### Example 132

### N-[2-(3,4-Dichloro-phenyl)-2H-pyrazol-3-yl]-2-[1-(2,3-dimethyl-phenyl)-1H-tetrazol-5-ylsulfanyl]-acetamide

### Step 1: 2-(3,4-Dichloro-phenyl)-2H-pyrazol-3-ylamine

Following the procedure described in Example 130, Steps 1 and 2, but starting from the corresponding hydrazine hydrochloride, the desired pyrazole was obtained as an off-white solid.

LC-MS (A2): t_{R}=0.50 min; [M+H]⁺=228.17.

### Step 2

To a solution of 2-(3,4-dichloro-phenyl)-2H-pyrazol-3-ylamine (60 mg, 0.315 mmol, 1 eq.) in DCM/THF (2.0 mL, 0.3 mL) was added DIPEA (0.3 mL, 1.75 mmol, 5.5 eq.) followed by [1-(2,3-dimethyl-phenyl)-1H-tetrazol-5-ylsulfanyl]-acetic acid (80 mg, 0.315 mmol, 1 eq.) and HATU (298 mg, 0.78 mmol, 2.5 eq.). After the suspension was stirred at r.t. for 1 hour, the reaction mixture was diluted with AcOEt and washed with 1N aq. NaHSO₄ soln. and sat. aq. NaHSO₃ soln. The org. phase was concentrated in vacuo, purified by prep. HPLC and evaporated to afford the title compound.

LC-MS (A2): t_{R}=0.77 min; [M+H]⁺=474.1.

### Example 133

### 2-[1-(2,3-Dimethyl-phenyl)-1H-tetrazol-5-ylsulfanyl]-N-[2-(4-trifluoromethoxy-phenyl)-2H-pyrazol-3-yl]-acetamide

### Step 1: 2-(4-Trifluoromethoxy-phenyl)-2H-pyrazol-3-ylamine

Following the procedure described in Example 130, Step 2, but using the corresponding pyrazole-4-carboxylic acid ethyl ester, the desired pyrazole was obtained as an off-white solid.

LC-MS (A2): t_{R}=0.48 min; [M+H]⁺=246.17.

### Step 2

The title compound was obtained following the procedure described in Example 132, Step 2, using 2-(4-trifluoromethoxy-phenyl)-2H-pyrazol-3-ylamine.

LC-MS (A2): t_{R}=0.77 min; [M+H]⁺=490.14.

### Example 134

### 2-[1-(2,3-Dimethyl-phenyl)-1H-tetrazol-5-ylsulfanyl]-N-[2-(4-isopropyl-phenyl)-2H-pyrazol-3-yl]-acetamide

### Step 1: 2-(4-Isopropyl-phenyl)-2H-pyrazol-3-ylamine

Following the procedure described in Example 132, Steps 1 and 2, but starting from the corresponding hydrazine hydrochloride, the desired pyrazole was obtained as a brown solid

LC-MS (A2): t_{R}=0.45 min; [M+H]⁺=202.31.

### Step 2

The title compound was obtained following the procedure described in Example 132, Step 2, using 2-(4-isopropyl-phenyl)-2H-pyrazol-3-ylamine.

LC-MS (A2): t_{R}=0.78 min; [M+H]⁺=448.2.

### Example 135

### 2-[1-(2,3-Dimethyl-phenyl)-1H-tetrazol-5-ylsulfanyl]-N-[2-(4-fluoro-phenyl)-2H-pyrazol-3-yl]-acetamide

### Step 1: 2-(4-Fluoro-phenyl)-2H-pyrazol-3-ylamine

Following the procedure described in Example 130, Steps 1 and 2, but starting from the corresponding hydrazine hydrochloride, the desired pyrazole was obtained as a yellow oil.

LC-MS (A2): t_{R}=0.23 min; [M+H]⁺=178.36.

### Step 2

The title compound was obtained following the procedure described in Example 132, Step 2, using 2-(4-fluoro-phenyl)-2H-pyrazol-3-ylamine.

LC-MS (A2): t_{R}=0.71 min; [M+H]⁺=424.13.

### Example 136

### 2-[1-(2,3-Dimethyl-phenyl)-1H-tetrazol-5-ylsulfanyl]-N-[2-(3-methoxy-phenyl)-2H-pyrazol-3-yl]-acetamide

### Step 1: 2-(3-Methoxy-phenyl)-2H-pyrazol-3-ylamine

Following the procedure described in Example 130, Steps 1 and 2, but starting from the corresponding hydrazine hydrochloride, the desired pyrazole was obtained as a brown oil.

LC-MS (A2): t_{R}=0.29 min; [M+H]⁺=190.3.

### Step 2

The title compound was obtained following the procedure described in Example 132, Step 2, but using 2-(3-methoxy-phenyl)-2H-pyrazol-3-ylamine.

LC-MS (A2): t_{R}=0.71 min; [M+H]⁺=436.15.

### Example 137

### 2-[1-(2,5-Dimethyl-phenyl)-1H-tetrazol-5-ylsulfanyl]-N-(2-phenyl-2H-pyrazol-3-yl)-acetamide

The title compound was obtained following the procedure described in Example 131, Steps 1 to 2, but using the corresponding [1-(2,5-dimethyl-phenyl)-1H-tetrazol-5-ylsulfanyl]-acetic acid (Example 46, Step 1).

LC-MS (A2): t_{R}=0.7 min; [M+H]⁺=406.22.

### Example 138

### 3-[1-(2,6-Dimethyl-phenyl)-1H-tetrazol-5-yl]-N-[2-(4-methoxy-phenyl)-2H-pyrazol-3-yl]-propionamide

The title compound was obtained following the procedure described in Example 132, Step 2, using 2-(4-methoxy-phenyl)-2H-pyrazol-3-ylamine (Example 130, Steps 1 and 2) and 3-[1-(2,6-dimethyl-phenyl)-1H-tetrazol-5-yl]-propionic acid (Example 113, Step 1).

LC-MS (A2): t_{R}=0.65 min; [M+H]⁺=417.74.

### Example 139

### N-[2-(4-Methoxy-phenyl)-2H-pyrazol-3-yl]-3-[1-(2,4,6-trimethyl-phenyl)-1H-tetrazol-5-yl]-propionamide

The title compound was obtained following the procedure described in Example 132, Step 2, using 2-(4-methoxy-phenyl)-2H-pyrazol-3-ylamine (Example 130, Step 1 and 2) and 3-[1-(2,4,6-trimethyl-phenyl)-1H-tetrazol-5-yl]-propionic acid (Example 114, Step 1).

LC-MS (A2): t_{R}=0.69 min; [M+H]⁺=432.24.

### Example 140

### N-[2-(4-Methoxy-phenyl)-2H-pyrazol-3-yl]-3-(1-naphthalen-1-yl-1H-tetrazol-5-yl)-propionamide

The title compound was obtained following the procedure described in Example 132, Step 2, using 2-(4-methoxy-phenyl)-2H-pyrazol-3-ylamine (Example 130, Steps 1 and 2) and 3-(1-naphthalen-1-yl-1H-tetrazol-5-yl)-propionic acid (Example 115, Step 1).

LC-MS (A2): t_{R}=0.66 min; [M+H]⁺=440.28.

### Example 141

### 2-[1-(2,3-Dimethyl-phenyl)-1H-tetrazol-5-ylsulfanyl]-N-[2-(4-phenoxy-phenyl)-2H-pyrazol-3-yl]-acetamide

### Step 1: 2-(4-Phenoxy-phenyl)-2H-pyrazol-3-ylamine

Following the procedure described in Example 130, Steps 1 and 2, but starting from the corresponding hydrazine hydrochloride, the desired pyrazole was obtained as an orange oil.

LC-MS (A2): t_{R}=0.52 min; [M+H]⁺=252.19.

### Step 2

The title compound was obtained following the procedure described in Example 132, Step 2, but using the corresponding pyrazole and [1-(2,3-dimethyl-phenyl)-1H-tetrazol-5-ylsulfanyl]-acetic acid (Example 1, Steps 2 and 3).

LC-MS (A2): t_{R}=0.79 min; [M+H]⁺=497.83.

### Example 142

### 2-[1-(2,3-Dimethyl-phenyl)-1H-tetrazol-5-ylsulfanyl]-N-(2-p-tolyl-2H-pyrazol-3-yl)-acetamide

### Step 1: p-Tolyl-hydrazine

To commercially available p-toluidine (3.00 g, 0.028 mol)) was added concentrated HCl 37% (10 mL). The suspension was stirred at r.t. for 18 hours, then cooled to 0° C. and a solution of sodium nitrite (2.25 g, 0.033 mol, 1.16 eq.) in water (8.5 mL) was added dropwise at 0-5° C. After stirring for 1.5 hours at this temperature, a solution of tin(II)chloride dihydrate (26.6 g, 0.118 mol, 4.2 eq.) in HCl 37% (21 mL) was added. The reaction mixture was allowed to warm to r.t. and stored at 4° C. for 18 hours. The resulting precipitate was collected by filtration, washed with water (16 mL) and Et₂O (10 mL) and dried in vacuo. The solid HCl salt was basified with 3N aq. NaOH soln., then the free base was extracted into Et₂O and the solvent was removed in vacuo.

LC-MS (A2): t_{R}=0.15 min; [M+H]⁺=123.18.

### Step 2: 2-p-Tolyl-2H-pyrazol-3-ylamine

Following the procedure described in Example 130, Steps 1 and 2.

LC-MS (A2): t_{R}=0.30 min; [M+H]⁺=174.13.

### Step 3: Title Compound

The title compound was obtained following the procedure described in Example 132, Step 2, but using 2-p-tolyl-2H-pyrazol-3-ylamine and [1-(2,3-dimethyl-phenyl)-1H-tetrazol-5-ylsulfanyl]-acetic acid (Example 1, Steps 2 and 3)

LC-MS (A2): t_{R}=0.72 min; [M+H]⁺=420.07.

### Example 143

### 2-[1-(2,3-Dimethyl-phenyl)-1H-tetrazol-5-ylsulfanyl]-N-[2-(2-methoxy-phenyl)-2H-pyrazol-3-yl]-acetamide

### Step 1: 2-(2-Methoxy-phenyl)-2H-pyrazol-3-ylamine

Following the procedure described in Example 130, Steps 1 and 2, but starting from the corresponding hydrazine hydrochloride, the desired pyrazole was obtained as a yellow oil.

LC-MS (A2): t_{R}=0.22 min; [M+H]⁺=190.34.

### Step 2

The title compound was obtained following the procedure described in Example 132, Step 2, but using 2-(2-methoxy-phenyl)-2H-pyrazol-3-ylamine and [1-(2,3-dimethyl-phenyl)-1H-tetrazol-5-ylsulfanyl]-acetic acid (Example 1, Steps 2 and 3).

LC-MS (A2): t_{R}=0.71 min; [M+H]⁺=436.21.

### Example 144

### 3-[1-(2,3-Dimethyl-phenyl)-1H-tetrazol-5-yl]-N-[2-(3′-fluoro-biphenyl-3-yl)-2H-pyrazol-3-yl]-propionamide

### Step 1: 2-(3′-Fluoro-biphenyl-3-yl)-2H-pyrazol-3-ylamine

Following the procedure described in Example 130, Steps 1 and 2, but starting from the corresponding hydrazine hydrochloride, the desired pyrazole was obtained as a yellow oil.

LC-MS (A2): t_{R}=0.55 min; [M+H]⁺=254.21.

### Step 2

The title compound was obtained following the procedure described in Example 132, Step 2, but using 2-(3′-fluoro-biphenyl-3-yl)-2H-pyrazol-3-ylamine and 3-[1-(2,3-Dimethyl-phenyl)-1H-tetrazol-5-yl]-propionic acid (Example 111, Steps 1 and 2)

LC-MS (A2): t_{R}=0.77 min; [M+H]⁺=481.86

### Example 145

### 2-[1-(2,3-Dimethyl-phenyl)-1H-tetrazol-5-ylsulfanyl]-N-[2-(3-fluoro-4-methoxy-phenyl)-2H-pyrazol-3-yl]acetamide

### Step 1: 2-(3-Fluoro-4-methoxy-phenyl)-2H-pyrazol-3-ylamine

Following the procedure described in Example 130, Steps 1 and 2, but starting from the corresponding hydrazine hydrochloride, the desired pyrazole was obtained as a yellow solid.

LC-MS (A2): t_{R}=0.32 min; [M+H]⁺=208.33.

### Step 2

The title compound was obtained following the procedure described in Example 132, Step 2, but using the corresponding pyrazole and [1-(2,3-dimethyl-phenyl)-1H-tetrazol-5-ylsulfanyl]-acetic acid (Example 1, Steps 2 and 3).

LC-MS (A2): t_{R}=0.72 min; [M+H]⁺=454.21.

### Example 146

### 2-[1-(2,3-Dimethyl-phenyl)-1H-tetrazol-5-ylsulfanyl]-N-[2-(3′-fluoro-biphenyl-3-yl)-2H-pyrazol-3-yl]-acetamide

The title compound was obtained following the procedure described in Example 132, Step 2, but using 2-(3′-fluoro-biphenyl-3-yl)-2H-pyrazol-3-ylamine (Example 144, Step 1) and [1-(2,3-dimethyl-phenyl)-1H-tetrazol-5-ylsulfanyl]-acetic acid (Example 1, Steps 2 and 3).

LC-MS (A2): t_{R}=0.8 min; [M+H]⁺=500.3.

### Example 147

### 2-[1-(2,3-Dimethyl-phenyl)-1H-tetrazol-5-ylsulfanyl]-N-[2-(6-methoxy-pyridin-3-yl)-2H-pyrazol-3-yl]-acetamide

### Step 1: (6-Methoxy-pyridin-3-yl)-hydrazine

Following the procedure described in Example 142, Step 1, but starting from the corresponding aniline, the desired hydrazine was obtained as a brown solid.

LC-MS (A2): t_{R}=0.1 min; [M+H]⁺=140.2.

### Step 2: 5-Amino-1-(6-methoxy-pyridin-3-yl)-1H-pyrazole-4-carboxylic acid ethyl ester

Following the procedure described in Example 130, Step 1, but starting from the corresponding hydrazine hydrochloride, the desired ester was obtained as a brown oil.

LC-MS (A2): t_{R}=0.54 min; [M+H]⁺=236.21.

### Step 3: 2-(6-Methoxy-pyridin-3-yl)-2H-pyrazol-3-ylamine

To a solution of 5-amino-1-(6-methoxy-pyridin-3-yl)-1H-pyrazole-4-carboxylic acid ethyl ester (0.97 g, 3.7 mmol) in MeOH (10.0 mL) was added 2 N aq. NaOH soln. (9.0 mL), The reactionmixture was stirred at r.t. for 5 hours. To the reaction mixture was added 37% HCl (5.5 mL) and stirred at 60° C. for 5 hours. After the reaction mixture reached r.t. the reactionmixture was basified with 12.5 N aq. NaOH-soln. The inorg, phase was extracted with DCM (2×), the combined organic layers were dried with MgSO₄ and concentrated in vacuo. The residue was purified on an automated chromatography system (Biotage, eluent: DCM/MeOH or AcOEt/hexane).

LC-MS (A2): t_{R}=0.25 min; [M+H]⁺=191.36.

### Step 4: Title Compound

The title compound was obtained following the procedure described in Example 132, Step 2, but using the pyrazole and [1-(2,3-dimethyl-phenyl)-1H-tetrazol-5-ylsulfanyl]-acetic acid (Example 1, Steps 2 and 3).

LC-MS (A2): t_{R}=0.68 min; [M+H]⁺=437.25.

### Example 148

### N-[2-(7-Chloro-quinolin-4-yl)-2H-pyrazol-3-yl]-2-[1-(2,3-dimethyl-phenyl)-1H-tetrazol-5-ylsulfanyl]-acetamide

### Step 1: 2-(7-Chloro-quinolin-4-yl)-2H-pyrazol-3-ylamine

Following the procedure described in Example 147, Steps 2 and 3, starting from the corresponding hydrazine, the desired pyrazole was obtained as a yellow solid.

LC-MS (A2): t_{R}=0.44 min; [M+H]⁺=245.11.

### Step 2

The title compound was obtained following the procedure described in Example 132, Step 2, but using 2-(7-chloro-quinolin-4-yl)-2H-pyrazol-3-ylamine and [1-(2,3-dimethyl-phenyl)-1H-tetrazol-5-ylsulfanyl]-acetic acid (Example 1, Steps 2 and 3).

LC-MS (A2): t_{R}=0.74 min; [M+H]⁺=491.07.

### Example 149

### 2-[1-(2,3-Dimethyl-phenyl)-1H-tetrazol-5-ylsulfanyl]-N-(2-pyridin-4-yl-2H-pyrazol-3-yl)-acetamide

### Step 1: 2-Pyridin-4-yl-2H-pyrazol-3-ylamine

Following the procedure described in Example 147, Steps 2 and 3, starting from the corresponding hydrazine hydrochloride, the desired pyrazole was obtained as a yellow solid.

LC-MS (A2): t_{R}=0.10 min; [M+H]⁺=161.12.

### Step 2

The title compound was obtained following the procedure described in Example 132, Step 2, but using 2-pyridin-4-yl-2H-pyrazol-3-ylamine and [1-(2,3-dimethyl-phenyl)-1H-tetrazol-5-ylsulfanyl]-acetic acid (Example 1, Steps 2 and 3).

LC-MS (A2): t_{R}=0.53 min; [M+H]⁺=407.16.

### Example 150

### N-[2-(4-Methoxy-phenyl)-2H-pyrazol-3-yl]-2-[1-(2,4,6-trimethyl-phenyl)-1H-tetrazol-5-ylsulfanyl]-acetamide

### Step 1: 2-Bromo-N-[2-(4-methoxy-phenyl)-2H-pyrazol-3-yl]-acetamide

To a solution of 2-(4-methoxy-phenyl)-2H-pyrazol-3-ylamine (2.0 g, 0.011 mmol, 1.0 eq.) in DCM (28 mL) at 0° C. was added DIPEA (2.8 mL, 0.016 mmol, 1.5 eq.) and a solution of bromoacetyl bromide (1.1 mL, 0.013 mol, 1.2 eq.) in DCM (11 mL) was added dropwise during 20 min. The cooling bath was removed and the brown solution stirred at r.t. for 2.5 hours. The solution was washed with once with sat. aq. NaHCO₃ soln. and once with brine. The org. phase was concentrated in vacuo and the residue was purified by automated chromatography system (Biotage, eluent: AcOEt/hexane).

LC-MS (A2): t_{R}=0.49 min; [M+H]⁺=310.13.

### Step 2

The title compound was obtained following the procedure described in Example 91, Step 3, using 2-bromo-N-[2-(4-methoxy-phenyl)-2H-pyrazol-3-yl]-acetamide (Example 150, Step 1) and 1-(2,4,6-trimethyl-phenyl)-1H-tetrazole-5-thiol (Example 98, Step 1).

LC-MS (B): t_{R}=0.91 min; [M+H]⁺=450.09.

### Example 151

### N-[2-(4-Methoxy-phenyl)-2H-pyrazol-3-yl]-2-(1-naphthalen-1-yl-1H-tetrazol-5-ylsulfanyl)-acetamide

The title compound was obtained following the procedure described in Example 91, Step 3, using 2-bromo-N-[2-(4-methoxy-phenyl)-2H-pyrazol-3-yl]-acetamide (Example 150, Step 1) and 1-naphthalen-1-yl-1H-tetrazole-5-thiol (Example 107, Step 1).

LC-MS (B): t_{R}=0.88 min; [M+H]⁺=457.83

### Example 152

### 2-[1-(2,6-Dichloro-phenyl)-1H-tetrazol-5-ylsulfanyl]-N-[2-(4-methoxy-phenyl)-2H-pyrazol-3-yl]-acetamide

The title compound was obtained following the procedure described in Example 91, Step 3, using 2-bromo-N-[2-(4-methoxy-phenyl)-2H-pyrazol-3-yl]-acetamide (Example 150, Step 1) and 1-(2,6-dichloro-phenyl)-1H-tetrazole-5-thiol (Example 95, Step 1).

LC-MS (B): t_{R}=0.86 min; [M+H]⁺=476.01.

### Example 153

### 2-[1-(2,6-Dimethyl-phenyl)-1H-tetrazol-5-ylsulfanyl]-N-[2-(4-methoxy-phenyl)-2H-pyrazol-3-yl]-acetamide

The title compound was obtained following the procedure described in Example 91, Step 3, using 2-bromo-N-[2-(4-methoxy-phenyl)-2H-pyrazol-3-yl]-acetamide (Example 150, Steps 1) and 1-(2,6-dimethyl-phenyl)-1H-tetrazole-5-thiol (according to Example 1, Step 1, but starting from the corresponding isothiocyanate).

LC-MS (B): t_{R}=0.87 min; [M+H]⁺=436.15.

### Example 154

### 2-[1-(2,3-Dimethyl-phenyl)-1H-tetrazol-5-ylsulfanyl]-N-(5-methyl-2-phenyl-2H-pyrazol-3-yl)-acetamide

The title compound was obtained following the procedure described in Example 2, Step 2, but using the corresponding commercially available 5-amino-3-methyl-1-phenylpyrazol and [1-(2,3-dimethyl-phenyl)-1H-tetrazol-5-ylsulfanyl]-acetic acid (Example 1, Steps 2 and 3).

LC-MS (B): t_{R}=0.89 min; [M+H]⁺=420.16.

### Example 155

### N-[2-(4-Methoxy-benzyl)-2H-pyrazol-3-yl]-2-(1-p-tolyl-1H-tetrazol-5-ylsulfanyl)-acetamide

### Step 1: (1-p-Tolyl-1H-tetrazol-5-ylsulfanyl)-acetic acid

Following the procedure described in Example 1, Steps 1 to 3, but using the corresponding isothiocyanate, the desired acid was obtained as a white solid.

LC-MS (A): t_{R}=2.67 min; [M+H]⁺=251.32.

### Step 2

The title compound was obtained following the procedure described in Example 45, Step 2.

LC-MS (B): t_{R}=0.84 min; [M+H]⁺=436.13.

### II. Biological Assays

### In Vitro Assay

The orexin receptor antagonistic activity of the compounds of formula (I) is determined in accordance with the following experimental method.

Chinese hamster ovary (CHO) cells expressing the human orexin-1 receptor and the human orexin-2 receptor, respectively, are grown in culture medium (Ham F-12 with L-Glutamine) containing 300 μg/ml G418, 100 U/ml penicillin, 100 μg/ml streptomycin and 10% heat inactivated fetal calf serum (FCS). The cells are seeded at 20,000 cells/well into 384-well black clear bottom sterile plates (Greiner). The seeded plates are incubated overnight at 37° C. in 5% CO₂. Human orexin-A as an agonist is prepared as 1 mM stock solution in MeOH:water (1:1), diluted in HBSS containing 0.1% bovine serum albumin (BSA), NaHCO₃: 0.375 g/l and 20 mM HEPES for use in the assay at a final concentration of 3 nM.

Antagonists are prepared as 10 mM stock solution in DMSO, then diluted in 384-well plates using DMSO followed by a transfer of the dilutions into in HBSS containing 0.1% bovine serum albumin (BSA), NaHCO₃: 0.375 g/l and 20 mM HEPES. On the day of the assay, 50 μl of staining buffer (HBSS containing 1% FCS, 20 mM HEPES, NaHCO₃: 0.375 g/l, 5 mM probenecid (Sigma) and 3 μM of the fluorescent calcium indicator fluo-4 AM (1 mM stock solution in DMSO, containing 10% pluronic) is added to each well. The 384-well cell-plates are incubated for 50 min at 37° C. in 5% CO₂ followed by equilibration at r.t. for 30-120 min before measurement.

Within the Fluorescent Imaging Plate Reader (FLIPR Tetra, Molecular Devices), antagonists are added to the plate in a volume of 10 μl/well, incubated for 10 min and finally 10 μl/well of agonist is added. Fluorescence is measured for each well at 1 second intervals, and the height of each fluorescence peak is compared to the height of the fluorescence peak induced by 3 nM orexin-A with vehicle in place of antagonist. For each antagonist, the IC₅₀ value (the concentration of compound needed to inhibit 50% of the agonistic response) is determined and may be normalized using the obtained IC₅₀ value of a on-plate reference compound. Optimized conditions were achieved by adjustment of pipetting speed and cell splitting regime. The calculated IC₅₀ values of the compounds may fluctuate depending on the daily cellular assay performance. Fluctuations of this kind are known to those skilled in the art.

Antagonistic activities (IC₅₀ values) of all exemplified compounds are below 10000 nM with respect to the OX₁ and/or the OX₂ receptor. With respect to the OX₁ receptor, IC₅₀ values of 154 exemplified compounds are in the range of 4-9686 nM with an average of 892 nM; An IC₅₀ value of one compound has been measured >10000 nM. With respect to the OX₂ receptor, IC₅₀ values of 154 exemplified compounds are in the range of 1-9659 nM with an average of 1113 nM. The ICₛₒ value of one compound has not been measured. Antagonistic activities of selected compounds are displayed in Table 1.

TABLE 1

| Compound of | OX₁ IC₅₀ | OX₂ IC₅₀ |
|---|---|---|
| Example | (nM) | (nM) |
| 1 | 32*² | 5*² |
| 2 | 42 | 25 |
| 10 | 61 | 89 |
| 22 | 48 | 115 |
| 46 | 11*³ | 6*³ |
| 62 | 3440 | 1800 |
| 76 | 363 | 165 |
| 77 | 295 | 613 |
| 85 | 1812 | 146 |
| 89 | 44 | 47 |
| 115 | 25 | 41 |
| 139 | 796*² | 530*² |
| 141 | 85 | 19 |
| 147 | 47 | 8302 |
| 152 | 63 | 32 |
| 154 | 7250 | 654 |

*²geometric mean of n = 2 values;
*³geometric mean of n = 3 values;

1. A compound of formula (I) 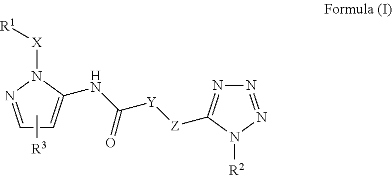
wherein
X represents —CH₂—, —CH₂—CH₂—, —CH₂—CH₂—CH₂—, or a bond;
Y represents —CH₂— which is optionally mono-substituted with (C₁₋₄)alkyl;
Z represents —CH₂—, or —S—;
R¹ represents aryl or heteroaryl, wherein said aryl or heteroaryl independently is unsubstituted, or mono-, di-, or tri-substituted, wherein the substituents are independently selected from the group consisting of (C₁₋₄)alkyl; (C₁₋₄)alkoxy; fluoroalkyl; fluoroalkoxy; halogen; N(CH₃)₂; phenyl and phenyloxy, wherein said phenyl or phenyloxy independently is unsubstituted or mono-, or di-substituted wherein the substituents are independently selected from the group consisting of (C₁₋₄)alkyl, (C₁₋₄)alkoxy, fluoroalkyl, fluoroalkoxy, and halogen; or,
in case X represents —CH₂—, R¹ additionally represents (C₁₋₆)alkyl, or (C₃₋₆)cycloalkyl;
R² represents phenyl which is unsubstituted; or mono-, di-, or tri-substituted, wherein the substituents are independently selected from the group consisting of (C₁₋₄)alkyl, (C₁₋₄)alkoxy, halogen, fluoroalkyl, and fluoroalkoxy; or
R² represents a naphthyl group or a biphenyl group which groups independently are unsubstituted, or mono-, or di-substituted, wherein the substituents are independently selected from the group consisting of (C₁₋₄)alkyl, (C₁₋₄)alkoxy, fluoroalkyl, fluoroalkoxy, and halogen; and
R³ represents hydrogen or methyl;
or a salt thereof;
with the exception of the following compounds:
N-(2-Benzyl-2H-pyrazol-3-yl)-2-[1-(2,5-dimethyl-phenyl)-1H-tetrazol-5-ylsulfanyl]-acetamide;
2-[1-(2,5-Dimethyl-phenyl)-1H-tetrazol-5-ylsulfanyl]-N-(2-thiophen-2-ylmethyl-2H-pyrazol-3-yl)-acetamide;
N-[2-(3-Chloro-benzyl)-2H-pyrazol-3-yl]-2-(1-phenyl-1H-tetrazol-5-ylsulfanyl)-acetamide;
N-[2-(2,5-Dimethyl-phenyl)-5-methyl-2H-pyrazol-3-yl]-2-(1-phenyl-1H-tetrazol-5-ylsulfanyl)-acetamide;
N-(5-Methyl-2-phenyl-2H-pyrazol-3-yl)-2-(1-m-tolyl-1H-tetrazol-5-ylsulfanyl)-acetamide;
N-[2-(4-Fluoro-phenyl)-5-methyl-2H-pyrazol-3-yl]-2-(1-phenyl-1H-tetrazol-5-ylsulfanyl)-acetamide;
2-[1-(2,5-Dimethyl-phenyl)-1H-tetrazol-5-ylsulfanyl]-N-(5-methyl-2-phenyl-2H-pyrazol-3-yl)-acetamide;
2-[1-(3,5-Dimethyl-phenyl)-1H-tetrazol-5-ylsulfanyl]-N-(5-methyl-2-p-tolyl-2H-pyrazol-3-yl)-acetamide;
2-[1-(2,4-Dimethyl-phenyl)-1H-tetrazol-5-ylsulfanyl]-N-(2,5-dimethyl-2H-pyrazol-3-yl)-acetamide;
N-(5-Methyl-2-p-tolyl-2H-pyrazol-3-yl)-2-[1-(4-trifluoromethoxy-phenyl)-1H-tetrazol-5-ylsulfanyl]-acetamide;
2-[1-(2,5-Dimethyl-phenyl)-1H-tetrazol-5-ylsulfanyl]-N-(5-methyl-2-o-tolyl-2H-pyrazol-3-yl)-acetamide;
2-[1-(2,6-Dimethyl-phenyl)-1H-tetrazol-5-ylsulfanyl]-N-(5-methyl-2-phenyl-2H-pyrazol-3-yl)-acetamide;
2-[1-(3-Fluoro-phenyl)-1H-tetrazol-5-ylsulfanyl]-N-(5-methyl-2-phenyl-2H-pyrazol-3-yl)-acetamide;
2-[1-(3,4-Dimethyl-phenyl)-1H-tetrazol-5-ylsulfanyl]-N-(5-methyl-2-phenyl-2H-pyrazol-3-yl)-acetamide;
N-(5-Methyl-2-phenyl-2H-pyrazol-3-yl)-2-(1-o-tolyl-1H-tetrazol-5-ylsulfanyl)-acetamide;
N-(5-Methyl-2-phenyl-2H-pyrazol-3-yl)-2-(1-p-tolyl-1H-tetrazol-5-ylsulfanyl)-acetamide;
2-[1-(2,3-Dimethyl-phenyl)-1H-tetrazol-5-ylsulfanyl]-N-(5-methyl-2-phenyl-2H-pyrazol-3-yl)-acetamide;
2-[1-(2,4-Dimethyl-phenyl)-1H-tetrazol-5-ylsulfanyl]-N-(5-methyl-2-phenyl-2H-pyrazol-3-yl)-acetamide; and
N-(5-Methyl-2-phenyl-2H-pyrazol-3-yl)-2-(1-phenyl-1H-tetrazol-5-ylsulfanyl)-acetamide.

2. A compound according to claim 1, wherein X represents —CH₂—, —CH₂—CH₂—, or —CH₂—CH₂—CH₂—; or a salt thereof.

3. A compound according to claim 1, wherein Y represents —CH₂—; or a salt thereof.

4. A compound according to claim 1, wherein R¹ represents aryl or heteroaryl, wherein said aryl or heteroaryl independently is unsubstituted, or mono-, di-, or tri-substituted, wherein the substituents are independently selected from the group consisting of (C₁₋₄)alkyl; (C₁₋₄)alkoxy; fluoroalkyl; fluoroalkoxy; halogen; N(CH₃)₂; phenyl and phenyloxy, wherein said phenyl or phenyloxy independently is unsubstituted or mono-, or di-substituted wherein the substituents are independently selected from the group consisting of (C₁₋₄)alkyl, (C₁₋₄)alkoxy, fluoroalkyl, fluoroalkoxy, and halogen;
or a salt thereof.

5. A compound according to claim 1, wherein R² represents phenyl which is unsubstituted; or mono-, di-, or tri-substituted, wherein the substituents are independently selected from the group consisting of (C₁₋₄)alkyl, (C₁₋₄)alkoxy, halogen, fluoroalkyl, and fluoroalkoxy; wherein, in case one substituent is attached to position 4, one further substituent is attached to position 2 of the phenyl ring; or
R² represents a naphthyl group or a biphenyl group which groups independently are unsubstituted, or mono-, or di-substituted, wherein the substituents are independently selected from the group consisting of (C₁₋₄)alkyl, (C₁₋₄)alkoxy, fluoroalkyl, fluoroalkoxy, and halogen;
or a salt thereof.

6. A compound according to claim 1, wherein R² represents phenyl which is mono-, di-, or tri-substituted, wherein the substituents are independently selected from the group consisting of (C₁₋₄)alkyl, (C₁₋₄)alkoxy, halogen, fluoroalkyl, and fluoroalkoxy, wherein one substituent is attached to position 2 of the phenyl ring; or a salt thereof.

7. A compound according to claim 1, wherein R³ represents hydrogen; or a salt thereof.

8. A compound according to claim 1 selected from the group consisting of
2-[1-(2,3-Dimethyl-phenyl)-1H-tetrazol-5-ylsulfanyl]-N-[2-(4-methoxy-benzyl)-2H-pyrazol-3-yl]-acetamide;
2-[1-(2,3-Dimethyl-phenyl)-1H-tetrazol-5-ylsulfanyl]-N-[2-(4-methoxy-3-methyl-benzyl)-2H-pyrazol-3-yl]-acetamide;
2-[1-(2,3-Dimethyl-phenyl)-1H-tetrazol-5-ylsulfanyl]-N-[2-(3-fluoro-4-methoxy-benzyl)-2H-pyrazol-3-yl]-acetamide;
2-[1-(2,3-Dimethyl-phenyl)-1H-tetrazol-5-ylsulfanyl]-N-[2-(4-isopropyl-benzyl)-2H-pyrazol-3-yl]-acetamide;
2-[1-(2,3-Dimethyl-phenyl)-1H-tetrazol-5-ylsulfanyl]-N-[2-(4-methyl-benzyl)-2H-pyrazol-3-yl]-acetamide;
N-[2-(3,4-Dimethoxy-benzyl)-2H-pyrazol-3-yl]-2-[1-(2,3-dimethyl-phenyl)-1H-tetrazol-5-ylsulfanyl]-acetamide;
2-[1-(2,3-Dimethyl-phenyl)-1H-tetrazol-5-ylsulfanyl]-N-[2-(4-trifluoromethoxy-benzyl)-2H-pyrazol-3-yl]-acetamide;
2-[1-(2,3-Dimethyl-phenyl)-1H-tetrazol-5-ylsulfanyl]-N-[2-(4-trifluoromethyl-benzyl)-2H-pyrazol-3-yl]-acetamide;
2-[1-(2,3-Dimethyl-phenyl)-1H-tetrazol-5-ylsulfanyl]-N-[2-(4-ethoxy-benzyl)-2H-pyrazol-3-yl]-acetamide;
2-[1-(2,3-Dimethyl-phenyl)-1H-tetrazol-5-ylsulfanyl]-N-[2-(2-methyl-benzyl)-2H-pyrazol-3-yl]-acetamide;
N-[2-(3-Chloro-4-methyl-benzyl)-2H-pyrazol-3-yl]-2-[1-(2,3-dimethyl-phenyl)-1H-tetrazol-5-ylsulfanyl]-acetamide;
N-[2-(4-tert-Butyl-benzyl)-2H-pyrazol-3-yl]-2-[1-(2,3-dimethyl-phenyl)-1H-tetrazol-5-ylsulfanyl]-acetamide;
2-[1-(2,3-Dimethyl-phenyl)-1H-tetrazol-5-ylsulfanyl]-N-[2-(6-methoxy-pyridin-3-ylmethyl)-2H-pyrazol-3-yl]-acetamide;
2-[1-(2,3-Dimethyl-phenyl)-1H-tetrazol-5-ylsulfanyl]-N-(2-pyridin-4-ylmethyl-2H-pyrazol-3-yl)-acetamide;
N-[2-(4-Dimethylamino-benzyl)-2H-pyrazol-3-yl]-2-[1-(2,3-dimethyl-phenyl)-1H-tetrazol-5-ylsulfanyl]-acetamide;
2-[1-(2,3-Dimethyl-phenyl)-1H-tetrazol-5-ylsulfanyl]-N-(2-thiophen-3-ylmethyl-2H-pyrazol-3-yl)-acetamide;
2-[1-(2,3-Dimethyl-phenyl)-1H-tetrazol-5-ylsulfanyl]-N-(2-thiophen-2-ylmethyl-2H-pyrazol-3-yl)-acetamide;
N-[2-(4-Methoxy-benzyl)-2H-pyrazol-3-yl]-2-(1-o-tolyl-1H-tetrazol-5-ylsulfanyl)-acetamide;
2-[1-(2,4-Dimethyl-phenyl)-1H-tetrazol-5-ylsulfanyl]-N-[2-(4-methoxy-benzyl)-2H-pyrazol-3-yl]-acetamide;
N-[2-(4-Methoxy-benzyl)-2H-pyrazol-3-yl]-2-[1-(2-methoxy-phenyl)-1H-tetrazol-5-ylsulfanyl]-acetamide;
N-[2-(4-Methoxy-benzyl)-2H-pyrazol-3-yl]-2-[1-(3-methoxy-phenyl)-1H-tetrazol-5-ylsulfanyl]-acetamide;
N-[2-(4-Methoxy-benzyl)-2H-pyrazol-3-yl]-2-(1-m-tolyl-1H-tetrazol-5-ylsulfanyl)-acetamide;
2-[1-(2,5-Dimethyl-phenyl)-1H-tetrazol-5-ylsulfanyl]-N-[2-(4-ethoxy-benzyl)-2H-pyrazol-3-yl]-acetamide;
2-[1-(2,5-Dimethyl-phenyl)-1H-tetrazol-5-ylsulfanyl]-N-[2-(4-methoxy-benzyl)-2H-pyrazol-3-yl]-acetamide;
2-[1-(2,6-Dimethyl-phenyl)-1H-tetrazol-5-ylsulfanyl]-N-[2-(4-methoxy-benzyl)-2H-pyrazol-3-yl]-acetamide;
2-[1-(2-Ethyl-6-methyl-phenyl)-1H-tetrazol-5-ylsulfanyl]-N-[2-(4-methoxy-benzyl)-2H-pyrazol-3-yl]-acetamide; and
2-[1-(2,5-Dimethyl-phenyl)-1H-tetrazol-5-ylsulfanyl]-N-[2-(3-fluoro-4-methoxy-benzyl)-2H-pyrazol-3-yl]-acetamide;
or a salt of such a compound.

9. A compound according to claim 1 selected from the group consisting of
2-[1-(2,3-Dimethyl-phenyl)-1H-tetrazol-5-ylsulfanyl]-N-[2-(4-isopropoxy-benzyl)-2H-pyrazol-3-yl]-acetamide;
2-[1-(2,3-Dimethyl-phenyl)-1H-tetrazol-5-ylsulfanyl]-N-[2-(4-propoxy-benzyl)-2H-pyrazol-3-yl]-acetamide;
N-[2-(4-Chloro-benzyl)-2H-pyrazol-3-yl]-2-[1-(2,3-dimethyl-phenyl)-1H-tetrazol-5-ylsulfanyl]-acetamide;
N-[2-(4-Methoxy-benzyl)-2H-pyrazol-3-yl]-2-[1-(2-methoxy-5-methyl-phenyl)-1H-tetrazol-5-ylsulfanyl]-acetamide;
2-[1-(2-Fluoro-phenyl)-1H-tetrazol-5-ylsulfanyl]-N-[2-(4-methoxy-benzyl)-2H-pyrazol-3-yl]-acetamide;
2-[1-(2,5-Dimethyl-phenyl)-1H-tetrazol-5-ylsulfanyl]-N-[2-(4-isopropyl-benzyl)-2H-pyrazol-3-yl]-acetamide;
2-[1-(2,5-Dimethyl-phenyl)-1H-tetrazol-5-ylsulfanyl]-N-[2-(4-methyl-benzyl)-2H-pyrazol-3-yl]-acetamide; and
2-[1-(2,5-Dimethyl-phenyl)-1H-tetrazol-5-ylsulfanyl]-N-[2-(4-methoxy-3-methyl-benzyl)-2H-pyrazol-3-yl]-acetamide;
or a salt of such a compound.

10. A compound according to claim 1 selected from the group consisting of
2-[1-(2-Chloro-phenyl)-1H-tetrazol-5-ylsulfanyl]-N-[2-(4-methoxy-benzyl)-2H-pyrazol-3-yl]-acetamide;
2-[1-(2,5-Dichloro-phenyl)-1H-tetrazol-5-ylsulfanyl]-N-[2-(4-methoxy-benzyl)-2H-pyrazol-3-yl]-acetamide;
2-[1-(3,5-Dimethyl-phenyl)-1H-tetrazol-5-ylsulfanyl]-N-[2-(4-methoxy-benzyl)-2H-pyrazol-3-yl]-acetamide;
2-[1-(3-Chloro-phenyl)-1H-tetrazol-5-ylsulfanyl]-N-[2-(4-methoxy-benzyl)-2H-pyrazol-3-yl]-acetamide;
2-[1-(2,3-Dimethyl-phenyl)-1H-tetrazol-5-ylsulfanyl]-N-(2-phenethyl-2H-pyrazol-3-yl)-acetamide;
2-[1-(2,3-Dimethyl-phenyl)-1H-tetrazol-5-ylsulfanyl]-N-[2-(5-methoxy-pyridin-3-ylmethyl)-2H-pyrazol-3-yl]-acetamide;
2-[1-(2,3-Dimethyl-phenyl)-1H-tetrazol-5-ylsulfanyl]-N-[2-(3-phenyl-propyl)-2H-pyrazol-3-yl]-acetamide;
2-[1-(2,3-Dimethyl-phenyl)-1H-tetrazol-5-ylsulfanyl]-N-[2-(4-phenoxy-benzyl)-2H-pyrazol-3-yl]-acetamide;
N-[2-(4-Benzyloxy-benzyl)-2H-pyrazol-3-yl]-2-[1-(2,3-dimethyl-phenyl)-1H-tetrazol-5-ylsulfanyl]-acetamide;
N-{2-[3-(2,5-Difluoro-4-methoxy-phenyl)-propyl]-2H-pyrazol-3-yl}-2-[1-(2,3-dimethyl-phenyl)-1H-tetrazol-5-ylsulfanyl]-acetamide;
N-[2-(2,3-Dihydro-benzofuran-5-ylmethyl)-2H-pyrazol-3-yl]-2-[1-(2,6-dimethyl-phenyl)-1H-tetrazol-5-ylsulfanyl]-acetamide;
2-[1-(2,5-Dimethyl-phenyl)-1H-tetrazol-5-ylsulfanyl]-N-(2-phenethyl-2H-pyrazol-3-yl)-acetamide;
2-[1-(2,6-Dimethyl-phenyl)-1H-tetrazol-5-ylsulfanyl]-N-[2-(3-phenyl-propyl)-2H-pyrazol-3-yl]-acetamide;
2-[1-(2,6-Difluoro-phenyl)-1H-tetrazol-5-ylsulfanyl]-N-[2-(4-methoxy-benzyl)-2H-pyrazol-3-yl]-acetamide;
2-[1-(2,6-Diethyl-phenyl)-1H-tetrazol-5-ylsulfanyl]-N-[2-(4-methoxy-benzyl)-2H-pyrazol-3-yl]-acetamide;
2-[1-(2,6-Diisopropyl-phenyl)-1H-tetrazol-5-ylsulfanyl]-N-[2-(4-methoxy-benzyl)-2H-pyrazol-3-yl]-acetamide;
2-[1-(2,6-Dichloro-phenyl)-1H-tetrazol-5-ylsulfanyl]-N-[2-(4-methoxy-benzyl)-2H-pyrazol-3-yl]-acetamide;
2-[1-(2-Chloro-6-methyl-phenyl)-1H-tetrazol-5-ylsulfanyl]-N-[2-(4-methoxy-benzyl)-2H-pyrazol-3-yl]-acetamide;
N-[2-(4-Methoxy-benzyl)-2H-pyrazol-3-yl]-2-[1-(2,4,6-trimethyl-phenyl)-1H-tetrazol-5-ylsulfanyl]-acetamide;
2-[1-(2-Fluoro-5-methyl-phenyl)-1H-tetrazol-5-ylsulfanyl]-N-[2-(4-methoxy-benzyl)-2H-pyrazol-3-yl]-acetamide;
2-[1-(3-Fluoro-2-methyl-phenyl)-1H-tetrazol-5-ylsulfanyl]-N-[2-(4-methoxy-benzyl)-2H-pyrazol-3-yl]-acetamide;
N-[2-(4-Methoxy-benzyl)-2H-pyrazol-3-yl]-2-[1-(2,3,5-trifluoro-phenyl)-1H-tetrazol-5-ylsulfanyl]-acetamide;
2-[1-(5-Fluoro-2-methyl-phenyl)-1H-tetrazol-5-ylsulfanyl]-N-[2-(4-methoxy-benzyl)-2H-pyrazol-3-yl]-acetamide;
N-[2-(4-Methoxy-benzyl)-2H-pyrazol-3-yl]-2-[1-(2-trifluoromethoxy-phenyl)-1H-tetrazol-5-ylsulfanyl]-acetamide;
2-[1-(2,3-Dichloro-phenyl)-1H-tetrazol-5-ylsulfanyl]-N-[2-(4-methoxy-benzyl)-2H-pyrazol-3-yl]-acetamide;
3-[1-(2,3-Dimethyl-phenyl)-1H-tetrazol-5-yl]-N-[2-(4-methoxy-benzyl)-2H-pyrazol-3-yl]-propionamide;
3-[1-(2,5-Dimethyl-phenyl)-1H-tetrazol-5-yl]-N-[2-(4-methoxy-benzyl)-2H-pyrazol-3-yl]-propionamide;
3-[1-(2,6-Dimethyl-phenyl)-1H-tetrazol-5-yl]-N-[2-(4-methoxy-benzyl)-2H-pyrazol-3-yl]-propionamide;
N-[2-(4-Methoxy-benzyl)-2H-pyrazol-3-yl]-3-[1-(2,4,6-trimethyl-phenyl)-1H-tetrazol-5-yl]-propionamide;
N-[2-(4-Methoxy-benzyl)-2H-pyrazol-3-yl]-3-(1-naphthalen-1-yl-1H-tetrazol-5-yl)-propionamide;
3-[1-(2,6-Diethyl-phenyl)-1H-tetrazol-5-yl]-N-[2-(4-methoxy-benzyl)-2H-pyrazol-3-yl]-propionamide;
3-[1-(2,3-Dimethyl-phenyl)-1H-tetrazol-5-yl]-N-[2-(4-isopropyl-benzyl)-2H-pyrazol-3-yl]-propionamide;
3-[1-(2,5-Dimethyl-phenyl)-1H-tetrazol-5-yl]-N-[2-(4-isopropyl-benzyl)-2H-pyrazol-3-yl]-propionamide;
3-[1-(2,6-Dimethyl-phenyl)-1H-tetrazol-5-yl]-N-[2-(4-isopropyl-benzyl)-2H-pyrazol-3-yl]-propionamide;
3-[1-(2,6-Diethyl-phenyl)-1H-tetrazol-5-yl]-N-[2-(4-methoxy-benzyl)-5-methyl-2H-pyrazol-3-yl]-propionamide;
2-[1-(2,3-Dimethyl-phenyl)-1H-tetrazol-5-ylsulfanyl]-N-[2-(4-methoxy-benzyl)-5-methyl-2H-pyrazol-3-yl]-acetamide;
3-[1-(2,6-Diethyl-phenyl)-1H-tetrazol-5-yl]-N-[2-(4-methoxy-benzyl)-4-methyl-2H-pyrazol-3-yl]-propionamide;
2-[1-(2,5-Dimethyl-phenyl)-1H-tetrazol-5-ylsulfanyl]-N-[2-(4-methoxy-phenyl)-2H-pyrazol-3-yl]-acetamide;
2-[1-(2,3-Dimethyl-phenyl)-1H-tetrazol-5-ylsulfanyl]-N-[2-(4-isopropyl-phenyl)-2H-pyrazol-3-yl]-acetamide;
2-[1-(2,3-Dimethyl-phenyl)-1H-tetrazol-5-ylsulfanyl]-N-[2-(4-fluoro-phenyl)-2H-pyrazol-3-yl]-acetamide;
2-[1-(2,3-Dimethyl-phenyl)-1H-tetrazol-5-ylsulfanyl]-N-[2-(3-methoxy-phenyl)-2H-pyrazol-3-yl]-acetamide;
2-[1-(2,3-Dimethyl-phenyl)-1H-tetrazol-5-ylsulfanyl]-N-[2-(4-phenoxy-phenyl)-2H-pyrazol-3-yl]-acetamide;
2-[1-(2,3-Dimethyl-phenyl)-1H-tetrazol-5-ylsulfanyl]-N-(2-p-tolyl-2H-pyrazol-3-yl)-acetamide;
2-[1-(2,3-Dimethyl-phenyl)-1H-tetrazol-5-ylsulfanyl]-N-[2-(3-fluoro-4-methoxy-phenyl)-2H-pyrazol-3-yl]-acetamide;
2-[1-(2,3-Dimethyl-phenyl)-1H-tetrazol-5-ylsulfanyl]-N-[2-(6-methoxy-pyridin-3-yl)-2H-pyrazol-3-yl]-acetamide;
N-[2-(4-Methoxy-phenyl)-2H-pyrazol-3-yl]-2-[1-(2,4,6-trimethyl-phenyl)-1H-tetrazol-5-ylsulfanyl]-acetamide;
N-[2-(4-Methoxy-phenyl)-2H-pyrazol-3-yl]-2-(1-naphthalen-1-yl-1H-tetrazol-5-ylsulfanyl)-acetamide;
2-[1-(2,6-Dichloro-phenyl)-1H-tetrazol-5-ylsulfanyl]-N-[2-(4-methoxy-phenyl)-2H-pyrazol-3-yl]-acetamide; and
2-[1-(2,6-Dimethyl-phenyl)-1H-tetrazol-5-ylsulfanyl]-N-[2-(4-methoxy-phenyl)-2H-pyrazol-3-yl]-acetamide;
or a salt of such a compound.

11. A pharmaceutical composition containing, a compound according to claim 1, or a pharmaceutically acceptable salt thereof, and at least one therapeutically inert excipient.

12. (canceled)

13. A method for the prophylaxis or treatment of a disease selected from the group consisting of all types of sleep disorders, of stress-related syndromes, of addictions, of cognitive dysfunctions in the healthy population and in psychiatric and neurologic disorders, of eating or drinking disorders comprising administering a compound according to claim 1 to a patent at risk for said disease, or in need of treatment for said disease.

14. (canceled)
